SEQUENCE LISTING (3 sequences)

SEQ ID NO: 1 - DNA, 23 bp, Artificial Sequence
  Synthetic
aagucggcggcucugagguactt

SEQ ID NO: 2 - DNA, 23 bp, Artificial Sequence
  Synthetic
aagucagcugcucucagguactt

SEQ ID NO: 3 - DNA, 21 bp, Artificial Sequence
  Synthetic
gcgcgcuuuguaggauucgtt

(19) United States
(12) Patent Application Publication
(10) Pub. No.: US 2011/0054236 A1
(43) Pub. Date: Mar. 3, 2011

(54) COMPOSITIONS AND METHODS FOR TARGETING TUMORS

(75) Inventors: Victor C. Yang, Ann Arbor, MI (US); Beata Chertok, Somerville, MA (US); Allan E. David, Ypsilanti, MI (US)

(73) Assignee: THE REGENTS OF THE UNIVERSITY OF MICHIGAN, Ann Arbor, MI (US)

(21) Appl. No.: 12/857,986

(22) Filed: Aug. 17, 2010

Related U.S. Application Data
(60) Provisional application No. 61/236,686, filed on Aug. 25, 2009.

(51) Int. Cl. A61N 2/00; B82Y 5/00

(52) U.S. Cl. 600/9; 977/773

(57) ABSTRACT

The present invention relates to functionalized magnetic nanoparticles. In particular, the present invention provides functionalized magnetic nanoparticles for research and clinical (e.g., targeted treatment) applications.

### CROSS REFERENCE TO RELATED APPLICATIONS

This Application claims priority to U.S. Provisional Patent Application Ser. No. 61/236,686, filed Aug. 25, 2009, hereby incorporated by reference in its entirety.

### FIELD OF THE INVENTION

The present invention relates to functionalized magnetic nanoparticles. In particular, the present invention provides functionalized magnetic nanoparticles for research and clinical (e.g., targeted treatment) applications.

### BACKGROUND OF THE INVENTION

In the United States alone, new brain tumors develop in nearly 2,000 children and 35,000 adults each year. Most brain tumors are primary, meaning that they rarely spread beyond the brain, as opposed to metastasic. Brain tumors can be further divided into benign tumors which grow slowly and do not spread and malignant tumors that spread and invade surrounding tissues aggressively. Among them, 15-35% attribute to glioblastoma multiforme (GBM)—the most aggressive primary brain tumor that has defied all current therapeutic modalities. There is also strong evidence that the incidence of primary brain tumors among the elderly is increasing rapidly (Greig et al., J. Natl. Cancer Inst. 1990, 82:1621-1624). More adults die each year of primary brain tumors than of Hodgkin's disease or multiple sclerosis, making it the third leading cause of death from cancers (Black, New Engl. J. Med. 1991, 324:1471-1476). Patients diagnosed with malignant gliomas have an average life expectancy of 36-48 weeks, and for the last several decades the survival rate has remained similar without dramatic improvement (Black, New Engl. J. Med. 1991, 324:1471-1476).

Treatment of brain tumors faces a unique challenge compared to other types of cancers, due to the fact that not only are they developed within bone-covered structures, thereby having restricted space to expand, but they are also embedded deeply within an organ carrying a multitude of vital functions. Therefore, even a benign tumor can be life-threatening if it is in an area of the brain that controls critical body functions such as breathing or blood circulation. Treatment normally begins with surgical resection and then follows with radiation or chemotherapy. Surgery faces the risk of removing surrounding tissues that may carry vital brain functions, while radiation and chemotherapy can both harm normal tissues that are near or along the treatment path. Indeed, if the tumor is in regions of cerebral hemispheres that control speech, vision, movement or cognition, surgery usually is not recommended. In addition, the use of radiation on children under the age of three is often prohibited because this is a critical time period of brain development. Chemotherapy, on the other hand, has been offering very limited applications, primarily attributed to the palliative response and limited duration of effects due to lack of targeting and selectivity of the drugs.

New methods of targeting and treating brain tumors are needed.

### SUMMARY OF THE INVENTION

The present invention relates to functionalized magnetic nanoparticles. In particular, the present invention provides functionalized magnetic nanoparticles for research and clinical (e.g., targeted treatment) applications.

For example, in some embodiments, the present invention provides a system for targeting brain tumors, comprising: magnetic iron oxide nanoparticles (MIONs) coated with a coating molecule, wherein the coating molecule is non-covalently associated with a brain targeting molecule comprising anti-tumor agent (e.g., a small molecule agent, macromolecular agent, or an siRNA) linked to a cell-penetrating peptide; and an external magnetic field configured to orient MIONs at the site of the brain tumor. In some embodiments, the cell-penetrating peptide comprises a protein transduction domain peptide. In some embodiments, the protein transduction domain peptide is a peptide such as TAT, low molecular weight protamine, or arginine-rich peptides. In some embodiments, the brain targeting molecule comprises polyethyleneime polymer. In some embodiments, the protein transduction domain is low molecular weight protamine. In some embodiments, the coating molecule is a sulfated glycosaminoglycan (e.g., including but not limited to heparin, heparin sulfate, dextran sulfate or a chondroitin sulfated hyaluronic acid). In some embodiments, the cell-penetrating peptide is low molecular weight protamine. In some embodiments, the system further comprises an agent (e.g., protamine) that disrupts the association between the MIONs and the brain targeting molecule. In some embodiments, the system further comprises a permanent magnet mounted to a tapered pole of a dipole electromagnet to divert the magnetic flux lines emanating from the electromagnet poles to generate a local maximum of the magnetic field on the exposed pole magnetic flux density.

In further embodiments, the present invention provides a method of targeting brain tumors, comprising: administering magnetic iron oxide nanoparticles (MIONs) coated with a coating molecule, wherein the coated molecule is non-covalently associated with an brain targeting molecule comprising anti-tumor agent linked to a cell-penetrating peptide to a subject diagnosed with a brain tumor; and orienting the MIONs at the site of the brain tumor with an external magnetic field. In some embodiments, the coating molecule is a sulfated glycosaminoglycan (e.g., including but not limited to heparin, heparin sulfate, dextran sulfate or a chondroitin sulfated hyaluronic acid). In some embodiments, the method further comprises the step of administering an agent that disrupts the association between the MION and the brain targeting molecule. In some embodiments, the administering is intra-arterial administration, intravenous administration or intranasal administration. In some embodiments, the intra-arterial administration comprises inserting a capillary tube into the artery under conditions such that blood flow through the artery is not impeded. In some embodiments, the method further comprises the step of utilizing a permanent magnet mounted to a tapered pole of a dipole electromagnet to divert the magnetic flux lines emanating from the electromagnet poles to generate a local maximum of the magnetic field on the exposed pole magnetic flux density. In some embodiments, the method prevents the formation of vascular embolisms at the site of intra-arterial administration.

Methods, systems, and compositions of the present invention are not limited by the agent (e.g., therapeutic agent, imaging agent) used. In some embodiments, agents are macromolecular in nature. Macromolecular agents are not limited by size or molecular weight of the macromolecule. Molecular weight of macromolecular agents may be less than 5 kDa, 5-50 kDa, 50-100 kDa, 100-200 kDa, 200-300 kDa, 300-400 kDa, 400-500 kDa, 500-750 kDa, greater than 750 kDa. In some embodiments, systems, compositions, or methods of the present invention comprise agent types including but not limited to toxins, nucleic acids (e.g., siRNA, antisense RNA, or other forms of RNAi agents), proteases, enzymes, structural proteins, antibodies, or nonproteinaceous agents (e.g., lipids, carbohydrates, small molecules, and the like). In some embodiments, macromolecule agents comprise multiple subunits (e.g., 2, 3, 4, 5, 6 or more subunits). In some embodiments, multimeric agents comprise subunits of the same type (homomultimer). In some embodiments, multimer agents comprise subunits of more than one type (heteromultimers).

Methods, systems, and compositions of the present invention are not limited by nanoparticle size or shape. In preferred embodiments, nanoparticles are iron oxide nanoparticles. Size of the nanoparticle may be less than 0.5 nm, 0.5-1 nm, 1-5 nm, 5-10 nm, 10-25 nm, 25-50 nm, 50-100 nm, 100-200 nm, 200 nm or more.

### DESCRIPTION OF THE FIGURES

FIG. 1 shows a schematic illustration of an exemplary brain drug delivery system.

FIG. 2 shows MRI scans showing MION accumulation in 9L gliosarcoma [A] with; [B]without magnetic targeting.

FIG. 3: A plot showing linear correlation between the MRI-derived dR2 parameter and ESR-determined MION concentration in excised tissue samples.

FIG. 4 shows MION concentration in excised tumor and contra-lateral brain tissues quantified by ESR. Data expressed as Mean+SE. n=5.

FIG. 5 shows models of: [A] the original; [B] modified magnetic configurations with [C] and [D] of their respective topographic maps generated by the described magnetic setups. [E] & [F] are typical axial MRI scans of the rat head acquired after magnetic targeting with: [E] the original and [F] modified magnetic setups.

FIG. 6 shows [A] MRI head-scans of 9L-glioma bearing rats of: Left: intravenous (I.V.) and Right: carotid artery magnetic targeting; and [B] MION accumulation in tumor and contralateral brain tissues was determined by ESR.

FIG. 7 shows histochemical analysis of cryosections obtained from [T] tumor (Left); [IB] ipsilateral brain (Middle); and [CB] contralatera brain (Right) regions; of PEI-β-Gal/Hep-MION-administered, magnetically-targeted rats (scale bar: 100 μm). The sections were stained with X-Gal staining to visualize localization of β-Gal. The inset was under a higher magnification (scale bar: 25 μm) and capillaries were separately stained with Burstone's staining.

FIG. 8 shows excised tumors from treated mice. From top to the bottom represent tumors excised from mice treated with: Row#1: PBS solution; Row# gelonin; Row#3: TAT-Gel; Row#4: TAT-Gel+heparin; and Row #5: TAT-Gel+.

FIG. 9 shows xenogen IVIS Fluorescence Imaging of exercised brain and olfactory bulb following intra-nasal administration of PBS solution (Control; Lend FITC-protamine (Right) to mice.

FIG. 10 shows photos of reaction mixtures of ODN+LMWP (Left); ODN+SPDP-activated LMWP (Middle); and ODN+LMWP-PEG (Right).

FIG. 11 shows cellular uptake of FITC-labeled AS-ODN (Top); and LMWP-PEG-AS-ODN (Bottom).

FIG. 12 shows magnetophoretic mobility profiles of fluidMAG-D (Top); fluidMAG-CMX (Middle); and CMD-coated100 (Bottom).

FIG. 13 shows analysis of surface amine content of (1) Gara, (2) purified Gara/PEI mixture, and (3) GPEI nanoparticles using ninhydrin colorimetric assay—absorbance of the Ruhemann's purple chromaphore at 570 nm is proportional to the amine content.

FIG. 14 shows characterization of GPEI nanoparticles as compared to the starting material Gara. A, ζ potential at pH 5.5 measured by electrophoretic light scattering. B, particle size distribution determined by dynamic light scattering. C, induced magnetization measured with MPMS-XL SQUID magnetometer.

FIG. 15 shows interaction of G100 and GPEI nanoparticles with 9L-glioma cells. A, optical micrographs of 9L glioma cells incubated with GPEI and G100. Scale bar=20 μm. (Inset: digital magnification (2×) of representative cells demonstrates increased uptake of GPEI compared to G100). B, uptake of GPE1 and G100 nanoparticles by 9L glioma cells quantified by EPR spectroscopy. C, cytotoxicity assessment of GPE1 and G100 nanoparticles to 9L glioma cells using MTT cell viability assay.

FIG. 16 shows representative subsets of axial MRI head-scans of 9L glioma-bearing rats before (baseline) intravenous administration of (A) G100 or (B) GPEI and after magnetic targeting (post-targ).

FIG. 17 shows (A) nanoparticle accumulation in tumor and contra-lateral brain tissues in magnetically targeted rats after intra-carotid administration of G100 or GPEI at a dose of 12 mg Fe/kg; (B) Target selectivity of GPE1 and G100 nanoparticles for tumor versus contra-lateral brain tissue.

FIG. 18 shows a schematic illustration of the design for measurements of the magnetophoretic mobility.

FIG. 19 shows a TEM image of MION for the generation of nMION and pMION.

FIG. 20 shows FTIR data for nMION and pMION.

FIG. 21 shows a schematic illustration of the synthesis of “core-shell” nanoparticles with dual nature of superior magnetophoretic mobility and superparamagnetism.

FIG. 22 shows Changes on (a) zeta potentials; and (b) sizes of the assembled MION cores as well as of the CMD-coated core-shell nanoparticles. In (a), MION cores were positively charged when an excess amount of pMION was used (black triangles). After coating these MION cores with CMD, however, zeta potentials immediately turned into negative values (▪). In (b), both the sizes of the magnetic cores (black triangles) and of the CMD-coated nanoparticles (▪) increased by increasing the ratio of nMION over pMION.

FIG. 23 shows STEM-BF images of (a) CMD-coated100 and (b) fluidMAG-CMX. Changes of carbon (top curve) and iron (lower curve) contents were determined via elemental analysis by EDS linescan along the black line path. The x-axis corresponded to the linescan position whereas y-axis represented the relative abundance of carbon and iron elements.

FIG. 24 shows thermogravimetric analysis (TGA) results of magnetic nanoparticles including the self-assembled nMION/pMION core, CMD-coated100, fluidMAG-CMX, and fluidMAG-D.

FIG. 25 shows superparamagnetic properties measured by SQUID. Both MION and CMD-coated100 particles displayed superparamagnetic behavior in that magnetization and demagnetization curves completely overlapped. In addition, both particles also demonstrated almost identical magnetic susceptibility and saturation magnetization, suggesting that the applied synthesis strategy did not alter the magnetic properties.

FIG. 26 shows MRI images of the brain tumors in (a) control rat without magnetic targeting; and (b) experimental rat with magnetic targeting for 30 min after tail-veil injection of CMD-coated100.

FIG. 27 shows stability of magnetic nanoparticles in a simulated body fluid containing 50% (v/v) calf serum in pH 7.4 PBS. Absorbance at 370 nm was used to monitor the stability of these test nanoparticles.

FIG. 28 shows surface masking of cationic magnetic nanoparticles with LMWH-PEG conjugates.

FIG. 29 shows chromatograms obtained during purification of LMWH-PEG (c) as compared to free PEG (a, control) and free LMWH (b, control) on (A) High-Q anion-exchange column and (B) C8 reverse phase column.

FIG. 30 shows complexation of GPEI with LMWH-PEG conjugates. (A) Inverse linear dependence (R₂ ¼ 0.98) of nanoparticle z-potential (pH ¼ 5.5) on the amount of added LMWP-PEG conjugate indicates charge masking of GPEI cationic surface by ionic complexation. (B) Dependence of the residual LMWH concentrations in the supernatant on the amount of added conjugate indicates saturation of the GPEI surface with the LMWH-PEG conjugate and confirms complexation between these two species.

FIG. 31 shows in vitro stability of GPEI complexed with LMWH-PEG conjugates (test) or free LMWH and PEG in reduced serum medium.

FIG. 32 shows plasma concentration-time profiles for GPEI nanoparticles in rats following intravenous administration of GPEI (▪) with and (∘) without LMWH-PEG complexation. Inset: corresponding area under the concentration-time curve (AUC) of GPEI with (hash bar) and without (solid bar) LMWH-PEG.

FIG. 33 shows representative R2 maps (msec⁻¹) of the tumor region before nanoparticle administration (baseline) and after magnetic targeting (post-targ) in rats injected with (A) GPEI/LMWH-PEG, and (B) GPEI.

FIG. 34 shows quantitative EPR analysis of nanoparticle concentration in excised tumor and contra-lateral brain tissue of rats administered with GPEI/LMWH-PEG and GPEI.

FIG. 35 shows X-ray diffraction pattern of GA-MNP lyophilized powder.

FIG. 36 shows a TEM image of GA coated MNP (scale bar, 100 nm).

FIG. 37 shows magnetization of GA-MNP measured by SQUID.

FIG. 38 shows hydrodynamic particle size of GA-MNP in PBS buffer.

FIG. 39 shows physiologically simulated stability of GA-MNP and several commercial MNP products including: heparin-(Heparine); GA-(GAM); DEAE-(DEAE); CMDX-(CMDX); and starch-coated (Starch) MNP in PBS (containing 10% FBS) medium.

FIG. 40 shows a) Fluorescence microscopy of cellular uptake of RhB-GAMNP (scale bar, 10 μm). B) In vitro gradient echo MR images of 9L glioma cells, from top: 1 control cells, 2 cells treated by GA-MNP in DMEM with 10% FBS, and 3 cells treated by GA-MNP in DMEM without serum. c Gradient echo MR images of GA-MNP at iron concentrations of 0, 5, 10, 20, and 50 ppm (from top to bottom)

FIG. 41 shows cellular uptake of GA-MNP and starch-MNP by 9L glioma cells after 2 h of incubation (n=3).

FIG. 42 shows In vivo magnetic targeting of 9L glioma bearing rats. a-c Representative MR images of the targeted animals. d-f Representative MR images of the non-targeted animals. The arrows in a-f showed the position of tumors. g GA-MNP content in excised brain tumors and normal brain tissues of targeted (n=4) and non-targeted rats (n=3) in the ESR studies.

FIG. 43 shows (A) Representative brain MRI images (Gradient Echo (GE) axial scans) of 9L-glioma bearing rats (1) not exposed to iron oxide nanoparticles (blank) and (2) administered with iron oxide nanoparticles under a gradient of magnetic flux density. (B) ICP-OES analysis of the corresponding excised tumor tissues revealing no statistical difference (p) 0.383) in Fe concentration between tumors of (1) the blank and (2) the nanoparticle-administered rats.

FIG. 44 shows a cryo-handling methodology for introduction of tissue samples into ESR tubes. (A,B) Pushing of the sample into the tube using a glass rod under ambient temperature (A) leads to sample loss due to smearing of the tissue along the tube walls (B). (C,D) In contrast, the cryogenically handled sample is pushed to the bottom of the tube without any tissue loss.

FIG. 45 shows representative ESR spectra of animal tissues excised from magnetically targeted animals (1) and animals not exposed to magnetic nanoparticles (2).

FIG. 46 shows a comparison of ICP-OES and ESR methodologies for MNP concentration analysis of (A) liver and (B) spleen obtained from rats which were administered with different concentrations of magnetic nanoparticles within the range of 12-25 mg Fe/kg.

FIG. 47 shows (A) ICP-OES and (B) ESR analysis of high MNP accumulating organs from animals administered with MNP under magnetic targeting (test) and animals not exposed to MNP (blank). Data of ICP-OES analysis.

FIG. 48 shows (A) ICP-OES and (B) ESR analysis of low MNP accumulating organs from animals administered with MNP under magnetic targeting (test) and animals not exposed to MNP (blank).

FIG. 49 shows biodistribution profiles of MNP in magnetically targeted animals obtained with (A) ICP-OES and (B) ESR methodologies.

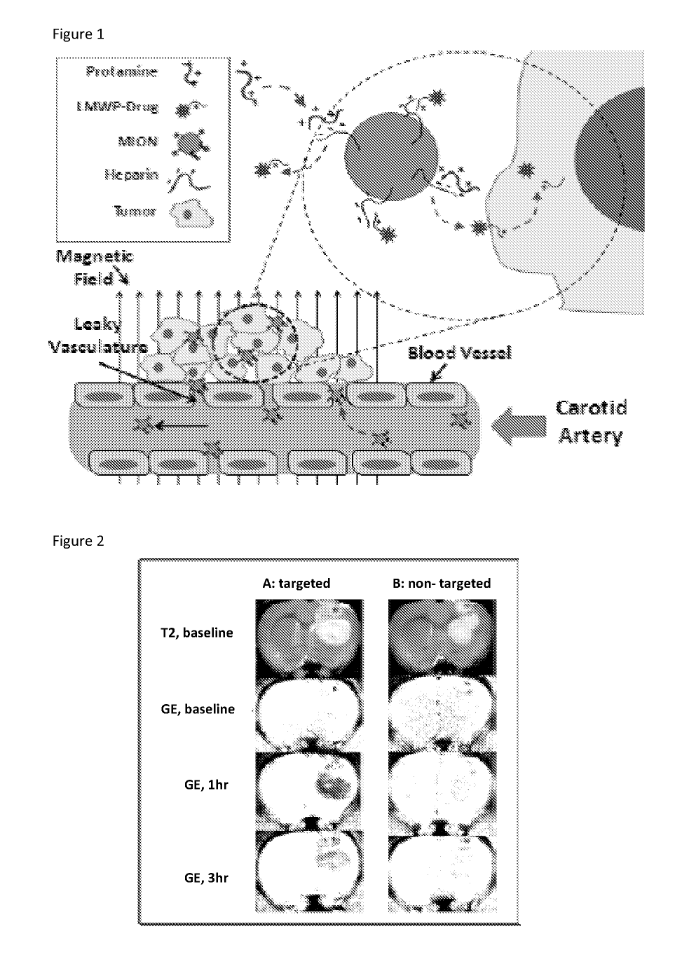

### DEFINITIONS

As used herein, the term “agent” refers to a composition that possesses a biologically relevant activity or property. Biologically relevant activities are activities associated with biological reactions or events or that allow for the detection, monitoring, or characterization of biological reactions or events. Biologically relevant activities include, but are not limited to, therapeutic activities (e.g., the ability to improve biological health or prevent the continued degeneration associated with an undesired biological condition), targeting activities (e.g., the ability to bind or associate with a biological molecule or complex), monitoring activities (e.g., the ability to monitor the progress of a biological event or to monitor changes in a biological composition), imaging activities (e.g., the ability to observe or otherwise detect biological compositions or reactions), and signature identifying activities (e.g., the ability to recognize certain cellular compositions or conditions and produce a detectable response indicative of the presence of the composition or condition). The agents of the present invention are not limited to these particular illustrative examples. Indeed any useful agent may be used including agents that deliver or destroy biological materials, cosmetic agents, and the like.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, RNA (e.g., including but not limited to, mRNA, tRNA and rRNA) or precursor. The polypeptide, RNA, or precursor can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) processed transcript. The mRNA functions during translation to specify the sequence or order of amino acids in a nascent polypeptide.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” “polymorphism,” and “variant” refer to a gene or gene product that displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product. As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain by virtue of the well established genetic code. The DNA sequence thus codes for the amino acid sequence.

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding of the compositions (claimed in the present invention) with its various ligands and/or substrates.

As used herein, the term “purified” or “to purify” refers to the removal of contaminants from a sample. For example, antibodies are purified by removal of contaminating non-immunoglobulin proteins; they are also purified by the removal of immunoglobulin that does not bind the target protein. The removal of non-immunoglobulin proteins and/or the removal of immunoglobulins that do not bind the target protein results in an increase in the percent of target reactive immunoglobulins in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source. The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a Cᵢ₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

### DETAILED DESCRIPTION OF THE INVENTION

The present invention relates to functionalized magnetic nanoparticles. In particular, the present invention provides functionalized magnetic nanoparticles for diagnostic, research and clinical (e.g., targeted treatment) applications.

In some embodiments, nanoparticles are treated with compounds (e.g., citrate, glycine) to produce negatively or positively charged surfaces, respectively. These charged nanoparticles remain under the size threshold essential for retaining superparamagentic behavior without forming tightly bound aggregates, because the strong inter-particle magnetic attraction is offset by the charge-induced repulsive force. Self-assembly of these −/+charged nanoparticles then takes place via electrostatic interaction to yield a loosely agglomerated magnetic core possessing both superparamagnetic behavior and high magnetophoretic mobility.

The compositions and methods of the present invention provide the advantage of allowing for intra-arterial injection. Intra-arterial administration offers the advantage of allowing carriers to bypass renal and hepatic clearance during their first passage through the circulation, and was therefore exploited to further enhance brain tumor magnetic targeting. Prior to the present invention, when mice were injected with high concentrations of MION, significant embolism of the afferent vasculature occurred due to their aggregation. Experiments conducted during the course of development of the present invention resulted in the development of a magnetic targeting strategy that optimized the arterial flow dynamics and magnetic field topography and overrode the vascular embolism obstacle.

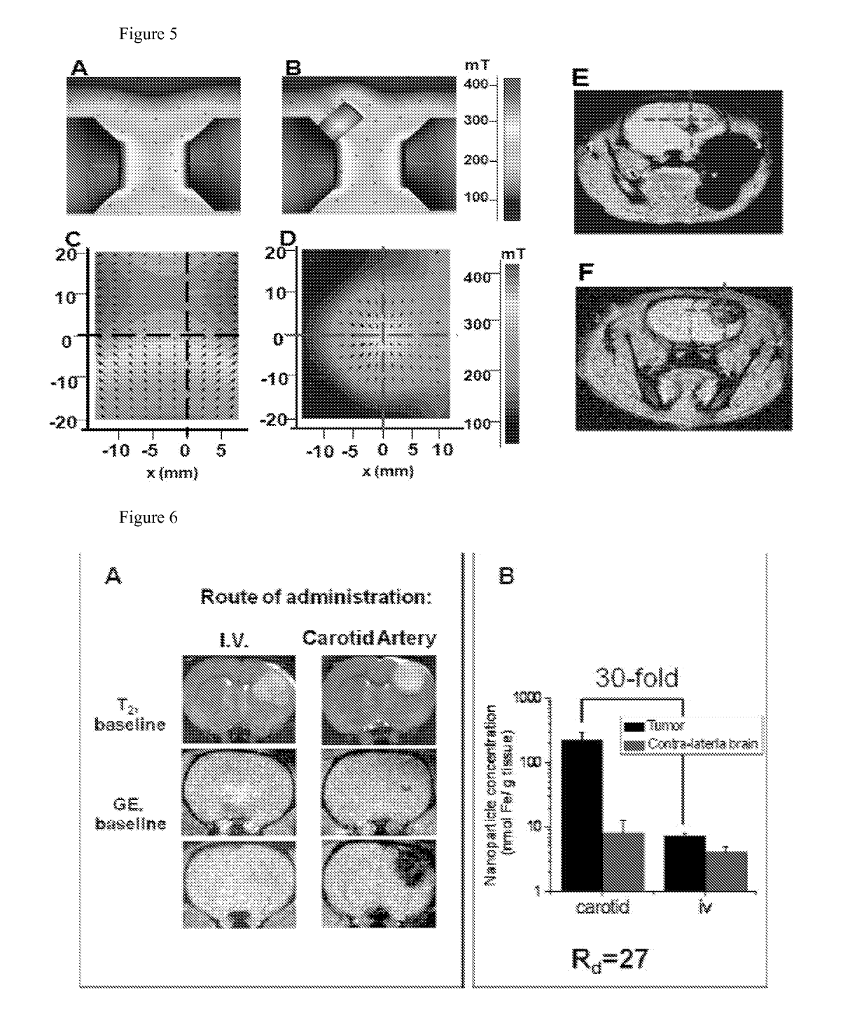

For example, in some embodiments, to maintain carotid flow dynamics, a new catheterization procedure that would not require vessel occlusion was used that comprises inserting a thin silica capillary tubing (OD˜150 μm), which functioned as a needle, through the vascular wall without compromising the integrity of the artery. This catheterization technique did not impede the blood flow through the artery. In other embodiments, an optimized magnet configuration was used to reduce the exposure of MION in the afferent arterial vasculature to magnetic force. In some embodiments, the approach utilizes a small magnet to divert the magnetic flux lines emanating from the electromagnet poles, thus generating a local maximum of the magnetic field on the exposed pole magnetic flux density. Experiments conducted during the course of development of the present invention (See e.g., FIGS. 5E and 5F), demonstrated a 6-fold reduction of the magnetic force at the injection site and alleviated MION aggregation in the afferent vasculature observed previously.

In some embodiments, the present invention provides allows for aligning the tumor lesion with the region of the maximal magnetic flux density by properly positioning the subject with respect to the magnetic setup. In some embodiments, MRI is used to determine the intracerebral localization of the tumor lesion. Using the MRI-derived tumor coordinates allows for positioning the subject in a way that maximizes the exposure of the tumor lesion to the magnetic force, while at the same time minimizing the exposure of the afferent vasculature.

Experiments conducted during the course of development of the present invention demonstrated the targeting efficiency of the systems and methods described herein. Experiments were conducted using both intravenous and intra-arterial administration of polyethyleneimine (PEI)-coated MION (termed PEI-MION). Results (See e.g., FIG. 6A) revealed that while animals administered with PEI-MION via the intravenous route did not show any discerned difference between the post-targeting and the baseline GE scans, animals injected with PEI-MION via carotid artery displayed a pronounced hypointense region in the post-targeting GE MRI scans corresponding to significant MION accumulation in the tumor lesion. In addition, quantitative analysis by ESR of MION accumulation in excised tumor and contra-lateral brain tissues under magnetic targeting (FIG. 6B) displayed that intra-carotid administration of PEI-MION resulted in a 30-fold increase (p=0.002) in tumor capture of PEI-MION compared to that seen with intravenous injection (222.8±66.7 versus 7.3±0.8 nmol Fe/g tissue, respectively). Also observed was a 26-fold difference (p=0.004) in targeting selectivity on brain tumor over the contralateral normal brain (222.8±66.7 versus 8.6±1.0 nmol Fe/g tissue by comparing Columns #1 & #2 from the left of FIG. 6B) following intra-carotid administration.

In addition, quantitative analysis by ESR of MION accumulation in excised tumor and contra-lateral brain tissues under magnetic targeting (FIG. 6B) displayed that intra-carotid administration of PEI-MION resulted in a 30-fold increase (p=0.002) in tumor capture of PEI-MION compared to that seen with intravenous injection (222.8±66.7 versus 7.3±0.8 nmol Fe/g tissue, respectively).

MRI scans showed that, in the absence of magnetic targeting, no clear difference was visually discerned between the post-targeting and the baseline GE brain scans of control animals receiving PEI-β-Gal/Hep-MION but without magnetic targeting. With magnetic targeting, the GE post-targeting scan of experimental animal again displayed a region of pronounced hypointensity, spatially corresponding to the location of the tumor lesion, and thereby validating successful delivery of the (β-Gal-loaded MION to the tumor site.

In experiments conducted during the course of developing some embodiments of the present invention, a size-tunable method to synthesize stable and superamagnetic iron oxide nanoparticles (MION) for both magnetic targeting and MR imaging was developed (Yu et al. (2009) J. Biomed. Materials Res. 92:1468-1475; herein incorporated by reference in its entirety). In other experiments conducted during the course of developing some embodiments of the present invention, the MION thus synthesized displayed artificial peroxidase activity for application in clinical glucose detection (Yu et al. (2009) Biomaterials 30:4716-4722; herein incorporated by reference in its entirety). In other experiments conducted during the course of developing some embodiments of the present invention, the biocompatible gum Arabic polymer was used to coat such magnetic nanoparticles for allowance of conjugation of bioactive agents to these nanoparticles via functional groups on the gum Arabic polymer coating (Lei et al. (2009) AAPS Journal 11:693-699; herein incorporated by reference in its entirety). In other experiments conducted during the course of developing some embodiments of the present invention, via adsorptive surface masking with polyethylene glycol (PEG), substantiated in vivo magnetic brain tumor targeting was observed with the use of PEI-modified MION (Beata Chertok et al. (2009) Biomaterials 30:6780-6787; herein incorporated by reference in its entirety). In other experiments conducted during the course of developing some embodiments of the present invention, electron spin resonance (ESR) and inductively coupled plasma optical emission spectroscopy (ICP-EOS) methods were developed and compared while studying biodistribution of MION in magnetically targeted rats (Beata Chertok et al. (2010) Mol. Pharmaceutics. 7:375-385; herein incorporated by reference in its entirety). In other experiments conducted during the development of some embodiments of the present invention, the transcellular transport phenomenon of self-synthesized heparin-coated MION (Hep-MION) under the influence of an external magnetic field was analyzed (Ah Min et al. (2010) Pharmaceutics 2:119-135; herein incorporated by reference in its entirety). In other experiments conducted during the development of some embodiments of the present invention, to further augment desired properties (e.g., targeting properties, imaging properties), MION with large core-shell structure possessing dual functionalities of enhanced magnetophoretic mobility and superparamagnetism were synthesized (Beata Chertok et al. (2010) Biomaterials 31:3617-3624; herein incorporated by reference in its entirety). In other experiments conducted during the course of developing some embodiments of the present invention, an intra-arterial MION administration method was applied to bypass the first pass organ clearance, facilitating augmented MION accumulation at the brain tumor site (e.g., by nearly 35-fold) (Yu et al. (2010) Biomaterials 31:5842-5848; herein incorporated by reference in its entirety). Via this topography-optimized magnetic tumor targeting strategy, a significant amount of the large (465 kDa) β-galactosidase was delivered selectively into a brain tumor but not to the ipsi-lateral or contra-lateral normal brain regions, e.g., as described herein.

### I. Nanoparticles

Embodiments of the present invention provide magnetic nanoparticles for use in research and clinical applications. In some embodiments, the nanoparticles are magnetic iron oxide nanoparticles (MIONs).

In some embodiments, MIONs comprise a nanomaterial which includes a magnetic nanocomponent coated by a single or multiple layer(s) of non-toxic metal oxide(s), with or without inclusion of quantum dot materials; completed by a bio-inert surface coating with or without addition of bioactive polymers or bio-molecules, depending on the different application purposes.

In some embodiments, the magnetic nanocomponent in the nanomaterials is a material that exhibits diamagnetism, ferrimagnetism, ferromagnetism, paramagnetism, superdiamagnetism, or superparamagnetism nanomaterials properties. In some embodiments, the magnetic nanocomponent introduces changes in T₁ and/or T₂ relaxation time to offer a high-contrast effect for MRI.

In some embodiments, the magnetic nanocomponent in the nanomaterial is a precursor for α-Fe₂O₃, γ-Fe₂O₃ or related nanoalloy oxides with Fe after oxidization or for bcc-Fe (body-centered cubic-Fe) or alloys-based Fe nanocomponents after reducing. The magnetic nanocomponent in the nanomaterials based on iron oxide can be extended to other iron oxide based nanomaterials, including, but not limited to, MFe₂O₄, RFeO₃, and MRFeOₓ (M=Ba, Bi, Co, Cr, Cu, Fe, Mg, Mn, Ni, Ti, Y, Zn) (R=rare earth metal elements) nanomaterials, and iron oxide coated various nanomaterials. In some embodiments, nanomaterials are FeO₂ nanoparticles.

The size of the completed nanomaterials in at least one dimension is preferably within 0.1-1000 nm. The shape of the nanomaterials may be regular (column, cube, cylinder, pillar, pyramid, rod, sphere, tube etc.) or irregular/random. The shape of the nanomaterials is controlled by adjusting the reaction dynamics and aging/ripening time.

Application of magnetic nanoparticles (e.g., MIONs) as the drug carriers for tumor targeting has recently drawn attention. With the aid of an external magnetic field, this approach can deliver drugs site-specifically. MION can penetrate through the capillary endothelium of the tumor (Zimmer et al., Exp. Neurol. 1997; 143:61-69; Moore et al., Radiology 2000; 214:568-574; each herein incorporated by reference in its entirety) via the EPR (Enhanced Permeability and Retention) effect (Kreuter et al., Brain Res. 1995; 674:171-174; herein incorporated by reference in its entirety). With the aid of an externally applied magnetic field, the use of MION allows conversion of conventional passive tumor targeting into an active magnetic targeting and provides magnetic-enhanced retention of MION once they accumulate at the tumor interstitium. It was reported that MIONs were detectable in the brain tumor after intravenous administration (Pulfer et al., J. Neuro-Oncol. 1999; 41:99-105; Reddy et al., Clin. Cancer Res. 2006; 12:6677-86; each herein incorporated by reference in its entirety). Certain magnetic nanoparticles such as MION are strong enhancers of proton spin-spin (T2/T*2) relaxation, and the resulted reduction in signal intensity due to accumulation of MION aids drug therapy.

Magnetic nanoparticles used for biomedical applications generally comprise a composite where particles of a magnetic component are coated with a polymeric shell. The overall size of the composite, referred to as the hydrodynamic diameter, is different from the size of the core of magnetic crystals, which are mainly responsible for the magnetism of the composite. The magnetic core is made e.g. by iron oxide compound comprising of a mixture of magnetite (Fe₃O₄) and maghemite (Fe₂O₃), or by transition metals such as Ni and Co. The polymeric shell often consists of a biocompatible polymer such as dextran or starch. Iron oxide-based nanoparticles, herein termed MION, are non-toxic and possess favorable tolerability profiles. Clinical studies have shown that dextran-coated MIONs are completely biodegradable, biocompatible and without acute or sub-acute effects following use on animals (Harisinghani et al., AJR 1999; 172:1347-1351; Shen et al., Magn. Reson. Med. 1993; 29:599-604; Weissleder et al., AJR 1989; 152:167-173; each herein incorporated by reference in its entirety). Pharmacokinetic and toxicological studies indicated that the dextran coating was enzymatically degraded, and MION was dissolved to the form of iron which then incorporated into hemoglobin of erythrocytes and cleared primarily from liver and spleen (t_{1/2}: 3-4 days) (Harisinghani et al., supra). In general, MIONs greater than 200 nm in diameter are sequestered in the spleen and removed by the RES, and MIONs smaller than 10 nm are cleared by renal filtration (Huynh et al., J. Control Release 2006; 110:236-59; herein incorporated by reference in its entirety). MIONs between 100-200 nm are known to possess a long-circulating plasma half-life for tumor imaging (Enochs et al., J Mag Reson Imaging. 1999; 9:228-32; herein incorporated by reference in its entirety).

In some embodiments, for clinical applications, MIONs are superparamagnetic in order to avoid aggregation prior to and during its application. MIONs exhibit superparamagnetic behavior below certain size threshold. It has been demonstrated that magnetic properties of magnetic nanoparticles become weaker with reduction in size. Saturation magnetization decreases linearly with decreasing crystallite size (Morales et al., J. Magnetism Magn. Mater. 1999; 203:146-148; herein incorporated by reference in its entirety). Whilst small superparamagnetic nanoparticles can still retain function in MRI (Josephson et al. (1983) Magn. Reson. Imaging 6:647-53; herein incorporated by reference in its entirety), they cannot be withdrawn from the fluid carrier (e.g. blood) under clinically adoptable magnetic field gradients (Bean, J. Appl. Phys. 1959; 30:120S-129S; herein incorporated by reference in its entirety). In contrast, the efficiency of magnetic targeting depends primarily on the magnetophoretic mobility, a parameter that can be enhanced by increasing the size of the magnetic core.

In some embodiments, MIONs are coated with a molecule that functions to bind the cell-penetrating peptides therefore inhibiting the cell internalization activity of such peptides. In some embodiments, coating molecules are sulfated glycosaminoglycans (e.g., including but not limited to, heparin, heparin sulfate, dextran sulfate, chondroitin sulfated hyaluronic acids, etc.). In some embodiments, MIONs are coated with a negatively charged molecule (e.g., heparin).

In some embodiments, a therapeutic agent (e.g., tumor targeting agent) is associated with the MIONs. In some embodiments, the therapeutic agent has the opposite charge of the molecule coating the MION (e.g., a positive charge) so that the therapeutic agent can interact with the negatively charged molecule coating the MION via electrostatic interactions.

In some embodiments, the therapeutic agent comprises a drug and a cell penetrating peptide or molecule. In some embodiments, the cell penetrating peptide is low molecular weight protamine, although other cell penetrating peptide may be used.

The present invention is not limited to a particular drug. In some embodiments, the drug is a known chemotherapeutic agent (see below). In some embodiments, the chemotherapeutic agent is an agent known to be useful in treating brain cancer.

A number of suitable anticancer agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); agents that bind (e.g., oligomerize or complex) with a Bcl-2 family protein such as Bax; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-a) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-κB modulators; anti-CDK compounds; HDAC inhibitors; proteases; protease inhibitors; and the like. Numerous other examples of chemotherapeutic compounds and anticancer therapies suitable for co-administration with the disclosed compounds are known to those skilled in the art.

In some embodiments, anticancer agents comprise agents that induce or stimulate apoptosis. Agents that induce apoptosis include, but are not limited to, radiation (e.g., X-rays, gamma rays, UV); tumor-derived growth factor ligands, receptors, and analogs; kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, BEXXAR, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine, dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like. In some embodiments, agents are antineoplastic agents including but not limited to temozolomide (Temodar), Carmustine (BiCNU), Cisplatin (Platinol), Erlotinib (Tarceva), Gefitinib (Iressa). In some embodiments, agents of other types may be used, or compositions of the present invention may be co-administered with other agents. In some embodiments, methods of the present invention may be practiced in combination with co-administration of agents of other types. Agents of other types (e.g., for use in systems and compositions of the present invention; for co-administration with systems or compositions of the present invention; for co-administration during the practice of methods of the present invention) include but are not limited to anticonvulsants (e.g., Levetiracetam (Keppra), Phenyloin (Dilantin), Carbamazepine (Tegretol); corticosteroids (e.g., Dexamethasone (Decadron)).

Alkylating agents suitable for use in the present compositions, systems, and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil), floxuridine (fluorode-oxyuridine), and cytarabine (cytosine arabinoside)); and 3) purine analogs (e.g., mercaptopurine (6-mercaptopurine), thioguanine (6-thioguanine), and pentostatin (2′-deoxycoformycin)).

In still further embodiments, chemotherapeutic agents suitable for use in the compositions and methods of the present invention include, but are not limited to: 1) vinca alkaloids (e.g., vinblastine, vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin and carboplatin); 7) anthracenediones (e.g., mitoxantrone); 8) substituted ureas (e.g., hydroxyurea); 9) methylhydrazine derivatives (e.g., procarbazine (N-methylhydrazine)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S. F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1
Exemplary antineoplastic agents.

| | | |
|---|---|---|
| Aldesleukin | Proleukin | Chiron Corp., Emeryville, |
| (des-alanyl-1, serine-125 human interleukin- | | CA |
| 2) | | |
| Alemtuzumab | Campath | Millennium and ILEX |
| (IgG1κ anti CD52 antibody) | | Partners, LP, Cambridge, |
| | | MA |
| Alitretinoin | Panretin | Ligand Pharmaceuticals, |
| (9-cis-retinoic acid) | | Inc., San Diego CA |
| Allopurinol | Zyloprim | GlaxoSmithKline, |
| (1,5-dihydro-4H-pyrazolo[3,4-d]pyrimidin- | | Research Triangle Park, |
| 4-one monosodium salt) | | NC |
| Altretamine | Hexalen | US Bioscience, West |
| (N,N,N′,N′,N″,N″,-hexamethyl-1,3,5- | | Conshohocken, PA |
| triazine-2,4,6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile, a,a,a′,a′- | | Pharmaceuticals, LP, |
| tetramethyl-5-(1H-1,2,4-triazol-1-ylmethyl)) | | Wilmington, DE |
| Arsenic trioxide | Trisenox | Cell Therapeutic, Inc., |
| | | Seattle, WA |
| Asparaginase | Elspar | Merck & Co., Inc., |
| (L-asparagine amidohydrolase, type EC-2) | | Whitehouse Station, NJ |
| BCG Live | TICE BCG | Organon Teknika, Corp., |
| (lyophilized preparation of an attenuated | | Durham, NC |
| strain of Mycobacterium bovis (Bacillus | | |
| Calmette-Gukin [BCG], substrain Montreal) | | |
| bexarotene capsules | Targretin | Ligand Pharmaceuticals |
| (4-[1-(5,6,7,8-tetrahydro-3,5,5,8,8- | | |
| pentamethyl-2-napthalenyl) ethenyl] benzoic | | |
| acid) | | |
| bexarotene gel | Targretin | Ligand Pharmaceuticals |
| Bleomycin | Blenoxane | Bristol-Myers Squibb Co., |
| (cytotoxic glycopeptide antibiotics produced | | NY, NY |
| by Streptomyces verticillus; bleomycin A₂ | | |
| and bleomycin B₂) | | |
| Capecitabine | Xeloda | Roche |
| (5′-deoxy-5-fluoro-N-[(pentyloxy)carbonyl]- | | |
| cytidine) | | |
| Carboplatin | Paraplatin | Bristol-Myers Squibb |
| (platinum, diammine [1,1- | | |
| cyclobutanedicarboxylato(2-)-0,0′]-,(SP-4- | | |
| 2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford Pharmaceuticals, |
| | | Inc., Baltimore, MD |
| Celecoxib | Celebrex | Searle Pharmaceuticals, |
| (as 4-[5-(4-methylphenyl)-3- | | England |
| (trifluoromethyl)-1H-pyrazol-1-yl]benzenesulfonamide) | | |
| Chlorambucil | Leukeran | GlaxoSmithKline |
| (4-[bis(2chlorethyl)amino]benzenebutanoic | | |
| acid) | | |
| Cisplatin | Platinol | Bristol-Myers Squibb |
| (PtCl₂H₆N₂) | | |
| Cladribine | Leustatin, 2-CdA | R.W. Johnson |
| (2-chloro-2′-deoxy-b-D-adenosine) | | Pharmaceutical Research |
| | | Institute, Raritan, NJ |
| Cyclophosphamide | Cytoxan, Neosar | Bristol-Myers Squibb |
| (2-[bis(2-chloroethyl)amino] tetrahydro-2H- | | |
| 13,2-oxazaphosphorine 2-oxide | | |
| monohydrate) | | |
| Cytarabine | Cytosar-U | Pharmacia & Upjohn |
| (1-b-D-Arabinofuranosylcytosine, | | Company |
| C₉H₁₃N₃O₅) | | |
| cytarabine liposomal | DepoCyt | Skye Pharmaceuticals, |
| | | Inc., San Diego, CA |
| Dacarbazine | DTIC-Dome | Bayer AG, Leverkusen, |
| (5-(3,3-dimethyl-1-triazeno)-imidazole-4- | | Germany |
| carboxamide (DTIC)) | | |
| Dactinomycin, actinomycin D | Cosmegen | Merck |
| (actinomycin produced by Streptomyces | | |
| parvullus, C₆₂H₈₆N₁₂O₁₆) | | |
| Darbepoetin alfa | Aranesp | Amgen, Inc., Thousand |
| (recombinant peptide) | | Oaks, CA |
| daunorubicin liposomal | DanuoXome | Nexstar Pharmaceuticals, |
| ((8S-cis)-8-acetyl-10-[(3-amino-2,3,6- | | Inc., Boulder, CO |
| trideoxy-á-L-lyxo-hexopyranosyl)oxy]- | | |
| 7,8,9,10-tetrahydro-6,8,11-trihydroxy-1- | | |
| methoxy-5,12-naphthacenedione | | |
| hydrochloride) | | |
| Daunorubicin HCl, daunomycin | Cerubidine | Wyeth Ayerst, Madison, |
| ((1S,3S)-3-Acetyl-1,2,3,4,6,11-hexahydro- | | NJ |
| 3,5,12-trihydroxy-10-methoxy-6,11-dioxo-1- | | |
| naphthacenyl 3-amino-2,3,6-trideoxy- | | |
| (alpha)-L-lyxo-hexopyranoside | | |
| hydrochloride) | | |
| Denileukin diftitox | Ontak | Seragen, Inc., Hopkinton, |
| (recombinant peptide) | | MA |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis Pharmaceuticals, |
| ((2R,3S)—N-carboxy-3-phenylisoserine, N- | | Inc., Bridgewater, NJ |
| tert-butyl ester, 13-ester with 5b-20-epoxy- | | |
| 12a,4,7b,10b,13a-hexahydroxytax-11-en-9- | | |
| one 4-acetate 2-benzoate, trihydrate) | | |
| Doxorubicin HCl | Adriamycin, Rubex | Pharmacia & Upjohn |
| (8S,10S)-10-[(3-amino-2,3,6-trideoxy-a-L- | | Company |
| lyxo-hexopyranosyl)oxy]-8-glycolyl- | | |
| 7,8,9,10-tetrahydro-6,8,11-trihydroxy-1- | | |
| methoxy-5,12-naphthacenedione | | |
| hydrochloride) | | |
| doxorubicin | Adriamycin PFS | Pharmacia & Upjohn |
| | Intravenous injection | Company |
| doxorubicin liposomal | Doxil | Sequus Pharmaceuticals, |
| | | Inc., Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone injection | Syntex, Corp., Palo Alto, |
| | | CA |
| Elliott's B Solution | Elliott's B Solution | Orphan Medical, Inc |
| Epirubicin | Ellence | Pharmacia & Upjohn |
| ((8S-cis)-10-[(3-amino-2,3,6-trideoxy-a-L- | | Company |
| arabino-hexopyranosyl)oxy]-7,8,9,10- | | |
| tetrahydro-6,8,11-trihydroxy-8- | | |
| (hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, | | Company |
| 3-[bis(2-chloroethyl)carbamate] 17- | | |
| (dihydrogen phosphate), disodium salt, | | |
| monohydrate, or estradiol 3-[bis(2- | | |
| chloroethyl)carbamate] 17-(dihydrogen | | |
| phosphate), disodium salt, monohydrate) | | |
| Etoposide phosphate | Etopophos | Bristol-Myers Squibb |
| (4′-Demethylepipodophyllotoxin 9-[4,6-O- | | |
| (R)-ethylidene-(beta)-D-glucopyranoside], 4′- | | |
| (dihydrogen phosphate)) | | |
| etoposide, VP-16 | Vepesid | Bristol-Myers Squibb |
| (4′-demethylepipodophyllotoxin 9-[4,6-0- | | |
| (R)-ethylidene-(beta)-D-glucopyranoside]) | | |
| Exemestane | Aromasin | Pharmacia & Upjohn |
| (6-methylenandrosta-1,4-diene-3,17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, Inc., |
| (fluorinated nucleotide analog of the antiviral | | Cedar Knolls, NJ |
| agent vidarabine, 9-b-D- | | |
| arabinofuranosyladenine (ara-A)) | | |
| Fluorouracil, 5-FU | Adrucil | ICN Pharmaceuticals, |
| (5-fluoro-2,4(1H,3H)-pyrimidinedione) | | Inc., Humacao, Puerto |
| | | Rico |
| Fulvestrant | Faslodex | IPR Pharmaceuticals, |
| (7-alpha-[9-(4,4,5,5,5-penta | | Guayama, Puerto Rico |
| fluoropentylsulphinyl) nonyl]estra-1,3,5- | | |
| (10)-triene-3,17-beta-diol) | | |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca |
| (acetate salt of [D-Ser(But)⁶,Azgly¹⁰]LHRH; | | Pharmaceuticals |
| pyro-Glu-His-Trp-Ser-Tyr-D-Ser(But)-Leu- | | |
| Arg-Pro-Azgly-NH2 acetate [C₅₉H₈₄N₁₈O₁₄•(C₂H₄O₂)ₓ | | |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal | | |
| antibody Ibritumomab and the linker-chelator | | |
| tiuxetan [N-[2-bis(carboxymethyl)amino]-3- | | |
| (p-isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & Upjohn |
| (5,12-Naphthacenedione, 9-acetyl-7-[(3- | | Company |
| amino-2,3,6-trideoxy-(alpha)-L-lyxo- | | |
| hexopyranosyl)oxy]-7,8,9,10-tetrahydro- | | |
| 6,9,11-trihydroxyhydrochloride, (7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4- | | Switzerland |
| methyl-3-[[4-(3-pyridinyl)-2- | | |
| pyrimidinyl]amino]-phenyl]benzamide | | |
| methanesulfonate) | | |
| Interferon alfa-2a | Roferon-A | Hoffmann-La Roche, Inc., |
| (recombinant peptide) | | Nutley, NJ |
| Interferon alfa-2b | Intron A (Lyophilized | Schering AG, Berlin, |
| (recombinant peptide) | Betaseron) | Germany |
| Irinotecan HCl | Camptosar | Pharmacia & Upjohn |
| ((4S)-4,11-diethyl-4-hydroxy-9-[(4-piperi- | | Company |
| dinopiperidino)carbonyloxy]-1H-pyrano[3′, | | |
| 4′: 6,7] indolizino[1,2-b] quinoline-3,14(4H, | | |
| 12H) dione hydrochloride trihydrate) | | |
| Letrozole | Femara | Novartis |
| (4,4′-(1H-1,2,4-Triazol-1-ylmethylene)dibenzonitrile) | | |
| Leucovorin | Wellcovorin, Leucovorin | Immunex, Corp., Seattle, |
| (L-Glutamic acid, N[4[[(2amino-5-formyl- | | WA |
| 1,4,5,6,7,8 hexahydro4oxo6- | | |
| pteridinyl)methyl]amino]benzoyl], calcium | | |
| salt (1:1)) | | |
| Levamisole HCl | Ergamisol | Janssen Research |
| ((−)-(S)-2,3,5,6-tetrahydro-6-phenylimidazo | | Foundation, Titusville, NJ |
| [2,1-b] thiazole monohydrochloride | | |
| C₁₁H₁₂N₂S•HCl) | | |
| Lomustine | CeeNU | Bristol-Myers Squibb |
| (1-(2-chloro-ethyl)-3-cyclohexyl-1- | | |
| nitrosourea) | | |
| Meclorethamine, nitrogen mustard | Mustargen | Merck |
| (2-chloro-N-(2-chloroethyl)-N- | | |
| methylethanamine hydrochloride) | | |
| Megestrol acetate | Megace | Bristol-Myers Squibb |
| 17α(acetyloxy)-6-methylpregna-4,6- | | |
| diene-3,20-dione | | |
| Melphalan, L-PAM | Alkeran | GlaxoSmithKline |
| (4-[bis(2-chloroethyl) amino]-L- | | |
| phenylalanine) | | |
| Mercaptopurine, 6-MP | Purinethol | GlaxoSmithKline |
| (1,7-dihydro-6H-purine-6-thione | | |
| monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way |
| (9-methoxy-7H-furo[3,2-g][1]-benzopyran-7- | | Exton, Pa |
| one) | | |
| Mitomycin C | Mutamycin | Bristol-Myers Squibb |
| mitomycin C | Mitozytrex | SuperGen, Inc., Dublin, |
| | | CA |
| Mitotane | Lysodren | Bristol-Myers Squibb |
| (1,1-dichloro-2-(o-chlorophenyl)-2-(p- | | |
| chlorophenyl) ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer Ingelheim |
| | | Pharma KG, Germany |
| Oprelvekin | Neumega | Genetics Institute, Inc., |
| (IL-11) | | Alexandria, VA |
| Oxaliplatin | Eloxatin | Sanofi Synthelabo, Inc., |
| (cis-[(1R,2R)-1,2-cyclohexanediamine-N,N′][oxalato(2-)- | | NY, NY |
| O,O′] platinum) | | |
| Paclitaxel | TAXOL | Bristol-Myers Squibb |
| (5β,20-Epoxy-1,2a,4,7β,10β,13a- | | |
| hexahydroxytax-11-en-9-one 4,10-diacetate | | |
| 2-benzoate 13-ester with (2R,3S)—N- | | |
| benzoyl-3-phenylisoserine) | | |
| Pamidronate | Aredia | Novartis |
| (phosphonic acid (3-amino-1- | | |
| hydroxypropylidene) bis-, disodium salt, | | |
| pentahydrate, (APD)) | | |
| Pegademase | Adagen (Pegademase | Enzon Pharmaceuticals, |
| ((monomethoxypolyethylene glycol | Bovine) | Inc., Bridgewater, NJ |
| succinimidyl) 11-17-adenosine deaminase) | | |
| Pegaspargase | Oncaspar | Enzon |
| (monomethoxypolyethylene glycol | | |
| succinimidyl L-asparaginase) | | |
| Pegfilgrastim | Neulasta | Amgen, Inc |
| (covalent conjugate of recombinant | | |
| methionyl human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces | | |
| plicatus) | | |
| Porfimer sodium | Photofrin | QLT Phototherapeutics, |
| | | Inc., Vancouver, |
| | | Canada |
| Procarbazine | Matulane | Sigma Tau |
| (N-isopropyl-μ-(2-methylhydrazino)-p- | | Pharmaceuticals, Inc., |
| toluamide monohydrochloride) | | Gaithersburg, MD |
| Quinacrine | Atabrine | Abbott Labs |
| (6-chloro-9-(1-methyl-4-diethyl-amine)butylamino- | | |
| 2-methoxyacridine) | | |
| Rasburicase | Elitek | Sanofi-Synthelabo, Inc., |
| (recombinant peptide) | | |
| Rituximab | Rituxan | Genentech, Inc., South |
| (recombinant anti-CD20 antibody) | | San Francisco, CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]- | | |
| a(and b)-D-glucopyranose and 220 mg | | |
| citric acid anhydrous) | | |
| Talc | Sclerosol | Bryan, Corp., Woburn, |
| (Mg₃Si₄O₁₀(OH)₂) | | MA |
| Tamoxifen | Nolvadex | AstraZeneca |
| ((Z)2-[4-(1,2-diphenyl-1-butenyl) phenoxy]- | | Pharmaceuticals |
| N,N-dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]- | | |
| as-tetrazine-8-carboxamide) | | |
| teniposide, VM-26 | Vumon | Bristol-Myers Squibb |
| (4′-demethylepipodophyllotoxin 9-[4,6-0- | | |
| (R)-2-thenylidene-(beta)-D- | | |
| glucopyranoside]) | | |
| Testolactone | Teslac | Bristol-Myers Squibb |
| (13-hydroxy-3-oxo-13,17-secoandrosta-1,4- | | |
| dien-17-oic acid [dgr]-lactone) | | |
| Thioguanine, 6-TG | Thioguanine | GlaxoSmithKline |
| (2-amino-1,7-dihydro-6H-purine-6-thione) | | |
| Thiotepa | Thioplex | Immunex Corporation |
| (Aziridine, 1,1′,1″-phosphinothioylidynetris-, | | |
| or Tris (1-aziridinyl) phosphine sulfide) | | |
| Topotecan HCl | Hycamtin | GlaxoSmithKline |
| ((S)-10-[(dimethylamino) methyl]-4-ethyl- | | |
| 4,9-dihydroxy-1H-pyrano[3′,4′:6,7]indolizino | | |
| [1,2-b] quinoline-3,14-(4H,12H)- | | |
| dione monohydrochloride) | | |
| Toremifene | Fareston | Roberts Pharmaceutical |
| (2-(p-[(Z)-4-chloro-1,2-diphenyl-1-butenyl]- | | Corp., Eatontown, NJ |
| phenoxy)-N,N-dimethylethylamine citrate | | |
| (1:1)) | | |
| Tositumomab, I 131 Tositumomab | Bexxar | Corixa Corp., Seattle, WA |
| (recombinant murine immunotherapeutic | | |
| monoclonal IgG₂ₐ lambda anti-CD20 | | |
| antibody (I 131 is a radioimmunotherapeutic | | |
| antibody)) | | |
| Trastuzumab | Herceptin | Genentech, Inc |
| (recombinant monoclonal IgG₁ kappa anti- | | |
| HER2 antibody) | | |
| Tretinoin, ATRA | Vesanoid | Roche |
| (all-trans retinoic acid) | | |
| Uracil Mustard | Uracil Mustard Capsules | Roberts Labs |
| Valrubicin, N-trifluoroacetyladriamycin-14- | Valstar | Anthra --> Medeva |
| valerate | | |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L- | | |
| lyxo-hexopyranosyl]oxyl]-2-naphthacenyl]-2- | | |
| oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R—(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl- | | |
| phosphonoethyl) phosphonic acid | | |
| monohydrate) | | |

Anticancer agents further include compounds which have been identified to have anticancer activity but are not currently approved by the U.S. Food and Drug Administration or other counterpart agencies or are undergoing evaluation for new uses. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCl-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-906-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other therapeutic agents, those skilled in the art are referred to any number of instructive manuals including, but not limited to, the Physician's Desk Reference and to Goodman and Gilman's “Pharmacological Basis of Therapeutics” tenth edition, Eds. Hardman et al., 2001.

In some embodiments, the drug is an siRNA drug (e.g., that targets a tumor specific protein such as ATF5). RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. While the present invention is not limited to any particular mechanism, and an understanding of the mechanism is not necessary to practice the present invention, it is contemplated that following the introduction of a small RNA, or RNAi, into the cell, the sequence is delivered to an enzyme complex called RISC (RNA-induced silencing complex). RISC recognizes the target and cleaves it with an endonuclease. If larger RNA sequences are delivered to a cell, RNase III enzyme (Dicer) converts longer dsRNA into 21-23 nt ds siRNA fragments.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67; herein incorporated by reference in its entirety).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88; each herein incorporated by reference in its entirety). Methods and compositions for performing RNAi with siRNAs are described, for example, in U.S. Pat. No. 6,506,559, herein incorporated by reference in its entirety.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference in their entirety).

While the present invention is not limited to any particular mechanism, and an understanding of the mechanism is not necessary to practice the present invention, it is contemplated that an important factor in the design of siRNAs is the presence of accessible sites for siRNA binding. Bahoia et al., (J. Biol. Chem., 2003; 278: 15991-15997; herein incorporated by reference in its entirety) describe the use of a type of DNA array called a scanning array to find accessible sites in mRNAs for designing effective siRNAs. These arrays comprise oligonucleotides ranging in size from monomers to a certain maximum, usually Corners, synthesized using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, to retain efficacy and target specificity (Sohail et al, Nucleic Acids Res., 2001; 29(10): 2041-2045; herein incorporated by reference in its entirety). Additional methods and concerns for selecting siRNAs are described for example, in WO 05054270, WO05038054A1, WO03070966A2, J Mol. Biol. 2005 May 13; 348(4):883-93, J Mol. Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

In certain embodiments the therapeutic agent (e.g., anti-neoplastic agent) comprises a moiety (e.g., siRNA, antisense RNA, etc.) that specifically and/or preferentially binds to or inhibits a cancer marker or cancer-specific antigen or a nucleic acid (e.g., mRNA transcript) that encodes it. In some embodiments, binding of the cancer marker, tumor-specific agent, or tumor-associated antigen aids in targeting systems and compositions of the present invention to the desired location (e.g., tumor, tumor cells). In some embodiments, the cancer marker, tumor-specific antigen, or tumor-associated antigen is necessary for disease progression (e.g., metastasis, tumor cell proliferation, angiogenesis, and the like) such that binding of the cancer marker, tumor-specific antigen, or tumor-associated antigen affects function of the marker or antigen.

A large number of cancer markers are known to those of skill in the art. Some cell surface components of cancer cells are common to normal cells and others are either qualitatively distinct for or quantitatively increased in tumor cells. Cell surface components common to both normal and malignant cells include, e.g., various kinds of receptors (e.g., certain hormone receptors), histocompatibility antigens, blood group antigens, and differentiation antigens. Receptors include, e.g., sheep erythrocyte receptor, hormone receptors, e.g., estrogen receptor and the like, transferrin receptor, Fc immunoglobulin receptor, nerve growth factor receptor, and the like. Blood group antigens include, e.g., the P determinant and M and N precursor (“T antigen”). Examples of differentiation antigens include, but are not limited to, surface immunoglobulin, and onco-neural antigens. Examples of histocompatibility antigens include, but are not limited to, HLA-A, HLA-B, HLA-DR (Ia-like). In cases where the cell-surface antigen is qualitatively distinct for cancer cells or quantitatively increased in cancer as compared to non-cancer tissues such cell surface markers find use as targets for localizing antibodies.

Antigens that are more restricted to tumor cells include, e.g., inappropriately (ectopically) expressed normal antigens, modified normal antigens, and neoantigens, such as embryonic and fetal antigens, viral antigens, and tumor-specific (or tumor-associated) antigens. Examples of embryonic and fetal antigens include, but are not limited to, fetal onco-neural antigens, onco-fetal antigens, melanoma antigens, colorectal cancer antigens, lung cancer antigens, breast cancer antigens and the like. An example of a virus-associated antigen is the viral capsid antigen of Epstein-Barr virus.

The present invention is not limited by the type of cancer marker, tumor-associated antigen, or tumor-specific antigen. Examples of tumor-specific or tumor-associated antigens include, but are not limited to, CEA, melanoma cell surface antigens, breast cancer cell surface antigens, lung cancer cell surface antigens, colorectal cancer cell surface antigens, gastric cancer cell surface antigens, pancreatic cancer cell surface antigens, glioma cell surface antigens, common sarcoma cell surface antigens, gastrointestinal cancer cell surface antigens, brain tumor cell surface antigens, esophageal cancer cell surface antigens, common epithelial cancer cell surface antigens, osteosarcoma cell surface antigens, fibrosarcoma cell surface antigens, urinary bladder cancer cell surface antigens, prostatic cancer cell surface antigens, renal cancer cell surface antigens, ovarian cancer cell surface antigens, testicular cancer cell surface antigens, endometrial cancer cell surface antigens, cervical cancer cell surface antigens, Hodgkin's disease cell surface antigens, lymphoma cell surface antigens, leukemic cell surface antigens, trophoblastic tumor cell surface antigens, and the like.

Tumor-specific antigens are generally not present on normal cells during any stage of development or differentiation. These may result from mutation of structural genes, abnormal gene transcription or translation, abnormal post-translational modification of proteins, derepression of normally repressed genes, or insertion of genes from other cells or organisms (“transfection”). New tumor-associated antigens may be identified that were previously defined as normal gene products. An antigen need not be tumor-specific to be useful as a target for localizing antibodies used for detection or therapy. For example, an inappropriate receptor may serve as a selective target for antibodies used for cancer detection or therapy.

In various embodiments the markers used in such methods include, but are not limited to MAGE-A3, GalNAcT, MART-1, PAX3, Mitf, TRP-2, and Tyrosinase. Methods for detecting metastatic breast, gastric, pancreas or colon cancer cells can utilize panels of markers such as C-Met, MAGE-A3, Stanniocalcin-1, mammoglobin, HSP27, GalNAcT, CK20, and β-HCG (see, e.g., U.S. Patent Publication 2004/0265845, herein incorporated by reference in its entirety).

In certain embodiments a marker combination of tyrosinase and melanoma-associated antigens MART-1 and MAGE-A3 can be used to detect occult melanoma cells (see, e.g., Bostick et al. (1999) J. Clin. Oncol, 17: 3238-3244).

A wide variety of other cancer markers are known to those of skill in the art. Illustrative cancer markers include, for example, the tumor marker recognized by the ND4 monoclonal antibody. This marker is found on poorly differentiated colorectal cancer, as well as gastrointestinal neuroendocrine tumors (see, e.g., Tobi et al. (1998) Cancer Detection and Prevention, 22(2): 147-152). Human mucins (e.g. MUC1) are known tumor markers as are gp100, tyrosinase, and MAGE, which are found in melanoma. Wild-type Wilms' tumor gene WT1 is expressed at high levels not only in most of acute myelocytic, acute lymphocytic, and chronic myelocytic leukemia, but also in various types of solid tumors including lung cancer. Many kinds of tumor cells display unusual antigens that are either inappropriate for the cell type and/or its environment, or are only normally present during development (e.g. fetal antigens). Examples of such antigens include, but are not limited to, the glycosphingolipid GD2, a disialoganglioside that is normally only expressed at a significant level on the outer surface membranes of neuronal cells, where its exposure to the immune system is limited by the blood-brain barrier. GD2 is expressed on the surfaces of a wide range of tumor cells including neuroblastoma, medulloblastomas, astrocytomas, melanomas, small-cell lung cancer, osteosarcomas and other soft tissue sarcomas.

Other kinds of tumor cells display cell surface receptors that are rare or absent on the surfaces of healthy cells, and which are responsible for activating cellular signaling pathways that cause the unregulated growth and division of the tumor cell. Examples include, but are not limited to, (ErbB2). HER2/neu, a constitutively active cell surface receptor that is produced at abnormally high levels on the surface of breast cancer tumor cells.

Other useful targets include, but are not limited to CD20, CD52, CD33, epidermal growth factor receptor and the like.

In some embodiments, useful targets include brain tumor (e.g., primary brain tumor, e.g., glioblastoma, glioblastoma multiforme) markers, targets, or antigens. Glioblastoma markers, targets, or antigens include but are not limited to ABCC3, GPNMB, NNMT, and SEC61γ (U.S. Pat. No. 7,115,265; herein incorporated by reference in its entirety); neuronal markers (e.g., synaptophysin, neurofilament protein, neuronal nuclear antigen, chromogranin and glial fibrillary acidic protein) (Donev et al. (2010) Neuropath. Applied Neurobiol. 36:411-421; herein incorporated by reference in its entirety); growth arrest and DNA-damage-inducible a (GADD45α), follistatin-like 1 (FSTL1), superoxide dismutase 2, adipocyte enhancer binding protein 1 (Reddy et al. (2008) Clin. Cancer Res. 14:2978; herein incorporated by reference in its entirety); and members of signal transduction pathways including but not limited to the PDGF/PDGFR pathway, the EGF/EGFR pathway, IGF pathway, Ras/Raf/MAPK and PI3K/Akt pathways, angiogenesis pathways (e.g., HIF1 pathway, VEGF pathway), and DNA repair pathways (Palinichamy et al. (2006) Curr. Treatment Options Oncol. 7:490-504; herein incorporated by reference in its entirety).

### II. Therapeutic Applications

As described above, embodiments of the present invention provide methods, systems and compositions for therapeutically targeting brain tumors (e.g., glioblastoma multiforme). Embodiments of the present invention provide methods and systems for using magnetic particles attached to drug-targeting molecule fusions and an external magnetic field. In this way, the drug is specifically targeted to the site of the tumor. In some embodiments, an agent that disrupts the interaction between the drug-targeting molecule and the MION is administered. The drug can then enter the tumor cells and kill or reduce the size of the tumor.

In some embodiments, other types of tumors (e.g., central nervous system tumors, brain tumors, primary brain tumors, secondary brain tumors) are targeted. CNS tumor classes and types include but are not limited to astrocytic, oligodendroglial, oligoastrocytic, ependymal, choroid plexus tumors, other neuroepithelial tumors, neuronal/mixed tumors, pineal tumors, embryonal tumors, Schwannoma, neurofibroma, perineurioma, malignant peripheral nerve sheath tumors, meningioma, mesenchymal tumors, melanocytic lesions, lymphomas and hematopoietic neoplasms, germinoma, glioblastoma, teratoma, craniopharyngioma, granular cell tumors, metastatic tumors, oligodendrogliomas, fibrillary astrocytomas.

In some embodiments, MION-drug-targeting conjugates are administered intravenously. In other embodiments, they are administered intra-arterially. In some embodiments, in order to maintain intact blood flow at the site of arterial injection, a catherization technique that does not require vessel occlusion is used. For example, in some embodiments, a think needle or capillary is used to inject therapeutic compositions. In some embodiments, administration is intranasal administration.

As described above, any number of drugs and targeting agents are suitable for use in embodiments of the present invention. Dosages and timing of administration are well known to those in the art and are generally determined experimentally (e.g., using the methods described in Examples 2 and 3 below).

Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

### III. Diagnostic Applications

In some embodiments, the present invention provides compositions and methods for diagnostic applications. For example, in some embodiments, diagnostic applications are used to image and define brain tumor boundaries. Such images find use, for example, prior to and during surgical removal of brain tumors. The compositions and methods described above are suitable for use in diagnostic applications. In some embodiments, the therapeutic component (e.g., anti-cancer drug) is removed from the nanoparticle. In some embodiments, therapeutic components of nanoparticles are replaced with imaging components.

In some embodiments, nanoparticles comprise contrast agent for imaging (e.g., X-Ray, computer tomography (CT) imaging, or MRI imaging). In some embodiments, nanoparticles comprise imaging targeting moieties (e.g., nucleic acids, PNAs, peptides, proteins, antibodies, etc.) that target the conjugates to a region of interest (e.g., tumor).

In some embodiments, nanoparticles are used in research (e.g., imaging in animal models, structural studies, DNA-protein binding interactions, protein capture, etc.) or drug screening applications.

### IV. Kits and Systems

In some embodiments, the present invention provides kits for using in research, diagnostic and therapeutic applications. In some embodiments, kits include components necessary, sufficient or useful in performing the methods of embodiments of the present invention.

In some embodiments, kits include drug-targeting molecule compositions, MIONs, molecules for removing the drug compositions from the MIONs, along with any controls, buffers, reagents, administration tools, etc.

Kits may further comprise appropriate controls and/or detection reagents. Any one or more reagents that find use in any of the methods described herein may be provided in the kit.

In some embodiments, the present invention provides systems for use in targeting and treating brain tumors. In some embodiments, systems comprise the above described components and a device for generating an external magnetic field for orienting MIONs at the tumor site (e.g., magnets, electromagnets, etc.). Other suitable devices for generating magnetic fields are within the scope of one of skill in the art.

### EXPERIMENTAL

The following examples are provided in order to demonstrate and further illustrate certain preferred embodiments and aspects of the present invention and are not to be construed as limiting the scope thereof.

### Example 1

### MRI-Monitored Magnetic Targeting

MIONs with different coatings (starch, dextran) were purchased from Chemicell (Berlin, Germany) and also self-synthesized using a modified protocol of Palmacci and Josephson (U.S. Pat. No. 5,262,176; herein incorporated by reference in its entirety). MION thus prepared exhibited superparamagnetic behavior, narrowly distributed hydrodynamic diameter (110±22 nm), and a saturation magnetization (Ms) of 94 emu/g Fe. Magnetic targeting and MRI were performed according to a previously published procedure (Kwon et al., Expert Opin. Drug Deliv. 2008; 5:1255-1266; herein incorporated by reference in its entirety). For magnetic targeting, animals harboring 9L glioma were anesthetized and placed ventrally on a platform with their heads positioned between the poles of an electromagnet. The magnetic field density was adjusted to 0 T for non-targeted animals (i.e. without magnetic targeting) or 0.4 T for targeted animals. Animals were injected intravenously or intra-arterially with a MION suspension at a dose of 12 mg Fe/kg, and retained in the magnetic field for 30 min. MRI was conducted using a 12-cm horizontal-bore, 7 Tesla Unity Inova imaging system (Varian, Palo Alto, Calif.). A single-slice gradient echo sagittal image was acquired to facilitate reproducible positioning of the animal head within the magnet coil using the base of the skull as a reference point. The time course of MION distribution in the rat brain was monitored by serial acquisition of gradient echo (GE) and T2-weighted MRI scans. Images were acquired before MION administration (pre-scans) and after magnetic targeting at about 45 min intervals over a 4 hour period.

Magnetic Targeting via Intravenous (I.V.) Administration

Passive targeting of a brain tumor via the EPR effect is often compromised by the rapid plasma clearance of the nano-carriers. To examine the benefits of utilizing MION as the drug carrier in achieving magnetic-mediated active targeting and MRI visibility of the brain tumor, starch-coated MION (referred to as G-100) was injected via the tail vein into the glioma-bearing rats.

Visualization of Brian Tumor Targeting via MRI

FIG. 2 presents a subset of a typical series of MRI images obtained from magnetically (A) targeted; and (B) non-targeted animals before and after i.v. injection of MION. The brain tumors were clearly visible on the baseline T2-weighted images in both animal groups. As seen in FIG. 2A, the GE images of the targeted animal acquired 1 and 3 hours post injection (0.5 and 2.5 hrs post magnetic targeting, respectively) exhibited a region of pronounced hypointensity compared to the baseline GE image. This hypointense region indicated the presence of magnetic nanoparticles within the tumor tissue. In contrast, the post-injection images of the non-targeted animal (FIG. 2B) displayed no detectable signal reduction within the glioma lesion. These results indicated that MION accumulation in the tumor by conventional passive targeting via EPR was nearly negligible. However, with use of magnetic targeting, both the accumulation and retention of MION in the tumor were vastly improved to a degree that was clearly visible by MRI.

Quantification of MION Accumulation by MRI

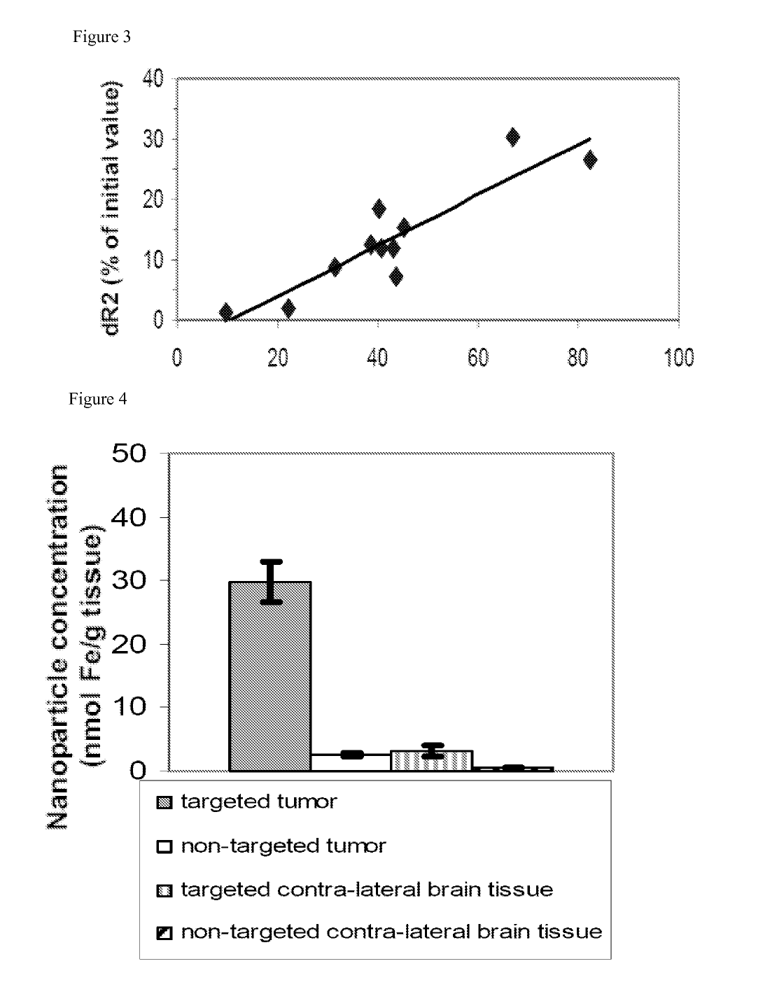

To quantitatively determine MION accumulation in the brain, information from R2 relaxivity maps of the animals is analyzed, assuming that the change in relaxivity relative to the pre-scan (dR2) is dictated by the change in MION concentration. Briefly, the R2 values used to analyze the time course of R2 relaxivity change after MION administration were obtained from the mean signal intensity within the defined regions of interest (ROI; manually drawn in the tumor lesion and contralateral normal brain) on the R2 relaxivity maps. The change in R2 relaxivity caused by the presence of the contrast agent (i.e. MION) within the tissue of interest at time t (i.e. dR2(%)), expressed as percentage change of the initial (pre-scan; t=0) relaxivity value according to the equation dR2(%)={[R2(t)−R2(0)]/R2(0)}×100%, would correlate to MION concentration in the tissue at the specific time t. To verify the results, dR2 changes measured in vivo using MRI were compared to MION concentrations precisely quantified ex vivo by using the ESR method. ESR has been used widely to directly quantify the presence of paramagnetic species. As shown in FIG. 3, the ESR results obtained from the excised tumors were found to be linearly correlated (slope=0.57 g tissue/nmol Fe, p=0.0001, R2=0.88) with the dR2 data acquired from the MRI image map.

Targeting Efficiency & Selectivity via Intravenous Administration

FIG. 4 shows that MION concentrations determined by ESR showed that i.v. administration along with magnetic targeting resulted in an 11.5-fold increase (p<0.0005) in MION accumulation in tumors with magnetic targeting (Column #1 from left; 29.8±7.9 nmol Fe/g tissue) over that of the tumors without magnetic targeting (Column #2 from left; 2.6±0.7 nmol Fe/g tissue); indicating a markedly enhanced efficiency in capture and retention of MION by the tumor via application of an external magnetic field. A 9.5-fold difference (p<0.0005) in the MION concentration between the tumors (29.8±7.9 nmol Fe/g tissue; Column #1) and contra-lateral normal brain (3.1±2.1 nmol Fe/g tissue; Column #3 from left) of animals with magnetic targeting was observed, indicated a greatly distinct targeting selectivity on brain tumor over the contra-lateral normal brain. The marginal difference in MION accumulation between tumors and normal brain tissues of the animals without magnetic targeting (2.6±1.4 vs. 0.5±0.1 nmol Fe/g tissue; Column #2 from left vs. Column #4) indicated that contribution of the EPR effect to tumor retention of MION by passive targeting was relatively minimal, despite the pronounced differences in vascular permeability between the intact blood-brain barrier (BBB) and the compromised blood tumor barrier.

Magnetic Targeting via Intra-arterial (LA.) Administration

Despite the 11.5-fold increase in the total glioma exposure to MION by magnetic targeting over the non-targeted tumor tissues, the overall MION accumulation in the tumor (i.e. ˜30 nmol Fe/g tissue) was still below 0.01% of the initially administered dose (12 mg Fe/kg); a finding consistent with that documented in the literature (Enochs et al., J Mag Reson Imaging. 1999; 9:228-32; Kim and Shima, J. Appl. Phys. 2007; 101: 09M516; herein incorporated by reference in its entirety). While the present invention is not limited to any particular mechanism, and an understanding of the mechanism is not necessary to practice the present invention, it is contemplated that this was primarily due to rapid renal and hepatic clearance of MION, particularly of those with positive charges. It was reported that polylysine-coated MION had a plasma half-life of merely 1-2 minutes (Papisov et al., J. Magnetism Magn. Mater. 1993; 122:383-386; herein incorporated by reference in its entirety). Intra-arterial administration offers the advantage of allowing carriers to bypass renal and hepatic clearance during their first passage through the circulation, and was therefore exploited to further enhance brain tumor magnetic targeting. While the present invention is not limited to any particular mechanism, and an understanding of the mechanism is not necessary to practice the present invention, it is contemplated that with recent advances in endovascular technology and the increased safety of angiographic procedures, carotid administration of nanoparticles has become a clinically-relevant alternative to intravenous injection (Lubbe et al., Cancer Res. 1996; 56:4694-701; herein incorporated by reference in its entirety).

Consistent with previous studies (Lubbe et al., Cancer Res. 1996; 56:4694-4701; herein incorporated by reference in its entirety), it was found that when mice were injected with high concentrations of MION, significant embolism of the afferent vasculature occurred due to their aggregation. While the present invention is not limited to any particular mechanism, and an understanding of the mechanism is not necessary to practice the present invention, it is contemplated that occlusion of the carotid artery, which directly supplies the normal brain parenchyma, can lead to severe neuro-sequelae (Yoshizaki et al., Exp. Neurol. 2008; 210:585-591; herein incorporated by reference in its entirety) and is generally avoided. Also, blockage of the blood flow hinders the convective transport of MION towards the tumor site. Magnetic field topography and strength have been shown the capability in modulating the extent of MION aggregation arterial and capillary blood flow rates (Driscoll et al., Microvasc. Res. 1984; 27:353-369; herein incorporated by reference in its entirety).

MRI-Guided Optimization of Magnetic Field Topography to Abolish Vascular Embolism

During experiments conducted during the course of developing certain embodiments of the present invention, magnetic targeting strategy was designed by optimizing the arterial flow dynamics and magnetic field topography to override the vascular embolism obstacle. To maintain carotid flow dynamics, a new catheterization procedure that would not require vessel occlusion was used that comprises inserting a thin silica capillary tubing (OD˜150 μm), which functioned as a needle, through the vascular wall without compromising the integrity of the artery. This catheterization technique did not impede the blood flow through the artery, and allowed infusion of MION suspension at a rate of 5 μL/sec, while maintaining intact arterial hydrodynamics.

To reduce the exposure of MION in the afferent arterial vasculature to magnetic force, an optimized magnet configuration was used. The approach, shown in FIG. 5B, stemming from the outcomes of magnetic field simulations, involved mounting of a small cylindrical permanent magnet (d=9 mm) to a tapered pole of a standard (FIG. 5A) dipole electromagnet. This configuration served to divert the magnetic flux lines emanating from the electromagnet poles to pass through the low resistance cylindrical attachment, thus generating a local maximum of the magnetic field on the exposed pole magnetic flux density. Mapping of the magnetic flux density revealed that the dipole electromagnet, which was used previously for brain targeting studies (Park et al. FASEB J. 2005; 19:1555-1557; herein incorporated by reference in its entirety), generated broad-range and shallow-gradient topography (FIG. 5C). In contrast, the topographic map obtained with the optimized magnet configuration (FIG. 5D) exhibited a focal region of maximal flux density (350 mT) which decayed rapidly with the distance from the peak. The small dimensions of the cylindrical attachment allowed for focusing the magnetic flux density over a narrow circular region (d ˜5 mm) approximating to the cross-section of the tumor lesion.

As seen in FIG. 5F, by applying this new magnetic setup with optimized magnetic field topography, a 6-fold reduction of the magnetic force at the injection site was acheived, alleviating MION aggregation in the afferent vasculature observed previous (FIG. 5E).

To fully utilize the benefits of the magnetic field topography, the next step was to align the tumor lesion with the region of the maximal magnetic flux density by properly positioning the rat with respect to the magnetic setup. For such positioning, MRI was used to determine the intracerebral localization of the tumor lesion, since the tumor could be clearly visualized as a hyperintense region on T2-weighted MRI scans. In general, acquisition of 12 axial slices of the animal head allowed mapping the location of the tumor lesion with respect to such externally visible anatomical features of the animal head as the center of the eye and the midline of the head. Using the MRI-derived tumor coordinates the rat was positioned in a way that maximized the exposure of the tumor lesion to the magnetic force, while at the same time minimizing the exposure of the afferent vasculature.

Targeting Efficiency/Selectivity via Intra-arterial Administration

To assess the effects of the system on targeting efficacy, experiments were conducted using both intravenous and intra-arterial administration of polyethyleneimine (PEI)-coated MION (termed PEI-MION). PEI-MION was specifically chosen as the test model because with its highly positive surface the plasma clearance was found to be 8-fold faster than that of starch-coated MION (G100). This exceedingly short plasma half-life rendered PEI-MION capable of elucidating the beneficial effects of magnetic targeting by the intra-arterial route.

Materials and Methods for these studies are detailed infra.

Materials

Iron oxide nanoparticles, coated with starch or gum arabic polysaccharide matrix, were used (Chemicell Berlin, Germany). These particles are referred to as G100 and Gara, respectively. Low molecular weight polyethyleneimine (PEI, M_{w}w1200) was purchased from Sigma-Aldrich. 1-Ethyl-3-(3-dimethylaminopropyl) carbodiimide hydrochloride (EDC) and N-hydroxysulfosuccinimide

(sulfo-NHS) were obtained from Pierce (Rockford, Ill., USA).

Preparation and Characterization of PEI-modified Nanoparticles

Surface modification of carboxyl-bearing Gara with primary amine-containing PEI was carried out using EDC coupling chemistry (Holzapfel et al. (2006) J. Phys. Condens. Matter 18:S2581-2594; herein incorporated by reference in its entirety). Gara nanoparticles were mixed with PEI, EDC and sulfo-NHS at a molar ratio of 1(Fe):1:2:2 (pH=6). This reaction mixture was incubated at room temperature for 48 h. Mixtures of nanoparticles and PEI, without activating reagents, were employed as controls. The modified nanoparticles were purified on a magnetic separator, with deionized water, until the presence of free amines in the supernatant could no longer be detected with the ninhydrin assay, described below. The resulting nanoparticles, bearing pendant PEI chains, were termed GPEI.

Quantification of primary and secondary amines on the nanoparticle surface was carried out by ninhydrin colorimetric assay (Kaiser et al. (1970) Anal. Biochem. 34:595-598; herein incorporated by reference in its entirety). The ninhydrin reagent (500 μL of 0.2% w/v in 0.1 M buffer phosphate, pH 9) was added to 200 μl of nanoparticle sample and the mixture heated in a boiling water bath for 15 min. Samples were cooled to room temperature and placed on a magnetic separator to remove the nanoparticles. The absorbance of supernatants was measured at 570 nm on a microplate reader (Power-Wave 340, Bio-Tek Instruments, Winooski, Vt.) and the amine content quantified using ethanolamine standard curves.

Zeta potential and hydrodynamic diameter of purified GPEI and Gara were measured on a PSS Nicomp 380/ZLS Zeta Potential and Submicron Particle Size Analyzer (Nicomp, Santa Barbara, Calif.). Magnetization measurements of aqueous nanoparticle preparations were performed at 293 K using a MPMS-XL Superconducting Quantum Interference Device (SQUID) magnetometer (Quantum Design Inc. San Diego, Calif.). Iron concentrations of nanoparticle preparations were determined by Inductively Coupled Plasma—Optical Emission Spectroscopy (ICP-OES) using an Optima 2000 DV spectrometer (PerkineElmer, Boston Mass.) (Chertok et al. (2008) 29:487-496; herein incorporated by reference in its entirety).

Cell Uptake Assay

Rat 9L-glioma cells were seeded in a 6-well plate. Cells (1.5×10⁵ cells/well) in Dulbecco's Modified Eagle's Medium (DMEM) supplemented with 10% heat-inactivated fetal bovine serum (FBS), 100 IU/ml penicillin, 100 μg/ml streptomycin and 0.29 mg of L-glutamine (complete medium, 2 ml) were allowed to attach overnight at 37° C. in a humidified atmosphere of 5% CO₂. The medium was then carefully aspirated and substituted with 2 ml of serum-free DMEM (control) or serum-free DMEM containing G100 or GPEI nanoparticles at a concentration of 45 μg Fe/ml (test). Cells were incubated at 37° C. for 1 h, washed three times with serum-free DMEM to remove unbound nanoparticles and further incubated overnight with complete medium. For qualitative analysis, the medium was replaced with PBS and images of the cells were acquired with a digital camera using phase contrast microscopy. For quantitative analysis, the cells were harvested with 0.5 ml trypsin-EDTA (0.25% trypsin, 1 mM EDTA) and complete medium added to inhibit trypsin. The cells were counted in a hemacytometer and washed by five cycles of dispersion in PBS, centrifugation and supernatant removal. After the last centrifugation and aspiration of the supernatant, cells were resuspended in 100 mL of PBS, transferred to ESR (electron spin resonance) tubes and kept at −80° C. until analysis.

Assay for Nanoparticle Cytotoxicity

The cytotoxicity of nanoparticles to 9L-glioma cells was assessed with the MTT (3-[4,5-dimethyl-thiazol-2-yl]-2,5-diphenyltetrazolium bromide) cell viability assay (Mosmann (1983) J. Immunol. Meth. 65:55-63) with minor modification as described herein. 9L cells were seeded in a 96-well plate at a density of 2×10³ cells/well in 100 mL DMEM complete medium and allowed to adhere overnight at 37° C. in a humidified atmosphere of 5% CO₂. The medium was then replaced with 100 μL of either serum-free DMEM (control) or serum-free DMEM containing G100 or GPEI nanoparticles at a concentration of 45 μg Fe/ml (test). Cells were incubated for 3 h at 37° C., then washed with fresh DMEM and further incubated overnight with complete medium. Since phenol red was found to interfere with the MTT assay, DMEM not containing phenol red was used for the overnight incubation step (Invitrogen, 21063-029). MTT solution (20 μL of 6 mg/ml in PBS) was added to each well and cells were incubated for 2 h at 37° C. and 5% CO₂. The medium was then carefully replaced with 100 μL DMSO and the plates were incubated for 30 min at room temperature to dissolve formazan crystals. The absorbance was measured at 550 nm using a microplate reader (Power-Wave 340, Bio-Tek instruments, Winooski, Vt.). To calculate the number of cells from the measured absorbance values, a calibration curve was constructed with known 9L cell concentrations, counted with a hemacytometer. The calibration curve was found to be linear within a range of 2×10³-6×10⁴ 9L cells.

In Vivo Studies

Pharmacokinetic Analysis

The pharmacokinetics of G100 and GPEI magnetic nanoparticles were studied in male Fisher 344 rats weighting 200-250 g. The nanoparticles were administered intravenously via tail vein and the blood was sampled through the cannulated carotid artery. The animals were anaesthetized by intraperitoneal injection of ketamine/xylazine mixture (87/13 mg/kg body weight). The left carotid artery of the animals was exposed by blunt dissection and ligated rostrally to occlude the flow.

PE-10 tubing was then inserted caudally via a small incision in the arterial wall and secured in place by ligation. The intra-carotid catheter was flushed with Heparin flush solution (Hepflush-10, 10 USP Units/ml, Abraxis Pharmaceutical Products, IL) and clamped. Tail veins of the animals were cannulated with a 26-gauge angiocatheter (Angiocath™ catheter, Becton Dickinson, Sandy, Utah). The nanoparticle suspension in PBS was administered to rats via the tail vein catheter at a dose of 12 mg Fe/kg body weight. Blood samples of 100 μl, were collected from the cannulated carotid artery in 0.5 ml Eppendorf tubes spiked with 10 μL of Heparin solution (5000 USP Units/ml).

Samples were acquired before and serially after nanoparticle administration at preset time intervals over 60 min duration. Plasma fractions were immediately separated by centrifugation (3 min at 7000 g) and stored at −80° C. until analysis by ESR, detailed infra.

Induction of Brain Tumors

Intracerebral 9L tumors were induced in male Fisher 344 rats weighing 125-150 g (Ross et al. (1998) PNAS 95:7012-7017; herein incorporated by reference in its entirety). Rat 9L-glioma cells were cultured in Dulbecco's modified Eagle's medium (DMEM) supplemented with 10% heat-inactivated fetal bovine serum, 100 IU/ml penicillin, 100 μg/ml streptomycin and 0.29 mg of L-glutamine at 37° C. in a humidified atmosphere of 5% CO₂. Prior to implantation, cells were grown to confluency in 100 mm culture dishes, harvested and resuspended in serum-free DMEM at a concentration of ˜10⁵ cells/pt. The cell suspension (10 μL) was implanted in the right forebrain of the animals at a depth of 3 mm beneath the skull through a 1-mm-diameter burr hole. The surgical field was cleaned with 70% ethanol and the burr hole was filled with bone wax (Ethicon Inc., Summerfield, N.J.) to prevent extracerebral extension of the tumor. The tumor volume of the animals was monitored with MRI beginning on day 10 after cell implantation to select tumors between 70 and 90 μL, for magnetic targeting experiments.

Magnetic Resonance Imaging (MRI) Studies

MRI experiments were performed on an 18-cm horizontal-bore, 7 T Varian Unity Inova imaging system (Varian, Palo Alto, Calif.). Animals were anesthetized with 1.5% isofluorane/air mixture and imaged using a 35-mm-diameter quadrature RF head coil (USA Instruments Inc, OH). Animals were maintained at 37° C. inside the magnet using a thermostated circulating water bath. To visualize the tumor localization within the rat brain, 13 axial sections of the brain were acquired with a T₂-weighted fast spin echo sequence using the following parameters: repetition time (TR)=4 s, echo time (TE)=60 ms, field of view=30×30 over 128×128 matrix, slice thickness=1 mm, slice separation=2 mm, four signal averages per phase encoding step.

To determine nanoparticle distribution in the brain, 13 gradient echo (GE) axial slices of the brain were collected before the nanoparticle administration (baseline scans) and immediately following magnetic targeting. GE images were acquired with the following parameters: TR=20 ms, TE=5 ms, field of view=30×30 over 128×128 matrix, slice thickness=1 mm.

Magnetic Targeting

Magnetic targeting studies were carried out in tumor-bearing rats using either the intravenous or intra-arterial route for nanoparticle administration. For intravenous administration, the tail vein was cannulated with a 26-gauge angiocatheter (Angiocath™, Becton Dickinson, Sandy, Utah). For intra-arterial administration, the right carotid artery was cannulated (Rodriguez e tal. (1992) Physiol. BBehay. 52:1211-1213; herein incorporated by reference in its entirety).

The magnetic setup was optimized to achieve sharp-gradient of magnetic flux density at the target location. Briefly, a small cylindrical neodymium-iron-boron magnet (NdFeB, 9 mm in diameter) was attached to the pole of a dipole electromagnet (GMW associates, Model 3470). The animals were placed supinely on a platform with their heads positioned directly on the pole face of the small magnet. The magnetic field density at the pole face of the small magnet was adjusted to 350 mT. Animals were then injected with nanoparticle suspension at a dose of 12 mg Fe/kg through either an intravenous or intra-carotid catheter, and retained in the magnetic field for 30 min. The rats were imaged with MRI before the administration of nanoparticles and after the magnetic targeting as described herein. Immediately following MRI, the animals were sacrificed, dissected and the isolated brain divided into right and left hemispheres. The tumor was carefully separated from the normal tissue of the right hemisphere. The left hemisphere and tumor tissues were frozen and kept at −80° C. until analysis by ESR.

Ex Vivo Analysis of Tissue and Plasma Samples by Electron Spin Resonance (ESR) Spectroscopy

Nanoparticle concentrations were determined by ESR (Chertok et al. (2010) Mol. Pharmaceutics 7:375-385; herein incorporated by reference in its entirety). ESR spectra of the samples were acquired using an EMX ESR spectrometer (Bruker Instruments Inc., Billerica, Mass.) equipped with a liquid nitrogen cryostat. The acquisition parameters were: resonant frequency: ˜9.2 GHz, microwave power: 20 mW, temperature: 145 K, modulation amplitude: 5 G and receiver gain of 5×10⁴ and 5×10³ fortissue and plasma samples, respectively.

Due to the 100 kHz modulation of the magnetic field, the measured signal was the derivative δP/δH of the absorbed microwave power with respect to the external static field H. The integral ∫P(H)dH is known to be proportional to the amount of resonating electronic spins present in the sample. Therefore, the double integral of the ESR spectra of samples was calculated to quantify the nanoparticles. Calibration curves were constructed with nanoparticle solutions of known iron concentrations. The data were corrected for background tissue absorption using control tissue samples from animals not exposed to nanoparticles or control plasma samples collected prior to nanoparticle injection.

Quantitative Data Analysis

The tumor delivery advantage, R_{d}, resulting from arterial nanoparticle administration as compared to the intravenous route was calculated by a previously described equation (Eckman et al. (1974) J. Pharmacokinet Biopharm. 2:257-285; herein incorporated by reference in its entirety):

$$\text{?} = ( ) / ( \text{?} ) \\ \text{?} \text{indicates text missing or illegible when filed} \qquad ( Equation 1 )$$

where D is the dose, F the blood flow through the infused artery, and CO the cardiac output; Iᵣₐ and Iᵣᵥ are the arterial AUCs for the systemic recirculation of the substance after arterial and venous administration, respectively.

The AUC was estimated numerically by a linear trapezoidal integration method; integration was performed on the interval of 0-30 min after nanoparticle administration, corresponding to the duration of magnetic targeting.

Statistical Analysis

Data are presented as mean±SE, unless indicated otherwise. SPSS 15.0 statistical software package (SPSS Inc., Chicago, Ill.) was used for data analysis. Group means were compared using an unpaired two-tailed t-test (experiments comparing two groups of cells/animals) or one-way ANOVA (experiment comparing 3 groups of cells). The assumption of the homogeneity of variances was assessed using Levene's test. Statistical difference within data sets (two groups) that were found to violate the assumption of the homogeneity of variances was inferred by Welch-Satterthwaite t-test not assuming equal variances. A p-value of <0.05 was considered statistically significant.

Results

GPEI Formation and Characterization

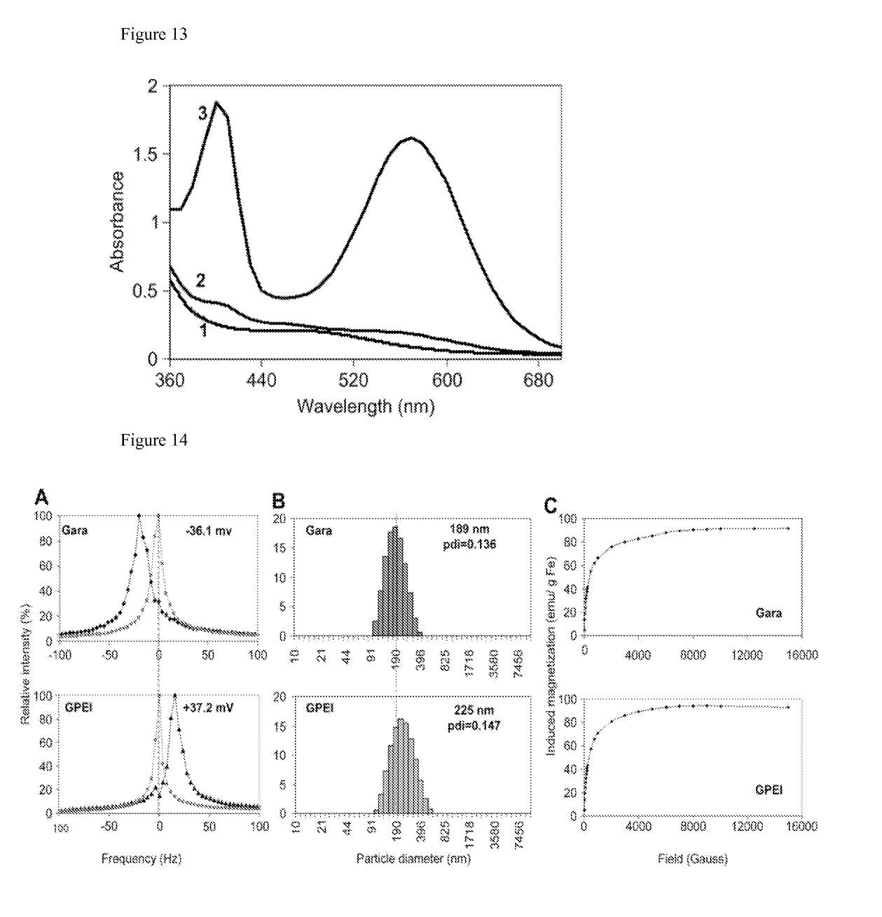

GPEI nanoparticles were prepared by covalent grafting of low molecular weight branched PEI chains onto the surface of Gara. Since primary and secondary amines constitute about 75% of all PEI amino groups (McBain et al. (2007) 17:2561-2565; herein incorporated by reference in its entirety), the success of PEI surface grafting can be readily assessed with the ninhydrin assay (Friedman (2004) J. Agric. Food Chem. 52:385-406; herein incorporated by reference in its entirety). Ninhydrin reacts with immobilized amines to generate a soluble Ruhemann's purple chromophore, which can be detected in the supernatant (Kaiser et al. (1970) Anal. Biochem. 34:595-598; herein incorporated by reference in its entirety). The absorbance of the Ruhemann's complex at 570 nm is proportional to the surface amine density. FIG. 13 shows that, following thorough purification, covalently modified GPEI nanoparticles exhibited 14-fold higher absorbance at 570 nm than the physical mixture of Gara and PEI at the same nanoparticle concentration. Since no loss of amine groups was detected with additional purification of GPEI, the high amine content of the GPEI surface can be attributed to covalent PEI grafting and not to physical surface adsorption.

Grafting of PEI to the surface of amine-bearing magnetic nanoparticles was previously accomplished with a two-step procedure, requiring glutaraldehyde as a crosslinker (McBain et al. (2007) J. Meter. Chem. 17:2561-2565; herein incorporated by reference in its entirety). PEI chains were attached directly to carboxyl-bearing nanoparticle surface via a one-step EDC coupling chemistry. A previously reported procedure resulted in nitrogen content of 0.635 μmol per mg of nanoparticles, corresponding to about 15.5 nmol PEI (MW˜1800 Da) per mg of nanoparticles. In experiments detailed herein, the amine density on the nanoparticle surface was estimated to be 0.437 μmol/mg nanoparticles. This amine content corresponds to about 15.6 nmol PEI (MW˜1200 Da) per mg of nanoparticles. Thus, in spite of the simplified procedure employed, the PEI grafting efficiency was comparable to that achieved in the previous study.

Further physical characterization of GPEI nanoparticles included evaluation of 4-potential, size distribution and magnetic properties. Measurement of 4-potential served to further corroborate the success of surface PEI grafting. At a pH of 5.5, the ζ-potential of Gara was measured to be −36.1 mV. This negative z-potential can be ascribed to the high content of glucoronic acid found in Gara surface coating as reported by the manufacturer. In contrast, GPEI exhibited a positive ζ-potential of +37.2 mV at the same pH. The shift in ζ-potential (FIG. 14A) can be attributed to the PEI graft, which provided GPEI surface with a high density of protonable amine groups. Mean hydrodynamic diameter of the nanoparticles was found to increase slightly from 189 nm for Gara to 225 nm for GPEI due to PEI grafting (FIG. 14B). Yet, GPEI still exhibited monomodal size distribution with a polydispersity index (PDI) of 0.147, comparable to that of the starting material Gara (PDI=0.136). Measurement of induced magnetization by SQUID magnetometry showed no significant alteration in nanoparticle magnetic properties due to surface PEI grafting (FIG. 14C). Magnetization curves exhibited no hysteresis and no remanent magnetization, consistent with superparamagnetic behavior. Saturation magnetization of GPEI was found to be 93 emu/g Fe, indicating that GPEI is amenable to magnetic targeting.

The in vitro and the in vivo behavior of the GPEI nanoparticles were assessed and compared to that of commercially available starch-coated magnetic nanoparticles (G100). G100 nanoparticles have been extensively studied in magnetic targeting applications. The physical properties of G100 nanoparticles are summarized in Table 2.

TABLE 2
Physical properties of G100 magnetic nanoparticles.
Data expressed as Mean ± SD.

| Property | Value | Units | Reference |
|---|---|---|---|
| Hydrodynamic diameter | 110 (±22) | nm | [13] |
| Saturation magnetization of an | 125 | Emu/g Fe | Present study |
| aqueous suspension (Ms) | | | |
| R₂ relaxivity | 43.8 (±2.6) | s⁻¹mM⁻¹ | [13] |
| ζ-potential | −12 (±2) | mV | Present study |

Cell Uptake and Cytotoxicity

Many therapeutic agents exert their pharmacological action inside the cell. Therefore, while the present invention is not limited to any particular mechanism, and an understanding of the mechanism is not necessary to practice the present invention, it is contemplated that the ability of a nanocarrier to internalize into cells can augment its drug delivery performance. The interaction of GPE1 and G100 nanoparticles with 9L-glioma cells was assessed qualitatively with optical microscopy and quantitatively with ESR spectroscopy.

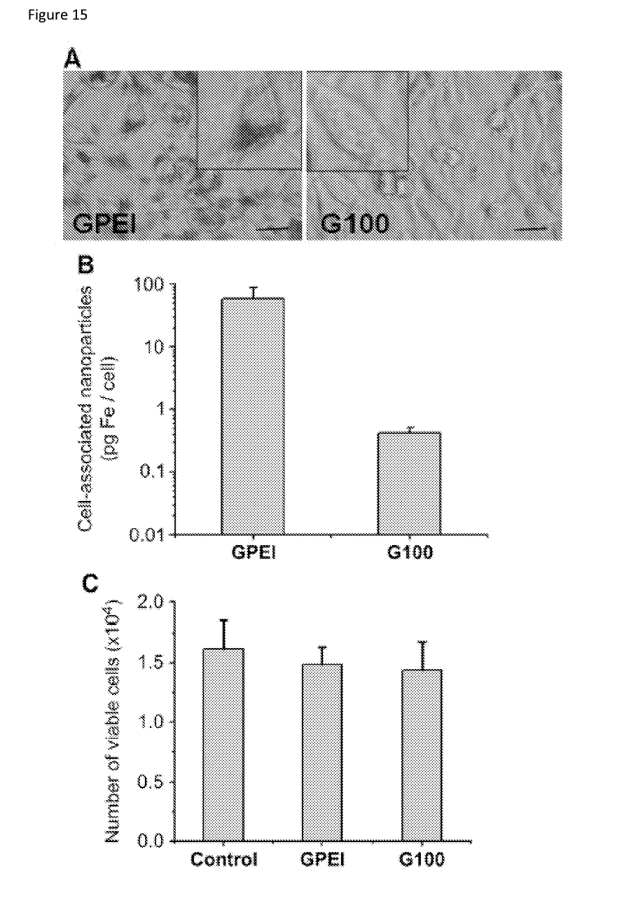

Optical micrographs, obtained after nanoparticle incubation with 9L-glioma cells and extensive washing, revealed appearance of dark pigmentation in the GPEI-exposed but not the G100-exposed cells (FIG. 15A). This pigmentation qualitatively indicated enhanced cellular uptake of GPEI compared to G100. Quantitative analysis further demonstrated that the extent of cellular uptake was 138-fold higher (p=0.013, FIG. 15) for GPEI (58±17 pg Fe/cell) than for the G100 (0.42±0.05 pg Fe/cell). Despite the significantly enhanced cellular uptake, the polycationic nanoparticle surface did not increase cell toxicity compared to the slightly anionic G100, as MTT cell viability test revealed no significant difference (p=0.712) between the GPEI treated, G100-treated and untreated control cells (FIG. 15C).

Magnetic Targeting of GPEI with Intravenous Administration

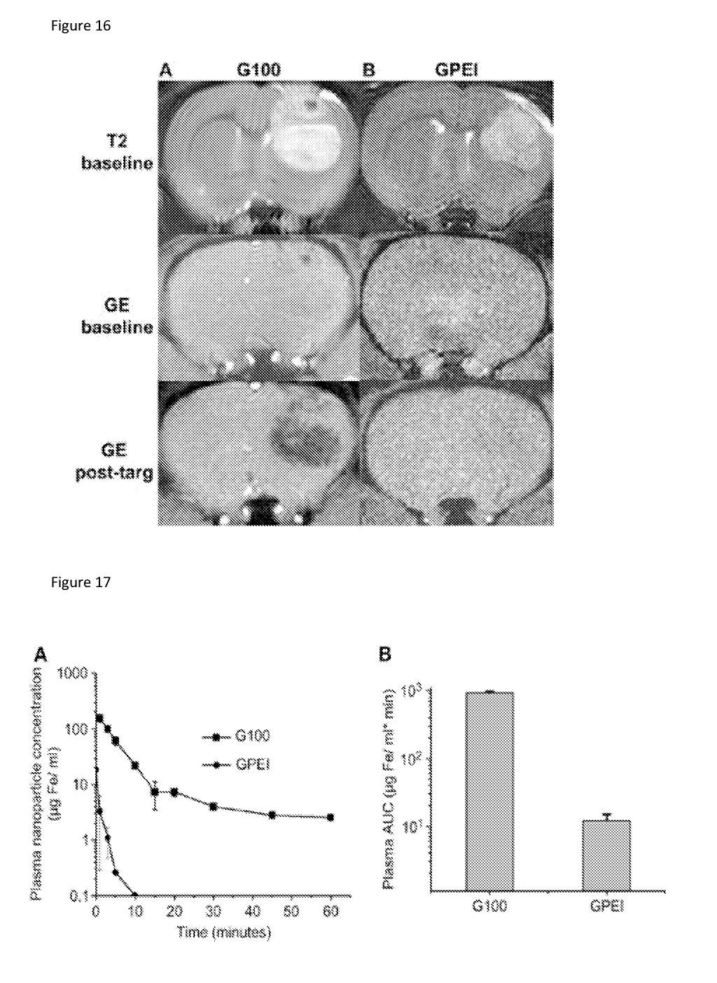

The in vivo feasibility of delivering GPEI to tumors of 9L-glioma bearing rats via magnetic targeting was examined. Delivery of GPEI via intravenous administration was performed. Magnetic resonance imaging (MRI) was used to validate nanoparticle delivery to the tumor tissue. The tumor lesion was visible as a hyperintense region on the T2-weighted MRI baseline scans of the brain (FIG. 16: T2 baseline). The presence of iron oxide nanoparticles at a particular spatial location is reflected by a pronounced hypointensity (negative contrast) on gradient echo (GE) MRI scans. Axial GE brain scan, obtained after magnetic targeting of G100 nanoparticles, shows a hypointense region corresponding to the tumor location (FIG. 16A). The loss of signal indicates G100 accumulation within the tumor lesion. In contrast, with GPEI administration no signal reduction is observed on the post targeting GE scan compared to the baseline (FIG. 16B). The lack of signal reduction indicates that GPEI nanoparticles were not accumulated in the tumor lesion.

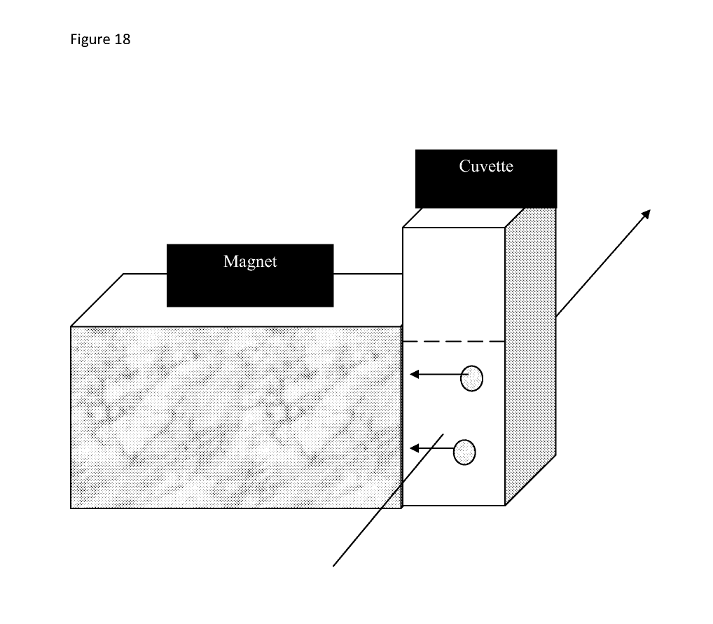

Previous work established that magnetic nanoparticles are preferably passively delivered to the brain tumor vasculature in order to be subjected to magnetic capture (Chertok et al. (2008) Biomaterials 29:487-496; herein incorporated by reference in its entirety). To assess the efficiency of GPEI passive delivery, GPEI concentrations in arterial plasma over time were determined following intravenous administration. The carotid artery ipsilateral to the tumor was used for blood collection to accurately reflect the arterial input of nanoparticles to the tumor microvasculature. FIG. 17A shows the resulting pharmacokinetic profile of GPEI compared to that of G100 after intravenous administration of both types of nanoparticles at the same dose of 12 mg Fe/kg. The positive surface charge induced substantial deterioration in nanoparticle pharmacokinetic profile, as the GPEI plasma AUC (12±3 μg Fe/ml min, FIG. 6B) was found to be 78-fold lower (p=0.002) than that of G100 (934±44 μg Fe/ml min).

The low systemic AUC of GPEI nanoparticles prompted examination of administration of GPEI via the intra-carotid route to enhance nanoparticle presentation to tumor vasculature. Passive tumor delivery advantage resulting from administration of an agent via intra-arterial versus intravenous route, Rd, can be estimated using Equation (1) supra. Assuming that the fraction of total dose lost during first passage through the tumor vasculature is small; the systemic recirculation AUC for intra-arterial administration Iᵣₐ is approximately equal to Iᵣᵥ. Based on this assumption and using literature values for the carotid blood flow and cardiac output measured in Fisher rats (see Table 3 infra), the value of R_{d} was estimated to be 27, clearly warranting intra-carotid administration.

TABLE 3
Parameters used for estimation of the advantage of intra-carotid
GPEI nanoparticle administration, R_{d}.

| Parameter | Symbol | Units | Value | Reference |
|---|---|---|---|---|
| Carotid blood flow | F | ml/min | 2.63 ± 0.14 | [29] |
| Cardiac output | CO | ml/min | 1.06 ± 5 | [30] |
| Dose | D | μgFe | 2400 | Present |
| | | | | study |
| Arterial recirculation AUC | Iᵣᵤ | μgFe/ | 12 ± 3 | Present |
| following iv injection | | ml min | | study |

FIG. 6A showed that while animals administered with PEI-MION via the intravenous route did not show any discerned difference between the post-targeting and the baseline GE scans presumably due to the extremely rapid systemic clearance of PEI-MION, animals injected with PEI-MION via carotid artery displayed a pronounced hypointense region in the post-targeting GE MRI scans corresponding to significant MION accumulation in the tumor lesion. In addition, quantitative analysis by ESR of MION accumulation in excised tumor and contra-lateral brain tissues under magnetic targeting (FIG. 6B) displayed that intra-carotid administration of PEI-MION resulted in a 30-fold increase (p=0.002) in tumor capture of PEI-MION compared to that seen with intravenous injection (222.8±66.7 versus 7.3±0.8 nmol Fe/g tissue, respectively). Also observed was a 26-fold difference (p=0.004) in targeting selectivity on brain tumor over the contralateral normal brain (222.8±66.7 versus 8.6±1.0 nmol Fe/g tissue by comparing Columns #1 & #2 from the left of FIG. 6B) following intra-carotid administration.

Delivery of Functionally Active Macromolecular Protein into the Brain Tumor

β-galactosidase (β-Gal) was selected to verify the utility of the above strategy in selectively delivering active protein into the brain tumor, due to its large size (MW: 465 kDa) and easily detectable in vivo activity. Commercial heparin-linked MION (Hep-MION) from Chemicell was used as the drug carrier. β-Gal was modified with PEI for several reasons. First, PEI was known to possess the trans-cell activity similar to that of cell-penetrating PTD peptides. Uptake by brain tumors of PEI-modified magnetic carriers via an adsorptive endocytosis mechanism has already been documented (Pulfer et al., J. Neuro-Onco1.1999; 41:99-105; Pulfer et al., J. Drug Targeting 1998; 3:215-227; each herein incorporated by reference in its entirety). Both PEI and PTD have been widely used as an effective transfection agent to enhance cellular uptake of DNA (McBain et al., J. Mater. Chem. 2007; 17:2561-2565; Park et al., J. Gene Med. 2003; 5:700-711; each herein incorporated by reference in its entirety). Furthermore, after electrostatic adsorption of the positively charged PEI-β-Gal onto the negatively charged Hep-MION, the self-assembled β-Gal/MION carries a cationic surface similar to that of PEI-MION.

Selective Delivery of PEI-β-Gal into Brain Tumors

β-Gal was chemically modified with short-chain PEI (MW: 1200 Da) using a previously established EDC activation procedure (Levin et al., Phys. Med. Biol. 1993; 38:1393-401; herein incorporated by reference in its entirety). The PEI-β-Gal conjugates thus prepared retained >80% of original enzyme activity. They were then loaded onto Hep-MION via electrostatic interaction between PEI and heparin. Results from ζ-potential measurements revealed that the surface of Hep-MION was converted from the initially negative value (−33 mV) to positive value (+24 mV) following complexation. Analysis of the protein content yielded a loading capacity of 7.2±0.4% (w/w), significantly higher than the previously reported loading of 2.8±0.16% of β-Gal on PLGA microspheres (Stivaktakis et al., J. Biomed. Mater. Res. 2005; 73:332-8; herein incorporated by reference in its entirety).

For magnetic targeting, Fisher 344 rats harboring 9L brain glioma were placed supinely on the platform with head being positioned according to the mapped magnetic field topography described above to align the tumor with the peak field density and gradient. The magnetic field density at the pole face of the magnet was adjusted to 0 T (control) or 350 mT (experimental). Animals were then injected with the PEI-β-Gal/Hep-MION complexes at a dose of 1.8 mg protein and 12 mg Fe/kg via catheterized carotid artery and retained in magnetic field for 30 min. Rats were imaged with MRI before nanoparticle administration and after the magnetic targeting as described herein. Immediately following MRI, the rats were transcardially perfused with cold PBS, dissected, and the isolated brain divided into right and left hemispheres. The tumor was carefully separated from the normal tissue of the right hemisphere, and tumor segments were stored at −80° C. for β-Gal histochemistry studies.

MRI scans showed that, in the absence of magnetic targeting, no clear difference was visually discerned between the post-targeting and the baseline GE brain scans of control animals receiving PEI-β-Gal/Hep-MION but without magnetic targeting. With magnetic targeting, the GE post-targeting scan of experimental animal again displayed a region of pronounced hypointensity, spatially corresponding to the location of the tumor lesion; validating a successful delivery of the (3-Gal-loaded MION to the tumor site.

To further assess brain distribution and functionality of the delivered protein, the excised tumor and contra-lateral brain tissues were examined for β-Gal activity. Again, no significant difference in activity (p=0.596) was observed between the tissues of control animals injected with the PEI-β-Gal/Hep-MION complexes but without magnetic targeting. In sharp contrast, tumors of magnetically-targeted rats exhibited significantly higher β-Gal activity (p<0.001) than the control tissues. In particular, a 4.7-fold higher β-Gal activity (p<0.001) was detected in tumors of the animals with magnetic targeting (636±42 μU/g tissue) than those without magnetic targeting (134±46 μU/g tissue), confirming viability of the developed magnetic targeting procedure for delivery of a functional protein to the target site. In targeted animals a 7.5-fold higher selectivity of β-Gal localization in brain tumor (636±42 μU/g tissue, p<0.001) than in the contra-lateral brain tissues (85±30 μU/g tissue) was observed.

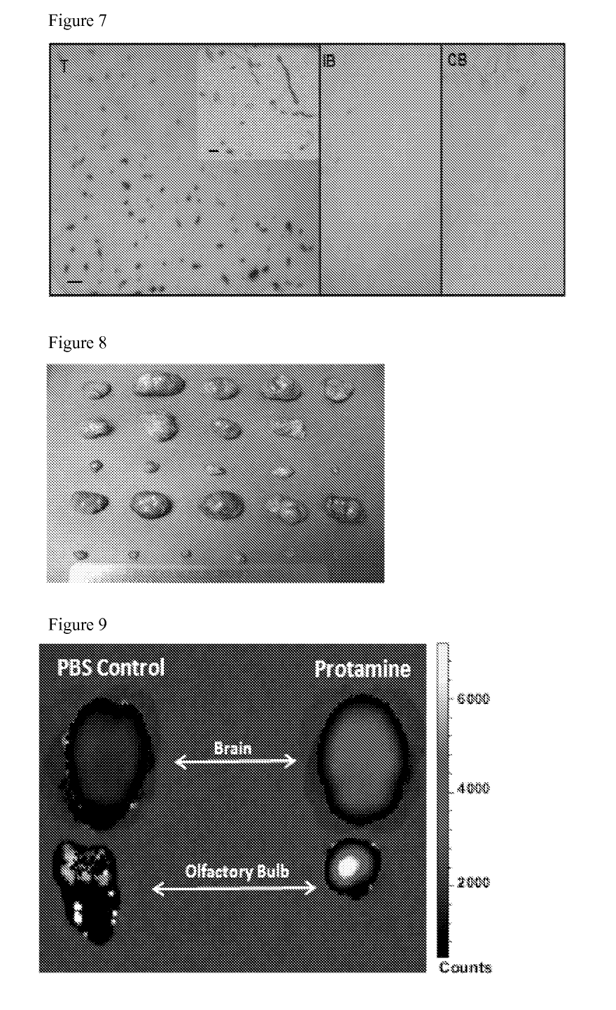

Histochemical examination of frozen brain sections of magnetically targeted rats for β-Galactivity using X-Gal staining demonstrated successful and selective delivery of β-Gal into tumor parenchyma but not normal brain regions. As shown in FIG. 7, excessive and random deposition of β-Gal in the tumor (T; Left) but not in the ipsilateral brain (IB, Middle) or contra-lateral brain (CB; Right) regions was observed, corroborating tumor-selective manner of protein delivery using magnetic targeting.

PTD-Mediated Tumor Penetration and Heparin/Protamine-Induced Regulatory Effects

Tumor regression studies on CT26 colon carcinoma-bearing BALB/c mice were carried out using TAT as the representative PTD and gelonin as the model protein drug. Gelonin is a toxin that inhibits protein synthesis by cleaving mRNA (Veenendaal et al., Proc. Natl. Acad. Sci. 2002; 99:7866-7871; herein incorporated by reference in its entirety), but is known to be unable to cross cell membranes. The TAT-gelonin conjugates (TAT-Gel) were synthesized according to a previously established procedure (Kwon et al., Expert Opin. Drug Deily. 2008; 5:1255-1266; herein incorporated by reference in its entirety). Approximately 10⁶ CT-26 tumor cells were then implanted subcutaneously into each mouse, and drug treatments were started about 3 weeks after tumor reached a size of ˜100 mm³. For convenience, tumor targeting in this pilot study was managed by direct intra-tumoral injection of the compounds. Five test compounds including: (1) PBS solution (control); (2) gelonin (100 μg); (3) TAT-Gel (100 μg gelonin equivalent); (4) TAT-Gel+heparin (20 μg); and (5) TAT-Gel+heparin (20 μg)+protamine (60 μg). Each CT-26-bearing mouse was given a total of 9 treatments (once per 2 days) and 30 days after initial treatment, mice were sacrificed and their tumors were excised and analyzed. As shown in FIG. 8, tumor growth in the control group (PBS injection) was very significant, and after 4 weeks, the average tumor mass was 3.16±0.65 g (Row #1 from top). Mice treated with gelonin alone did not display regression in tumor growth, since gelonin could not penetrate the tumor. Adding heparin to the TAT-Gel solution prior to its injection showed a complete inhibition on TAT-mediated gelonin uptake, as no statistically meaningful regression on tumor mass was observed (2.860.57 g; An average tumor weight of 2.63±0.5 g (Row #2) was observed 4 weeks after treatment. In sharp contrast, mice treated with TAT-Gel displayed significant regression in tumor growth, as the tumor weight was reduced to an insignificant value (0.33±0.12 g; RowLane #3+Row #4) when comparing with the control group (3.16+0.65 g; Row #1). Addition of protamine to the heparin-inhibited TAT-Gel, however, completely reversed this inhibition and resumed the cytotoxic activity of TAT-Gel, as tumor growth was reduced to an insignificant mass value of 0.17+0.09 g (Bottom Row).

Brain Delivery of Protamine via Nasal Administration

Since protamine binds heparin stronger than either TAT9 or LMWP90-92, it was used as a triggering agent in releasing LMWP-Drug from the Hep-MION carrier. Intra-carotid infusion of protamine has been reported to disrupt and open the BBB in various animal models (Strausbaugh et al., Brain Res. 1987; 409:221-226; Westergren et al., J. Neurochem. 1994; 62:159-165; each herein incorporated by reference in its entirety). Protamine was delivered into the brain via the non-invasive nasal route. Protamine can permeate through the nasal mucosal barrier (Zaki et al., Int. J. Pharm. 2006; 327:89-96; herein incorporated by reference in its entirety). The nasal route is the only natural reversible flow pathway involved in drainage of CSF from the brain, and is thus an ideal means to deliver drugs into the brain without encountering the BBB. The nasal route is capable of providing the highest drug bioavailability in the brain within a very short time period. For instance, mucosa-permeable drugs (e.g. anesthetics) given nasally show 2-3 fold higher concentrations in CSF than in plasma within minutes after administration (Chou et al., Int. J. Pharm. 1998; 168:137-145; Chou et al., Biopharm. Drug Dispos. 1997; 18:335-346; each herein incorporated by reference in its entirety).

Studies were conducted to demonstrate the nose-to-brain protamine delivery system. FITC-labeled clupeine protamine (300 μg) was administered via the nostril (20 μL) of the experimental mice, whereas PBS buffer was given to the control animals using the same procedures. The animals were sacrificed 1 hr after nasal administration, and their olfactory bulbs and brains were removed. Fluorescence imaging was performed on these organs using a Xenogen IVIS instrument. As shown in FIG. 9, fluorescence intensity in the olfactory bulb and brain of animals with nasal protamine administration was 10- and 2-fold stronger than the respective organs in the control animals. The background fluorescence intensity in the brain was much stronger than that in the olfactory bulb in the control animals. Semi-quantitative estimation based on the specific FITC intensity on protamine showed that μg levels of protamine were inside the brain.

### Example 2

### Drug Delivery Systems

This example describes a brain DDS comprised of four primary components including: (1) a cell-penetrating PTD peptide; (2) a macromolecular anti-tumor agent; (3) a magnetic-responsive drug carrier; and (4) a binder to link drug to the carrier and a triggering agent to release drug from the carrier.

Cell-Penetrating PTD Peptide

The low molecular weight protamine (LMWP) peptide (90-92) is used as the model PTD based on several distinct advantages. First, LMWP is almost as potent as TAT in mediating cell translocation of the attached cargos (Pastan et al., Annu. Rev. Biochem. 1992; 61:331-354; herein incorporated by reference in its entirety). A second advantage is that translocation mediated by LMWP remains in the cytosol whereas by TAT ends in the nucleus (Pastan et al., supra). As known, aside from gene delivery, most of the cytotoxic functions of anti-tumor agents such as toxins and siRNA occur primarily in the cytosol rather than in the nucleus. Thirdly, unlike TAT and virtually all the other PTD, the toxicology profiles of LMWP have already been thoroughly established. Animal studies conducted have demonstrated that LMWP is neither immunogenic (i.e. the ability to induce antibody production) (Tsui et al., Thromb. Res. 2001; 101:417-420; herein incorporated by reference in its entirety) nor antigenic (i.e. the ability to be recognized by the antibodies) (Liang et al., Biochemistry (Moscow), 2003, 68:139-144; herein incorporated by reference in its entirety). In addition, different from most of the commonly used highly cationic peptides, administration of LMWP into dogs did not elicit acute hypotensive responses or other toxicity such as complement activation (Lee et al., AAPS PharmSci. 2001; 3(2); herein incorporated by reference in its entirety). A fourth advantage is that unlike other virus-derived PTD including TAT which must be chemically synthesized, LMWP can be produced in mass quantities directly from native protamine with limited processing time and costs. Lastly, since LMWP possesses only one single —NH₂ group at the N-terminal, its conjugation to a protein drug can be precisely regulated and easily controlled using the well established N-succinimidyl-3-(2-pyridyldithio) propionate (SPDP) activation and thiolation method (Kwon et al., supra; Pastan et al., Annu. Rev. Biochem. 1992; 61:331-354 12,134; herein incorporated by reference in its entirety).

Macromolecular Anti-Tumor Agents (PE38 and ATF5-siRNA

Pseudomonas exotoxin A (PE) and the activating transcription factor 5 (ATF5)-siRNA are selected as the two model testing drugs. The two represent a protein toxin and a nucleic acid-based anti-tumor agent; two types of most widely investigated macromolecular drugs to-date. Inclusion of these two types of drugs would therefore cover a broad spectrum of large therapeutics. Although both are potent anti-tumor agents, their therapeutic outcomes could be quite different. The PE toxin is an extremely potent but non-selective toxin, and its use is therefore always accompanied with severe toxic effects (Pastan et al., supra, Chou et al., Biopharm. Drug Dispos. 1997; 18:335-346). ATF5-siRNA is a selective and safe agent against glioblastoma multiforme (GBM) (Angelastro et al., Oncogene 2006; 25:907-916, Greene et al., J. Neurochem. 2009; 108:11-22; herein incorporated by reference in its entirety).

PE is a single-chain toxin secreted by Pseudomonas aeruginosa. It is an extremely potent toxin with an IC₅₀ of less than 20 pmol, and kills cells by catalyzing the irreversible ADP-ribosylation and subsequent inactivation of elongation factor 2 (EF-2) (Pastan et al, supra). It consists of four major domains termed Ia (amino acids 1-252), II (aa 253-364), Ib (aa 365-399), and III (aa 400-613). The reaction mechanism of PE involves binding to a cell receptor via Domain Ia, and the complex is then internalized via coated pits into the endocytic vesicles where the acidic environment causes toxin unfolded. Proteolytic cleavage then occurs between residues 279 and 280, and the disulfide bond between residues 265 and 287 is also reduced, resulting in the generation of a 28-kDa N-terminal fragment and a 37-kDa C-terminal fragment. The 37-kDa fragment is transported to the Golgi apparatus and ultimately reaches the endoplasmic reticulum via a shuttle vesicle. Translocation of PE38 back to the cytosol then proceeds in a reverse direction, and EF-2 inactivation by the cytosol-transported PE38 begins.

A recombinant mutant of the 37-kDa fragment, termed PE38 (Debinski et al., J. Biol. Chem. 1995; 270:16775-8; Oshima et al., J. Biol. Chem. 2001; 276:15185-91; each herein incorporated by reference in its entirety), is selected for studies based on several benefits. First, the system relies on the unmatched trans-membrane potency of PTD to achieve cellular uptake of the toxin, and thus domains involved in cell translocation (Ia, Ib & II) become unnecessary. Indeed, depletion of Domain Ia & II that are involved in endocytosis would abort the non-selective toxicity of PE towards normal tissues, since PE receptors are known to present on almost all normal cells (Pastan et al., Annu. Rev. Biochem. 1992; 61:331-354; herein incorporated by reference in its entirety). Secondly, PE38 possesses a free —SH group on residue 287 (due to cleavage of its disulfide bond with residue 265), which can be readily linked to LMWP via the SPDP activation method (Carlsson et al., Biochem. J. 1978; 173:723-737; herein incorporated by reference in its entirety). Thirdly, PE is noted to accumulate predominantly in the liver, and because of the non-specific receptor-mediated endocytosis, it always cause significant damage to the liver. In the system described herein, however, cellular uptake of the toxin mediated by LMWP is, inhibited completely by binding with heparin.

The transcription factor ATF5 has recently emerged as a critical regulator of neuroprogenitor cell proliferation and neural tumor cell survival (Angelastro et al., Oncogene 2006; 25:907-916; herein incorporated by reference in its entirety). ATF5 belongs to the family of the basic leucine zipper proteins, with unique function to promote neuroprogenitor cell expansion and suppress their differentiation into neurons or glia (Greene et al., J. Neurochem. 2009; 108:11-22; herein incorporated by reference in its entirety). Angelastro and co-workers recently discovered that ATF5 was expressed in all 29 human glioblastomas and 8 human and rat glioma cell lines, but was not detectably expressed by mature brain neurons and astrocytes (Angelastro et al., supra; Liang et al., Biochemistry (Moscow), 2003, 68:139-144; herein incorporated by reference in its entirety). In vitro studies showed that interference with ATF5 function or expression by ATF5-siRNA caused remarkable apoptotic cell death in all glioma cell lines examined, whereas such manipulations did not affect survival of ATF5-expressing cultured astrocytes or of several other cell types that expressed this protein. Consistently, animal study using a rat glioma model displayed that intratumoral injection of ATF5-siRNA evoked aposptosis of the infected tumor cells but not of normal astrocytes and neurons outside the tumor (Greene et al., supra). These findings indicate that ATF5 is a target for treatment of glioblstomas and other neural neoplasias.

MION is used as the drug carrier. Heparin is used as the binder to hold the LMWP-Drug conjugates onto the MION carrier, and protamine is used as the triggering agent to release LMWP-Drug from MION. Both heparin and protamine are widely employed FDA-approved clinical drugs, and their interaction has been extensively documented under clinical settings and various animal models.

The prelude to heparin-induced anticoagulant functions is known to be via the binding with antithrombin III (ATIII). This means that ATIII possesses a stronger affinity to heparin than all of the other possible heparin-binding proteins in the blood. It is preferred that binding of the LMWP-modified drug to heparin is stronger than that of ATIII but weaker than that of protamine. This allows the LMWP-Drug conjugate to remain attached with its Hep-MION counterpart after intra-arterial administration, preventing it from the PTD-mediated cell internalization. The strength of this binding with heparin, however, should be weaker than protamine to allow the LMWP-Drug conjugates to be released from heparin inhibition upon protamine administration, thereby restoring the PTD-mediated trans-cell activity of the conjugate. By using a protamine sensor (Yun et al., Anal. Chem. 1995; 224:212-220; herein incorporated by reference in its entirety) as a probe to monitor the titration end-points (Yun et al., Electroanalysis 1993; 5:719-724; herein incorporated by reference in its entirety), it was possible to determine the binding strength of several proteins and PTD-like peptides (e.g. TAT, poly(Arg)7) with heparin (Yun et al., Electroanalysis 1993; 5:719-724; herein incorporated by reference in its entirety). The binding constant for LMWP (Keq=0.76×107 M-1), which is used as the PTD, sits right in-between the binding constants for ATIII (Keq=0.56×107M-1) and protamine (Keq=24.6×107 M-1)142.

Synthesis and Characterization of System Components

The cell-penetrating LMWP peptide is prepared by enzymatic digestion of native protamine with thermolysin and purified using a heparin affinity column, according to known procedures (Lee et al., AAPS PharmSci. 2001; 3(2); Schwarze et al., Trends Pharmacol. 2000; 21:45-48; Becker-Hapak et al., Methods 2001; 24:247-256; each herein incorporated by reference in its entirety). Recombinant PE38 toxin with three lysine residues in domain III at positions 590, 606, and 613 (LysPE38) in facilitating possible chemical conjugation with LMWP is commercially available from Sigma, and it can also be self-prepared using a previously established protocol (Chaudhary et al., Proc. Natl. Acad. Sci. 1988; 85:2939-2943; herein incorporated by reference in its entirety). Very briefly, plasmid pJB1PE38 encoding LysPE38 is propagated in the HB101 strain of E. coli and expressed in strain BL21 (λDE3) (Zaki et al. Int. J. Pharm. 2006; 327:89-96; herein incorporated by reference in its entirety). FITC-labeled PE38 is obtained by using the SPDP coupling method (Carlsson et al. Biochem. J. 1978; 173:723-737; herein incorporated by reference in its entirety). The sense strand sequence of ATF5-siRNA duplex is designed by using human (Accession No. NM-012068) and rat (No. NM-172336) ATF5-mRNA of: human 5′-AAG UCG GCG GCU CUG AGG UAC-dTdT-3′ (SEQ ID NO:1), rat 5′-AAG UCA GCU GCU CUC AGG UAC-dTdT-3′ (SEQ ID NO:2), respectively; according to the established characteristics of siRNA targeting constructs 7. The RNA sequence: 5′-GCG CGC UUU GUA GGA UUC G-dTdT-3′ (SEQ ID NO:3) is used as a negative control for siRNA activity. The ATF5 siRNAs for human and rat are modified with a cysteine residue at the 5′-end of the RNA strand for future conjugation with LMWP. In addition, FITC- or rhodamine-labeled siRNA (at 3′-end of the complementary strand) are also prepared.

LMWP-Drug Conjugates

Both LMWP-PE38 and LMWP-ATF5-siRNA are synthesized. Since LMWP contains only a single —NH₂ group at the N-terminal whereas PE38 possesses a free —SH group on residue 287, synthesis of 1:1 (molar ratio) LMWP-PE38 conjugate is performed utilizing a modified SPDP coupling method (Steiniger et al., Int. J. Cancer 2004; 109:759-767; herein incorporated by reference in its entirety). Briefly, the —NH₂ end of LMWP is activated by reacting with 5-fold molar excess of SPDP for 2 hrs at room temperature. After removal of the excess SPDP using a heparin affinity column, the SPDP-activated LMWP is reduced by adding 50 mM dithiothreitol (DTT) to produce LMWP with a reactive —SH group at the N-end. PE38 is then linked to LMWP-SH via its free —SH group to yield LMWP-PE38 conjugate with a cytosol-cleavable S—S bond. Identification and characterization of the synthesized LMWP-PE38 conjugates is carried out by using standard MALDI-MS and SDS-PAGE analytical methods. Biological activity of LMWP-PE38 is analyzed by the ADP ribosylation assay (with wheat germ extract serving as the source of EF-2), according to the protocol by Collier and Kandel (J. Biol. Chem. 1971; 246:1496-1503; herein incorporated by reference in its entirety).

Unlike PE38, covalent conjugation of LMWP to ATF5-siRNA could result in cationic LMWP and anionic siRNA self-assembling into insoluble ionic complexes. Although results (Park et al., J. Gene Med. 2003; 5:700-711; Chiu et al., Chem. Biol. 2004; 11:1165-1175; Turner et al., Blood Cells Mol. Dis. 2007; 38:1-7; each herein incorporated by reference in its entirety) indicated that non-covalent packaging of siRNA with the cell-penetrating peptides (e.g. TAT) would still yield a certain degree of PTD-mediated cellular internalization of siRNA, it was generally acknowledged that the most effective means for enhancing the cellular uptake is through covalent linking of a siRNA duplex to a PTD prior to cellular treatment (Meade and Dowdy, Adv. Drug Del. Rev. 2007; 59:134-140; herein incorporated by reference in its entirety). The present example uses a strategy that is derived from the movement of the PEG polymer and its subsequently yielded shielding effect to override the aforementioned obstacles. In general, the strategy calls for linking LMWP first with PEG (MW: ˜5 KDa; (the MW of the PEG molecule should exceed that of LMWP or siRNA) containing heterobifunctional reactive groups of NHS (N-hydroxysuccinimide) and maleimide at the two terminus. Since LMWP possesses no —SH group but only one —NH₂ group at the N-end, it can only be linked to the NHS terminus of the PEG polymer. Cysteine is then added to the LMWP-PEG-maleimide conjugate to convert the maleimide end to contain a —NH₂ group via the formation of a thioester bond between the —SH group on cysteine and maleimide group on LMWP-PEG. The LMWP-PEG-NH₂ product is then activated again with SPDP and thiolated with dithiothreitol according to the previously described procedure (Park et al., J. Controlled Release 2002; 78:67-7; herein incorporated by reference in its entirety). The reactive LMWP-PEG-SH thus prepared can then be linked to ATF5-siRNA, which is synthesized to possess a terminal cycteine residue, through a cytosol-cleavable disulfide linkage. Since both reacting compounds contain only a single reactive —SH group, formation of 1:1 (siRNA:LMWP) covalently linked conjugates is therefore warranted.

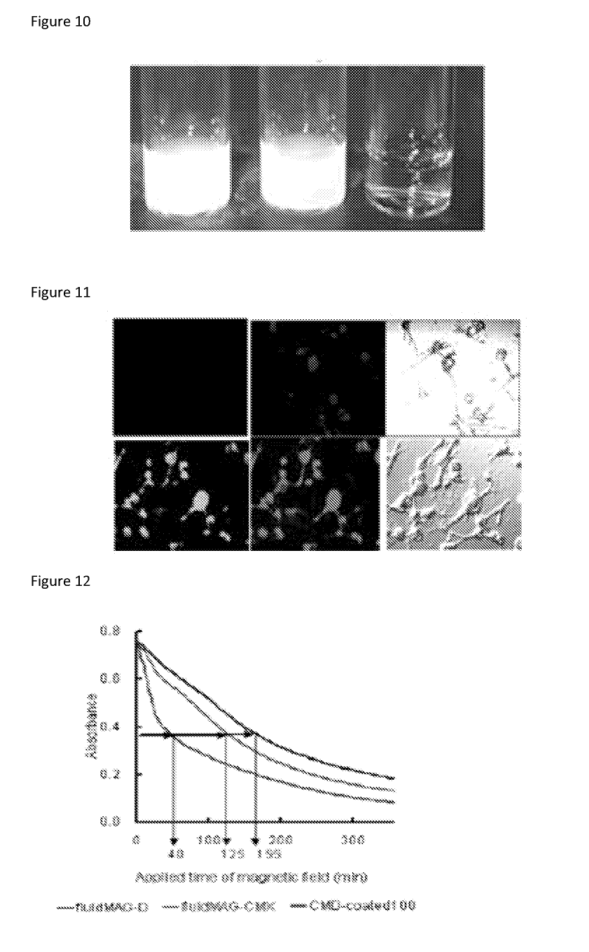

Studies utilizing a representative anti-HIF1α anti-sense oligodeoxynucleotide (AS-ODN) revealed that the PEG chain on LMWP-PEG could shield LMWP from charge-induced complexation with AS-ODN during the coupling process thereby producing a transparent reaction mixture (FIG. 10; Right); unlike direct mixing of ODN with LMWP (Left) or SPDP-activated LMWP (Middle) in which significant precipitation of the insoluble charged complexes was observed. The dynamic spatial movement of PEG prevented the formation of intra-molecular hairpin structure between the anionic ODN and cationic LMWP in the conjugate, as FIG. 11 (Bottom) displayed a successful cellular uptake of the LMWP-ODN (labeled with FITC) conjugate in vitro. ODN without the attachment of LMWP failed to internalize the cells (FIG. 11; Top). A relatively high yield (>60%) of the soluble covalently linked LMWP-ODN conjugate was recovered from the reaction mixture, after incorporating the solution with citrate (3-5% in total concentration) to assuage charge-charge interaction. Characterization of the synthesized LMWP-ATF5-siRNA conjugates is carried out by using the standard MALDI-MS and SDS-PAGE analyticalmethods.

Heparin-Coated, Lame-Core MION (Hep-MION) with Superparamagnetic Behavior

Large-core Hep-MION is synthesized with uniform morphology and desired size (100-200 nm), while retaining a large core structure yielding superior magnetophoretic mobility and exhibiting superparamagnetic behavior for both MRI and other clinical applications. In this method, monocrystalline, ultrafine (10 nm) iron oxide nanoparticles possessing superparamagnetic behavior is first be prepared. These nanoparticles are then be treated with citrate or glycine to produce negatively or positively charged surfaces, respectively. These charged nanoparticles remain under the size threshold essential for retaining superparamagentic behavior without forming tightly bound aggregates, because the strong inter-particle magnetic attraction is offset by the charge-induced repulsive force. Self-assembly of these −/+ charged nanoparticles then takes place via electrostatic interaction to yield a loosely agglomerated core of magnate possessing both the superparamagnetic behavior and high magnetophoretic mobility. The large magnetic cores thus prepared are stabilized by coating with a biocompatible dextran polymer containing immobilized heparin (referred as Hep-D). A loading of 80 nmol heparin/mg MION was attained.

Carboxymethyl dextran-coated MION comprising a large magnetic core and desirable hydrodynamic size of −100 nm (termed CMD-coated 100) was prepared by utilizing the above approach. Compared with commercial dextran-coated MION (fluidMAG-D) acquired from Chemicell, both samples displayed the presence of superparamagnetic behavior under the influence of an external magnetic field; as SQUID data showed that neither of the demagnetization curves displayed a visible hysteresis loop (i.e. remnant magnetization). FIG. 12 summarizes the magnetophoretic mobility results of CMD-coated100 and two commercial Chemicell MION products, fluidMAG-D and fluidMAG-CMX (coated with carboxymethyl dextran), of which the magnetophoretic mobility was measured via a direct-viewing technique by placing MION suspension in a cuvette surrounded by a magnet and then measuring the migration of nanoparticles by disappearance of the light scattering using a spectrophotometer. As seen, under an external magnetic field, ther large-core CMD-coated100 product moved much quicker than either of these two commercial products. By using the migration half-time (t1/2), defined as the time required for the absorbance in the cuvette to drop by 50%, as the measuring index (i.e. the shorter the t1/2, the quicker the particle moves), CMD-coated100 displayed a t1/2 of 40 min (bottom curve), nearly 3 to 4-fold faster mobility than fluidMAG-CMX (125 min; middle) and fluidMAG-D (155 min; top).

Characterization of the large-core Hep-MION is carried out using a standard protocol (Debinski et al., J Biol. Chem. 1995; 270:16775-80; herein incorporated by reference in its entirety). Briefly, morphology is examined by transmission electron microscopy (TEM) and particle size is measured by light scattering using the NICOM software. Content of the heparin-dextran coating is determined by using a Q50 thermogravimetric analyzer (TGA) under N₂ atmosphere with a temperature increase at a rate of 20° C./min. Superparamagnetic properties is assessed by using a superconducting quantum interference device (SQUID) (Quantum Design, USA). Iron content is assayed by using an Optima 2000 DV inductively coupled plasma-optical emission spectroscopy (ICP-OES).

Because of the very rapid clearance of MION (t_{1/2}˜5-10 min), the optimal opportunity to achieve the highest nanoparticle accumulation at the tumor lesion is to capture MION right after their intra-arterial administration and during their first passage into the circulation. Therefore, the higher magnetophoretic mobility the larger degree of MION capture by the tumor via aid of the magnetic force.

In Vitro Evaluation

In vitro characterization of both conjugates is carried out in cell culture medium using various assays. Thymidine incorporation (i.e. protein synthesis inhibition) assays are performed by using a slightly modified protocol of Oshima and Puri (J Biol. Chem. 2001; 276:15185-91; herein incorporated by reference in its entirety). Briefly, cells (10⁴ cells/well) are grown in culture dishes and incubated with various concentrations of LMWP-PE38 for 52 hrs at 37° C. and pulsed with [methyl-³H]thymidine (0.5 μCi/mL) for an additional 12 hrs. The LMWP-WT-PE38 conjugate and LMWP alone are used as the controls. At the end of the pulse, the medium is aspirated, and cells are rinsed twice with ice-cold PBS. The rinsed cells are then fixed with 5% ice-cold trichloracetic acid overnight at 4° C. Incorporation of [³H]thymidine is detected by scintillation counting and expressed as disintegration per min/10³ cells. A second set of non-fixed cells is used as an internal control to estimate cell numbers.

ADP ribosylation activity is measured by using the method of Bachran et al. (Clin. Chem. 2007; 53:1676-1683; herein incorporated by reference in its entirety). Endogenous EF-2 from yeast is purified and used for this assay. Biotinylated NAD+ and LMWP-PE38 are mixed in 50 mM Tris buffer (pH 7.6) containing 1 mM EDTA and dithiothreitol for 1 h at 37° C. Samples are then subjected to SDS-PAGE and immunoblotting assay using goat anti-biotin antibody according to procedures described previously (Kaup et al., J. Biol. Chem. 2002; 277:38494-502; herein incorporated by reference in its entirety). For negative controls, heat-denatured PE38 is used.

Cytotoxicity of the LMWP-PE38 conjugates is carried out against C6 rat glioma cell line using a previously established MTT assay (Green et al., Cell 1988; 55:1179-1188; herein incorporated by reference in its entirety). Cells (1×10⁴ cells/well) grown with 75% confluency are incubated with various concentrations of LMWP-PE38. Cell proliferation is measured over 48 hr, during which culture medium is removed and replaced with PBS containing 2 mg/mL MTT. The absorbance is measured at 570 nm after addition of DMSO, and the survival ratio is determined from the absorbance ratio between treated and un-treated cells. The LMWP-WT-PE38 conjugate is used as a control.

Transfection with ATF5-siRNA (80 μmol/well) and pCMS-EGFP vector (1 μg/well) by using LipofectAMINE 2000 (0.15%, v/v) is carried out according to the manufacturer provided protocol. Fresh medium is added 9 hr after transfection, and experiments are performed 48 hr after transfection. For the control, cells are transfected with pCMS-EGFP vector alone. Equivalent concentrations of the LMWP-ATF5-siRNA conjugates are used to transfect the cells.

Transfected cells are harvested by scraping in RIPA lysis buffer (Amersham Biosciences), followed by centrifugation for 15 min at 4° C. Cell proteins are resolved by SDS-PAGE, transferred onto nitrocellulose membrane, blocked for 1 hr in PBS containing 5% milk and 1% BSA, and then immunolabeled overnight using ATF5 (1:1000) and Actin (1:3000) antiserum in PBS containing 5% milk. For ATF5 and Actin protein detection, the blots are probed with anti-rabbit and anti-goat horseradish peroxidase (HRP)-conjugated antibody (Santa Cruz, Calif.), respectively, and then visualized on film using Amersham's enhanced chemiluminescence (ECL) detection kit.

Cells are seeded in 1.5 mL culture medium at a density of 1×10⁶ cells/well. Thereafter, cells are washed twice with 1 mL cold PBS and fixed by drop-wise addition of 1 mL of chilled (−20° C.) 70% ethanol with gentle vortexing. After incubation for 24 h at 4° C., cells are resuspended in 100 μL PBS, followed by addition of 2 μL of RNase and 10 μL of propidium iodide. Following incubation for 1 hr at room temperature, cell apoptosis is measured using flow cytometry.

BrdU incorporation assay is conducted using a modified procedure of Mason et al. (Mason et al., Mol. Cell. Neurosci. 2005; 29:372-380; herein incorporated by reference in its entirety). Briefly, BrdU is added to the RG2 cell cultures after transfection to label the S-phase of cell cycle. These cells are then fixed with 4% PFA for 5 min at 4° C., incubated with 20% NGS (normal goat serum) for 30 min and then with a mouse monoclonal anti-BrdU antibody (Boehringer Mannheim) overnight at 4° C. After incubation with anti-rabbit HRP-conjugated antibody, BrdU-stained cells are examined using an Olympus FV-500 confocal microscope.

The RG2 cells are fixed with 4% paraformaldehyde, incubated with 20% normal goat serum (NGS)/PBS for 30 min to block non-specific binding, and then incubated with a rabbit polyclonal anti-ATF5 antibody (1:200) in 20% NGS/PBS overnight at 4° C. After incubation with FITC-linked secondary antibodies (1:100; Southern Biotechnology) in 20% NGS/PBS for 45 min at 25° C., cells are counter-stained with DAPI (1:10,000) and examined using a confocal microscope.

Cytotoxicity of the LMWP-ATF5-siRNA conjugates is carried out against C6 rat glioma cell line using the same MTT assay. Scrambled LMWP-siRNA is used as the control.

Release of the drug from the carrier by ionic desorption is examined in physiological buffer. Standard time course studies of the drug release kinetics are conducted by dispersing the Drug/MION complexes in PBS, centrifuging the aliquots of samples withdrawn at various time points, and then measuring the absorbance (260 and 280 nm for ATF5-siRNa and PE38, respectively) in the supernatant.

### Example 3

### In Vivo Evaluation of the Efficacy/Pharmacokinetics/Biodistribution/Toxicity of the System

The heparin-coated MION contains a heparin loading of 80 nmol heparin per mg of MION dry weight. Each milligram of MION by dry weight gives a Fe content of about 0.5 mg. The MION dose used in animal studies is about 4.8 mg Fe per rat (i.e. 9.6 mg MION dry weight/rat), which is also the dose being routinely used by other investigators for MRI or targeting without any safety concern (Harisinghani et al., AJR 1999; 172:1347-1351; Shen et al., Magn. Reson. Med. 1993; 29:599-604; Weissleder et al., AJR 1989; 152:167-173; each herein incorporated by reference in its entirety). Using the literature reported binding stoichiometry of 2.5 between heparin and LMWP136, this MION dose is capable of carrying approximately 1.92 μmol of the drug per rat (i.e. 80 nmol heparin/mg MION×9.6 mg MION/rat×2.5 MWP/heparin). A brain tumor accumulation of ˜0.3% of the total administered MION dose per gram of the tissue was obtained (See Above). Thus, approximately 576 μmol drug per tumor (i.e. 1.92 μmmol drug/rat×0.3%/g tissue×100 mg tumor weight) is accumulated.

The IC₅₀ of PE38 is reported to be <20 μmol (Husain et al. J. Neurooncol. 2003; 65:37-48; herein incorporated by reference in its entirety); ˜4-5 orders of magnitude lower than the 576 nmol of drug that is delivered to the tumor lesion. Since the PE38 dose is 50 μg/kg (i.e. 10 μg PE38 per rat for a 20 by following a well-established testing protocol (Husain et al., J Neuro-Oncol. 2003, 65, 37-48; herein incorporated by reference in its entirety), PE38-loaded MION complexes are diluted with sham MION complexes (e.g., Hep-MION without the drug)

Since there has been no literature report of ATF5-siRNA delivery, the required effective dose of ATF5-siRNA in silencing a brain tumor is estimated based on the dose of VEGF-siRNA that has been attached to MION (Medarova et al., Nature Medicine 2007; 13:372-377; herein incorporated by reference in its entirety) and used for silencing tumor in vivo (Takei et al., Cancer Res. 2004; 64:3365-3370; herein incorporated by reference in its entirety). Information gathered from these references indicated that delivery of 500 μmol VEGF-siRNA within the tumor lesion was able to completely suppress tumor growth in PC-3 mouse. According to the above assessment (576 nmol of drug per rat tumor), at least this dose threshold is used. MION carriers containing a total ATF5-siRNA dose of ˜2.0 μmmol siRNA/rat are employed in the rat study.

A detailed pharmacokinetic (PK) study is used to estimate the required nasal protamine dose in order to reach the required dose threshold in the brain for successful triggering action. To conduct the PK study, the —NH₂ group at the N-terminus of protamine is linked with FITC using the previously described SPDP method (Park et al., J. Controlled Release 2002; 78:67-79; herein incorporated by reference in its entirety). Solution containing FITC-protamine (25 μg/μL) are then intranasally administered to the anesthetized rats (20 μL/nostril) through a polyethylene tubing attached to a Hamilton microliter syringe. At different time points (15 min, 30 min, 1 hr, 2 hr, 4 hr and 6 hr), blood samples are collected by cardiac puncture. The animals are then perfused transcardially with 350 mL of saline buffer, followed by collection of brain tissues. The olfactory bulb, cerebellum and cerebrum are dissected and homogenized, and the supernatants are collected. Protamine concentrations in the brain compartments as well as plasma are determined by measuring the FITC intensity using a fluorescence spectrometer. Six rats are used for each time point.

Intracerebral 9L tumors are induced in male Fisher 344 rats (˜150 g) using a previously established protocol (Chertok et al., Biomaterials 2008; 29(4):487-496; herein incorporated by reference in its entirety). Briefly, cell suspension (10⁶ cells) are implanted in the right forebrain of the animals at a depth of 3 mm beneath the skull through a 1-mm-diameter burr hole filled with bone wax to prevent extracerebral tumor extension. Animals are imaged with MRI every other day beginning at day 7 after cell implantation to monitor tumor volumes. At least 4 image data sets are collected prior to initiation of treatment to determine the baseline kinetics of tumor growth in each animal. Once the tumor volume reaches 60-80 μL, the right carotid artery of glioma-bearing animals is catheterized as described above. Briefly, the right carotid artery of anaesthetized animals is exposed by blunt dissection. The catheter composed of silica and PE-10 tubing is inserted cephalad through the arterial wall. The tiny incision is rapidly resealed with a drop of tissue adhesive (3M Vetbond) to maintain intact blood flow through the catheterized artery.

Animals are anesthetized with 1.5% isoflurane/air mixture and imaged on a 18-cm horizontal-bore, 7 Tesla Varian (Palo Alto, Calif.) Unity Inova imaging system using a 35-mm-diameter quadrature RF head coil (USA Instruments Inc, OH). Diffusion MRI is performed according to a previously described procedure 156. Briefly, an isotropic diffusion-weighted sequence with two interleaved b-factors (Δb=1148 s/mm²) is employed and the following acquisition parameters are used: repetition time (TR)=3500 ms, echo time (TE)=60 ms, field of view=30×30 over 128×128 matrix, slice thickness=1 mm, slice separation=0.2 mm. Thirteen axial slices are acquired to provide a contiguous image data set of the rat brain. The images are T2-weighted to allow tumor volume determination as described by Kim et al. (Clin. Cancer Res. 1995; 1:643-50; herein incorporated by reference in its entirety), and estimation of the tumor coordinates within the animal head for alignment within the magnetic field. To determine nanoparticle distribution in the brain, thirteen gradient echo (GE) axial slices of the brain are collected before MION administration (baseline scans) and immediately following magnetic targeting. GE images are acquired with the following parameters: TR=20 ms, TE=5 ms, field of view=30×30 over 128×128 matrix, slice thickness=1 mm, slice separation=0.2 mm.

The configuration of the magnetic setup and the animal positioning with respect to the magnet is optimized to direct the highest magnetic force towards the targeted tumor region. The magnetic setup consists of a small cylindrical ferromagnet mounted on the tapered pole of the standard dipole electromagnet (Model 3470; GMW associates, San Carlos, CA). Magnetic field simulations using Maxwell 3D magnetic modeling software (Ansys inc., Canonsburg, Pa.) predicted that the ferromagnet thus positioned would deflect the magnetic flux and generate a peak of magnetic field density and gradient on its pole face. To experimentally determine spatial coordinates of this peak, magnetic field density is measured using a teslameter equipped with a 3-D Hall sensor. The corresponding topographic maps of magnetic field density is plotted using MathCadll software package (Mathsoft, Mass.). For animal alignment within magnetic field, longitudinal and lateral location of the tumor lesion relative to the middle of the eye and the midline of the head, respectively, are calculated from the low b-factor diffusion-weighted MRI brain scans. Briefly, the rat is placed supinely on the platform and its head, marked with MRI-derived coordinates of the glioma lesion, and positioned according to the mapped magnetic field topography to align the tumor with the peak field density and gradient. The magnetic field density at the pole face of the ferromagnet is adjusted to 350 mT.

In general, glioma-carrying rats implanted with carotid catheters are distributed into 3 groups with treatment of: Group #1: PBS solution (control); Group #2: LMWP-Drug/Hep-MION complex+intranasal PBS (2nd control; also for testing release of the LMWP-Drug conjugate due to natural ionic desorption; and Group #3: LMWP-Drug/Hep-MION complex+intranasal protamine (for testing protamine-triggered release of LMWP-Drug). When ATF5-siRNA is used as the drug, a 4th testing group (Group #4) involving treatment of LMWP-siRNA(scrambled)/Hep-MION complexes+intranasal protamine is added. On the day of treatment, animals are pre-scanned with diffusion-weighted MRI and GE MRI. Diffusion-weighted images are used for calculating the pre-treatment ADC maps and estimating the tumor coordinates within the animal head. Alternatively, the GE MRI pre-scans are used to visualize post-treatment distribution of MION within the brain. For magnetic targeting, the animals are aligned in the magnetic field. The rats are then injected with the: LMWP-Drug/Hep-MION complexes at a dose of 48 mg MION/kg body weight (equivalent to an iron content of 24 mg Fe/kg) via catheterized carotid artery and retained in magnetic field for 30 min (for the control experiments an equivalent volume of PBS solution are injected). This MION dose has been demonstrated in the literature to be well tolerated in animals without any reported toxic effect (Harisinghani et al., AJR 1999; 172:1347-1351; Shen et al., Magn. Reson. Med. 1993; 29:599-604; Weissleder et al., AJR 1989; 152:167-173; each herein incorporated by reference in its entirety). Each administration of the Drug/MION complex contains a total dose of 10 μg/rat or 2 μmmol/rat for PE38 and ATF5-siRNA, respectively. To validate MION delivery to the tumor site, the GE MRI scans of the rat brains are re-acquired immediately following magnetic targeting. Hypointense region on GE scans, discernable from the pre-scan and co-localized with the tumor location signify MION delivery to the tumor site. Once MION accumulation within the targeted tumor lesions is confirmed, the rats are intranasally administered with protamine (20 μL) through the right nostril as previously described.

The study is carried out in four phases. In Phase I, a single dose treatment is assessed. The goal is to assess the ability of the proposed treatment to interfere with the tumor growth in vivo. Phase II has three purposes: (1) to establish the dose-response profile in order to select the lowest dose which results in maximal tumor regression; (2) to evaluate response kinetics to a single dose treatment in order to estimate dosing frequency; and (3) to estimate the total number of doses required to reduce the tumor mass to an insignificant value. In Phase III, multi-dose treatment regimens are explored. The goal of this phase is to validate the dosage regimen model designed in Phase II regarding to tumor volume reduction. Finally, in Phase IV, the therapeutic efficacy of a multi-dose treatment regimen is examined.

In Phase I, 10 rats are distributed to each of the 3 (PE38 as the testing drug) or 4 (ATF5-siRNA as the drug) animal groups. The response of the neoplasm to therapy is evaluated by monitoring changes in water diffusion and tumor growth. Diffusion MRI images of the rat brain are collected every 2 days. Since changes in tumor volume might not become apparent for up to two week following treatment (Stegman et al., Gene Ther. 2000; 7:1005-10; herein incorporated by reference in its entirety), the image collection is continued for up to three weeks after treatment. As noted, alterations in tissue water diffusion were shown to provide a surrogate marker for an early detection of therapeutic response preceding macroscopic changes in tumor volume and growth rate (Stegman et al., supra; Hall et al., Clin. Cancer Res. 2004; 10:7852-9; herein incorporated by reference in its entirety). Treatment-induced damage to integrity of cell membranes is expected to increase the mobility of water in the affected tissue. Using diffusion MRI, the in vivo diffusivity of water can be accurately quantified in terms of apparent diffusion coefficient (ADC). The post-treatment increase in ADC values provides an early indication of therapeutic effectiveness. The ADC values for the test and control groups are estimated at each time point and compared to evaluate the tumor damage of the test treatment versus controls. For the two drugs, a total of 70 rats (30 for PE38 and 40 for siRNA) are included in Phase I.

In Phase II, the dose-response profile is studied and kinetics of response to a single dose evaluated. Aside from Group I, all the other groups are tested with 4 different dose regimens. For PE38, the 4 doses are 5, 10, 25, and 50 μg/rat, whereas for ATF5-siRNA the doses are 1, 2, 5, and 10 μmmol/rat. Ten rats are included in each testing group. Diffusion MRI images of the rat brain are collected every 2 days for up to three weeks after treatment. The ADC(t) values are estimated and change in ADC (ΔADC(t)) as compared to the baseline is calculated at each time point for each animal. The dose-response curve id constructed by plotting mean ΔADCd(t) values as a function of dose at time t. It is expected that the dose-response curve will exhibit sigmoidal behavior. The dose at which a sigmoid starts to level off is defined as the “target dose” for Phase III efficacy study. The ΔADC(t) of the target dose is plotted as a function of time. The time interval at which the maximal change ΔADC(t) value is achieved is defined as the “dosing interval” for the Phase III efficacy study. To also estimate the treatment-induced retardation in tumor growth and the in vivo fraction of cell kill, tumor volumes are calculated from the low b-factor diffusion-weighted images collected for the animals administered with the target dose. The tumor volume data is used to estimate the fraction of cell kills and the post-treatment tumor growth constant. A total of 220 rats (90 for PE38 and 130 for siRNA) are included in Phase II.

In Phase III, the multi-dose treatment regimen is evaluated. The purpose of this phase of study is to determine feasibility of theoretical estimation of the total number of doses required to reduce the tumor volume below the threshold value. The fraction of cell kill and post-treatment tumor growth constant parameters, estimated in single-treatment Phase II, is used to predict the tumor volume after each dose of the multi-treatment regimen using fractionated dosage method. Theoretically estimated profile of tumor growth during multi-treatment regimen is compared to experimental data to assess the model validity. For experimental data acquisition, 15 rats are included in each group and treated with the target dose determined in Phase II. Five treatment doses are given at a dosing interval also determined in Phase II. Diffusion MRI images of the rat brain are collected every 2-3 days throughout the treatment and for up to three weeks following the last administered dose. Tumor volumes are calculated from the low b-factor diffusion-weighted images as described before. Mean experimentally acquired tumor volumes at each time point are compared to the theoretically predicted volumes to validate the model. Once the model is validated, it is used to iteratively calculate the number of does required to reduce the tumor volume below the pre-defined threshold. This number of doses is used in the efficacy study of Phase IV. A total of 105 rats (45 for PE38 and 60 for siRNA) are included in Phase III.

The purpose of Phase IV is to evaluate therapeutic efficacy of the proposed treatment at the dose regimen designed in Phases II and III. Fifteen rats are included in each group and treated with the target dose determined in Phase II. The total number of treatment doses calculated in Phase III is given at a dosing interval determined in Phase II. The therapeutic efficacy is assessed by monitoring changes in water diffusion and tumor growth. Diffusion MRI images of the rat brain are collected every 2-3 days throughout the treatment and for up to three weeks following the last administered dose. ADC values and tumor growth kinetics are estimated and compared as a measure of treatment efficacy. In addition, survival time of each animal is recorded within 90 days following tumor implantation. At day 90, the study is terminated. The survival of untreated 9L-gliosarcoma harboring rats was previously reported to be 22±2 day Implantation (Kim et al., Clin. Cancer Res. 1995; 1:643-50; herein incorporated by reference in its entirety), thus 90-day observation periods are sufficient to provide treatment survival benefit compared to controls. A total of 105 rats (45 for PE38 and 60 for siRNA) are included in Phase IV.

ADC maps are calculated from the diffusion-weighted image sets using the equation (Kim et al., 1995, supra): ADC={ln(S1/S2)}/(b2−b1) (formula 2) where S1, S2 are the signal intensities on images acquired with b1, b2 diffusion gradient values, respectively. The mean ADC values and the ADC pixel value histograms is calculated on the manually outlined tumor regions of interest (ROIs) across brain slices. The pre-treatment mean ADC value (ADC(t=0)) is subtracted from the post-treatment values (ADC(t)) to evaluate the change in ADC versus baseline (AADC(t)) as a function of time: AADC (t)=ADC(t)−ADC(t=0) (formula 3), the mean post-treatment ADC values of the variance followed by Tukey's multiple comparisons test at a nominal significance level of 0.05. The ADC histograms of the tumor mass acquired over time are plotted in a stacked format to allow visualization of time-dependent tumor response to treatment (Hall et al., 2004, supra).

The volumes of the intracranial tumors are quantified as previously described (Kim et al., 1995, supra). For volume measurements at each time point, the area of tumor visualized in each slice of low b-factor diffusion-weighted images is manually outlined using MATLAB region of interest (ROI) routine. The outlined tumor area in each cross-sectional image is multiplied by the slice separation (0.2 mm) to calculate the tumor volume in each slice. The tumor volumes of individual slices are summed to yield the total volume of the tumor. Although tumors as small as 2 mm³ (about 2 mm in diameter) can be measured using MRI, tumors with a minimal volume of 8-10 mm³ are used for the initial volumetric time point to maximize accuracy of the initial tumor volume determination.

The pre-treatment volume measurements over time are used to estimate the baseline tumor growth rate in each animal prior to treatment. The tumor volume data as a function of time is fitted to the exponential growth model (157): V₍ₜ₎=V₀10^{kt}, where V₍ₜ₎ is tumor volume at time t, V₀ is the initial tumor volume and k is the tumor growth rate constant. The pretreatment tumor growth rate constant (k) is then estimated from the exponential fit. The tumor volume data acquired after the treatment is used to estimate the post-treatment growth rate constant (k′) and the fraction of cells killed by treatment (fₖ) using a model for post-treatment tumor volume.

Vₚ(t)=└(1−fₖ)V(t_{T})e^{k(t-t}^{_{T}}⁾┘+f_{K}V(t_{T}),

where Vₚ(t) is the post-treatment tumor volume measured at time t and V(t_{T}) is tumor volume at the time of treatment (t_{T}). Non-linear fitting of the post-treatment MRI volumetric data to the model allows estimation of parameters k′ and fk. The change in the tumor growth rate due to treatment is defined as Δk=k−k′ and calculated for each individual animal. The mean Δk is then be compared across the treatment groups using one-way analysis of variance followed by Tukey's multiple comparisons test at a nominal significance level of 0.05.

Post-treatment tumor volume for multi-treatment regimen is estimated by using a previously established model (Ross et al., NMR Biomed. 2003; 16:67-76; herein incorporated by reference in its entirety). In brief, post-treatment tumor volume is approximately the sum of the volume of dead cell killed by treatment and the volume of viable cells exponentially growing from the survived fraction with a growth constant k′. The post-treatment tumor volume (Vp) expressed as a function of the cell kill fraction is given by following:

Vₚ(tₜ)=fₛₘ(i)V(t_{T})exp[k′(tₜ−t_{T})]+ΣVₖ(i),

Where fₛₘ(i) is surviving fraction of cells by the ith treatment time as expressed by fₛₘ(i)=fₛ₁*fₛ₂*fₛ₃ . . . fₛ₍ᵢ₋₁₎. k′ is the post treatment rate constant, V(t1) is tumor volume at the time of initial treatment (t₁) as described by V₍ₜ₁₎=Vₒₑₓₚ₍ₖₜ₁₎ and Vₖ₍ᵢ₎ is the last volume of tumor cell killed by the i-th treatment. The last term describes the cumulative sum of dead cell volume from each treatment does as represented in:

$$\text{?} {V}_{k} ( i ) = {f}_{k} ( 1 ) V ( \text{?} ) + {f}_{k} ( 2 ) {V}_{L} ( 2 ) + {f}_{k} ( 3 ) {V}_{L} ( 3 ) + … + {f}_{k} ( i - 1 ) {V}_{L} ( i - 1 ) , \\ \text{?} \text{indicates text missing or illegible when filed}$$

where V_{L(i)} is the volume of the live cell fraction at the ith treatment time and fₖ=1−fₛ.

Survival of the four treatment groups is compared using a log-rank test for the difference in median survival time from tumor implantation at a nominal significance level of 0.05161. This analysis is followed by pair-wise log-rank tests on selected groups to determine which groups have significantly different median survival times. Kaplan Meier plots are made to visualize the survival data.

Evaluation of the toxicity of the system, particularly related to liver, is performed. As a key organ of the RES, the liver is a major depository for MION soon after administration. To assess toxicity of the system, blood, plasma and urine samples are collected during the course of the animal studies (at time before the study begins and on the last day). Levels of hematocrit, hemoglobin, RBC, WBC, and platelet counts as well as of albumin, globulin, alkaline phosphatase, billirubin, urea nitrogen, glucose, cholesterol levels are measured. To specifically assess liver toxicity, analysis of amino transaminase (ALT), aspartate transaminase (AST), alkaline phosphatase (ALP), and total iron binding capacity (TIBC) in plasma are performed. As known, ALT, AST, and ALP are all enzymes associated with the liver and most often elevated (especially together) when the liver is damaged. TIBC measures blood iron levels assuming complete blood transferrin saturation. Transferrin is produced in the liver and, thus, lower TIBC indicates liver damage. At the end of each study after the animal is sacrificed, histology is performed on tissue from brain, liver, spleen, lung, bone marrow, lymph nodes, kidneys, and heart.

Pharmacokinetics and biodistribution are studied by intravenous injection. After I.A. injection MIONs rapidly join systemic circulation as if they were I.V injected, providing the fraction of the MION dose entrapped in the brain on the first pass is rather small (Eckman et al., J. Pharmacokinet. Biopharm. 1974; 2:257-85; herein incorporated by reference in its entirety). PK findings verify whether the two components remain attached in vivo in the absence of protamine. If the drug and MION are analyzed via separate means, such as by labeling the drug with a FITC and assessing MION with EPR, then a 1:1 ratio between these two markers in blood or excised tissues would indicate in vivo stability of the Drug/MION complex. A standard PK and biodistribution study is performed. Both the PE38 and ATF5-siRNA drug are labeled with FITC. Suspensions (100 μL) containing the Hep-MION/LMWP-Drug complexes are administered via a tail vein catheter to male Fisher 344 rats (250-300 g) at a dose of 12 mg Fe/kg body weight. At different time points (10, 20, 30, 60, 120, and 240 min and at 1, 2, 4, 7, 14, 28, and 60 days), blood samples (100 μL) is collected from the cannulated carotid artery into tubes spiked with heparin. Plasma fractions are collected by centrifugation and store at −80° C. immediately. Animals are then be sacrificed by exsanguinations, and organs of liver, spleen, lung, kidney, heart, and brain are removed immediately. The long duration of the experiments is because according to literature reports, MION clearance could last for a long time period ranging from 3 weeksl62 to 65 days (Lubbe et al., Cancer Res. 1996; 56:4694-701; herein incorporated by reference in its entirety). MION concentration is assayed by EPR whereas iron contents are be estimated by ICP, using a previously established procedure (Futami et al., J. Biosci. Bioeng. 2005; 99:95-103; herein incorporated by reference in its entirety). Drug concentrations are assayed by measuring the intensity of the FITC markers and then compared with ESR results to assess the in vivo stability of the Drug/MION complexes. Three rats are used for each time point, and 36 rats are included in each drug study.

### Example 4

### The Magnetophoretic Mobility and Superparamagnetism of Core-shell Iron Oxide Nanoparticles with Dual Targeting and Imaging Functionality

In this example, a MION drug carrier system with shell-core structure was synthesized and analyzed for magnetophoretic mobility and in vitro stability in 50% serum. The MION drug delivery system was also used for in vivo studies which showed that, through administration of the system using intra-arterial administration, first pass organ clearance was avoided and MION accumulation at the brain tumor site was enhanced nearly 35-fold.

Materials and methods for these experiments are detailed infra.

Materials and Methods

Chemicals

All materials were purchased and used as received without further treatment. Chemicals used include sodium citrate dihydrate (Fisher Scientific), glycine (Aldrich), ferrous chloride tetrahydrate (Fluka), iron chloride hexahydrate (Sigma-Aldrich), carboxymethyl dextran (CMD, Mr:14,400 Da, Fluka).

Preparation of Iron Oxide Nanoparticles

Preparation of Mion

MION was synthesized according to a modified procedure of Kim et al. (J. Magn. Magn. Mater. (2001) 225:30-36; herein incorporated by reference in its entirety). Briefly, a solution containing 0.76 mol/L of ferric chloride and 0.4 mol/L of ferrous chloride (molar ratio of ferric to ferrous was approximately 2:1) was prepared at pH 1.7 under N₂ protection. The iron solution was then added drop-wise into a 1.5 m NaOH solution under vigorous mechanical stirring. The reaction mixture was gradually heated (1° C./min) to 78° C. and held at this temperature for 1 h under stirring and N₂ protection. After separation of the supernatant using a permanent magnet, the wet sol was treated with 0.01 m HCl and then sonicated for 1 h. The acidified colloidal suspension of MION was filtered through 0.45 μm and then 0.22 μm membranes, followed by concentration to a suspension containing 0.7 mg Fe/ml using a Millipore (USA) ultrafiltration unit.

Preparation of Negatively-charged (nMION) and Positively-charged MION (pMION)

To 200 ml of 1 mg/ml sodium citrate, 200 ml of 0.7 mg Fe/ml iron oxide nanoparticles were added under stirring. The mixture was sonicated for 20 min and then further stirred for 2 h. After ultrafiltration to remove free sodium citrate, the concentration was adjusted to 0.35 mg Fe/ml. The resulting nanoparticles were negatively-charged (nMION). An analogous procedure was used to prepare glycine-modified, positively-charged nanoparticles (pMION).

CMD-coated Nanoparticles

To obtain self-assembled magnetic cores, an aqueous suspension of nMION was added to aqueous pMION under ultrasonification. An excess amount of pMION was used during the self-assembly process to obtain cores with net positive charges. These positively-charged large MION cores were then added into the same volume of CMD aqueous solution under mechanical stirring. The final CMD-coated nanoparticles were then separated from the free CMD polymer using magnetic separation.

Transmission Electron Microscopic (TEM) Images

Transmission electron microscopy (TEM) was conducted using a JEOL 3011 high-resolution electron microscope (JEOL Tokyo, Japan) operated at an accelerated voltage of 300 kV. Samples were prepared by applying diluted particle suspensions onto formvar film-coated copper grids (01813-F, Ted Pella, Inc, USA), followed by drying the grids at room temperature. Bright field (STEM-BF) imaging and X-ray energy dispersive spectroscopy (EDS) measurements were carried out on samples being placed on a carbon network coated copper grid, using a JEOL-2010F analytical electron microscope (AEM) operated in STEM mode at 200 kV. The lens conditions were set to define a probe size of 0.5 nm, and EDAX Genesis software was employed for EDS data collection and analysis.

Magnetization Measurement

Superparamagnetic properties were assessed at 25° C. using a superconducting quantum interference device (SQUID) (Quantum Design Inc., San Diego, Calif., USA). Iron content of nanoparticles was measured by inductively coupled plasma-optical emission spectroscopy (ICP-OES) on an Optima 2000 DV instrument (Perkin-Elmer, Inc., Boston, Mass., USA). Samples were spiked with yttrium internal standard and calibrated with water dilutions of an iron standard (GFS Chemicals, Columbus, Ohio).

DLS Size and Zeta Potential Measurement

Particle size and zeta potential were measured on a NICOMP 380 ZLS dynamic light scattering (DLS) instrument (PSS, Santa Barbara, Calif., USA) using a HeNe laser at 632 nm as the incident light. For size measurements, the volume-weighted distribution was obtained.

Thermogravimetric Analysis (TGA)

A thermogravimetric analyzer (TGA; TA Instruments Q50, New Castle, Del., USA), calibrated with nickel and alumel standards, was employed to determine the content of polymer coating of the nanoparticles. Samples were analyzed in a nitrogen atmosphere with a heating rate of 20° C./min.

Fourier Transform Infrared Spectroscopy (FTIR)

FTIR was performed on lyophilized samples after compression into ˜1 mm thick discs containing spectroscopic grade potassium bromide.

Magnetophoretic Mobility

Magnetophoretic mobility of the nanoparticles was measured by monitoring the change of nanoparticle concentration in water with time using a UV/Vis spectrophotometer (Beckman Du® 650 Spectrophotometer, USA) (as illustrated in FIG. 18). Briefly, nanoparticles were dispersed into a water solution and the suspension then placed into a 1 cm×1 cm quartz cuvette fixed with a sample holder. A permanent magnet (Dynal MPC®-L, Invitrogen) was placed along the side of the sample holder. The absorbance was monitored at a wavelength of 360 nm at which the absorbance was found to be linearly proportional to the iron content determined by ICP-OES. The magnetic field strength was measured using a 3-axis Hall Teslameter (THM 7025, GMW Associates). The gap between the magnet and nearest side of the cuvette was ˜5 mm-indicating the sample was located 5-15 mm from the magnet. All test samples were prepared at the same initial nanoparticle concentration.

Stability Assessment

Stability was assessed by dispersing the magnetic nanoparticle samples into a 0.1 m PBS buffer (pH 7.4) containing 50% calf serum. The suspensions were then placed into a 1 cm×1 cm quartz cuvette held by a sample holder, and the turbidity change monitored with time at 370 nm using a Beckman Du® 650 Spectrophotometer equipped with a thermostat at 37° C.

In Vivo Magnetic Resonance Images (MRI)

Magnetic targeting and MRI were performed (Chertok et al. (2008) Biomaterials 29:487-496; herein incorporated by reference in its entirety). Intracerebral 9L tumors were induced in male Fisher 344 rats (˜150 g) as described previously (Chertok et al. (2008) Biomaterials 29:487-496; herein incorporated by reference in its entirety). After the tumor size reached ˜70 μl, monitored and estimated by MRI, the animal was injected intravenously with the nanoparticle suspension at a dose of 12 mg Fe/kg. For magnetic targeting, the head of the animal was positioned between the poles of an electromagnet. The magnetic field density was adjusted to 0 T for control animals (without magnetic targeting) or 0.4 T for targeted animals for 30 min. MRI was conducted using a 12-cm horizontal-bore, 7 T Unity Inova imaging system (Varian, Palo Alto, Calif.), and T₂-weighted MRI images were acquired.

Results

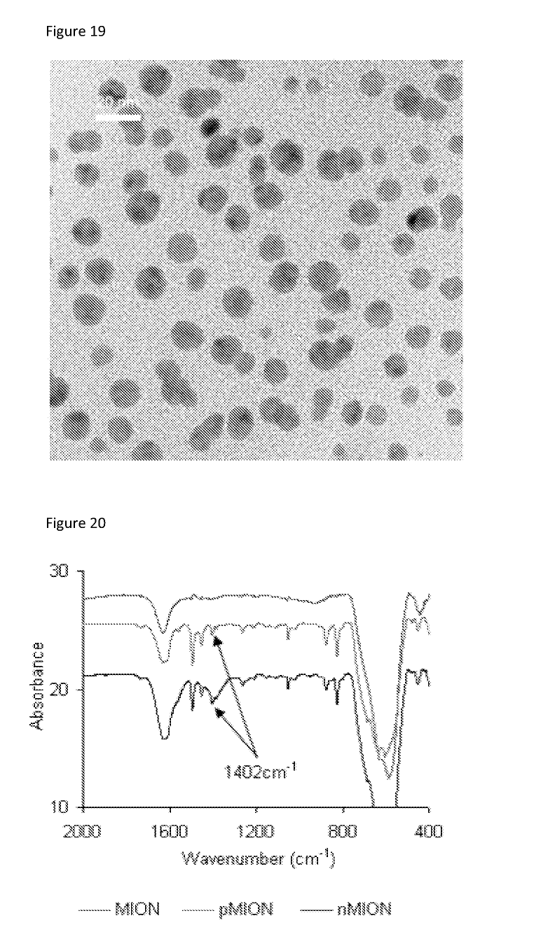

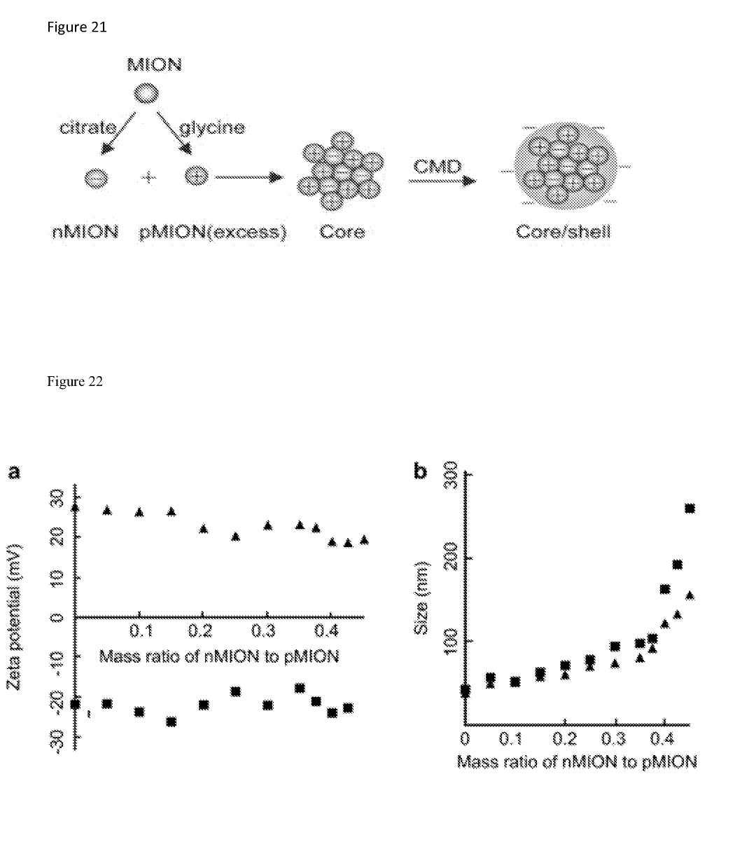

MION were first prepared using a modified protocol (Yu et al. (2010) J. Biomed Mater. Res. A 92:1468-1475; herein incorporated by reference in its entirety), based on Kim's method (Kim et al. (2001) J. Magn. Magn. Mater. 225:30-36; herein incorporated by reference in its entirety). TEM images (FIG. 19) showed that the average size of MION was 9±1.9 nm, whereas their average hydrodynamic size was determined to be 16.7 nm by the DLS method. In HCl medium at pH 4.0, these bare MION displayed a positive zeta potential of +29.7 mV, presumably originated from the superficial hydroxyl groups that were protonated in the acidic medium (Fauconnier et al. (1999) J. Mol. Liq. 83:233-242; Campos et al. (2004) Prog. Colloid Polym. Sci. 126:86-89; Hingston et al. (1967) Nature 215:1459-1461; each herein incorporated by reference in its entirety). Treating these bare MION with either sodium citrate or glycine yielded negatively- or positively-charged nanoparticles (nMION, pMION), with zeta potentials being changed to −19.4 mV and +34.4 mV, respectively. Both nMION and pMION displayed a new peak at 1402 cm⁻¹ in FTIR, compared with the original un-modified or bare MION (FIG. 20). This new peak was assigned to the symmetric stretching band of two-oxygen coordination of the carboxylate group with iron atom (Yu et al. (2004) J. Mater. Chem. 14:2781-2786; herein incorporated by reference in its entirety), providing evidence that a polar covalent bond of Fe—O—C (the C in the COO⁻ group of citrate or glycine) linkage was formed (Harris et al. (2003) Chem. Mater. 15:1367-1377; herein incorporated by reference in its entirety). Self-assembly of nMION and pMION via electrostatic interaction yielded a large-size magnetic core, which carries a positive surface charge when an excess amount of pMION is applied (FIG. 21). The carboxylate group carrying polymer, carboxymethyldextran (CMD), would was then readily coated onto the large MION cores to produce magnetic nanoparticles with the desired core-shell structure.

FIG. 22A showed that after coating the aforementioned self-assembled, large-size MION core by CMD, the zeta potential of the coated nanoparticles sharply switched to a negative value, due to the presence of free carboxylate groups from CMD on the particle surface. Both the sizes of the MION core and the CMD-coated nanoparticles could be readily regulated by altering the mass ratio between the pMION and nMION in the self-assembly process (FIG. 22B). A series of CMD-coated magnetic nanoparticles with average hydrodynamic sizes ranging from about 40 to 280 nm were successfully synthesized using this procedure.

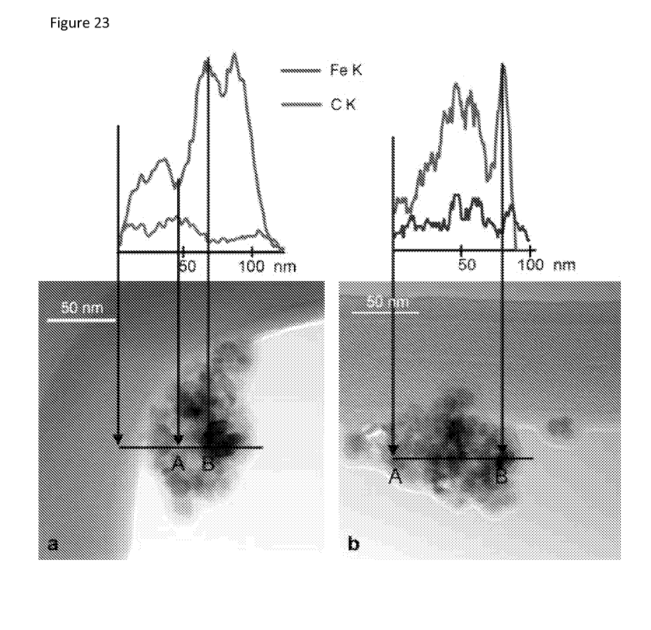

The core-shell structure of CMD-coated particles was verified by utilizing a variety of methods including scanning transmission electron microscopy (STEM), X-ray energy dispersive spectroscopy (EDS) scan, and thermogravimetric analysis (TGA). All of the analyses were performed on nanoparticles of approximately 100 nm in size (termed CMD-coated 100), and data were compared with two existing commercial products, fluidMAG-CMX (CMD-coated, 100 nm) and fluidMAG-D (starch-coated, 100 nm) supplied by the Chemicell Company. Results from the bright field images (STEM-BF) demonstrated a shell-like structure of the polymer coating on these nanoparticles (FIG. 23). Thickness of the polymer shell for CMD-coated100 was estimated to be about 2.7 nm. To further verify the core-shell structure in the synthesized nanoparticles, confirmation was desired that no polymer exists within the particle interior. Hence, element analyses of both carbon and iron contents was carried out by using EDS linescan spectroscopy along a linear path through the particle. If the nanoparticle is comprised of a core-shell structure and the core composed purely of iron oxide, the carbon content would not vary, while the iron content increases, along the radius toward the particle center. On the other hand, if polymer was somehow incorporated into the interior part of the particles, both carbon and iron content should increase gradually toward the particle center. As shown in FIG. 23A, the iron content (red curve) of CMD-coated 100 increased gradually along the line toward the center. It is consistent with pixel brightness observed from the STEM-BF images Taking area-B in FIG. 23A as an example, it was relatively darker in the images, indicating the presence of high iron content. Conversely, the carbon content (shown in FIG. 23A) remained nearly constant and did not exhibit significant difference at area-B compared with those close to the two edges (at the 25- and 100-nm positions on the scan line) of the nanoparticles. While the present invention is not limited to any particular mechanism, and an understanding of the mechanism is not necessary to practice the present invention, it is contemplated that the CMD polymer resided primarily on the particle surface; otherwise the change in carbon content should be in accordance to that in iron content.

FluidMAG-CMX displayed a distinctive pattern concerning the content ratio between carbon and iron along the EDS linescan. As seen in FIG. 23B, both carbon and iron contents increased along the line toward the particle center, indicating that the polymer in fluidMAG-CMX was significantly incorporated into the interior part of the particles.

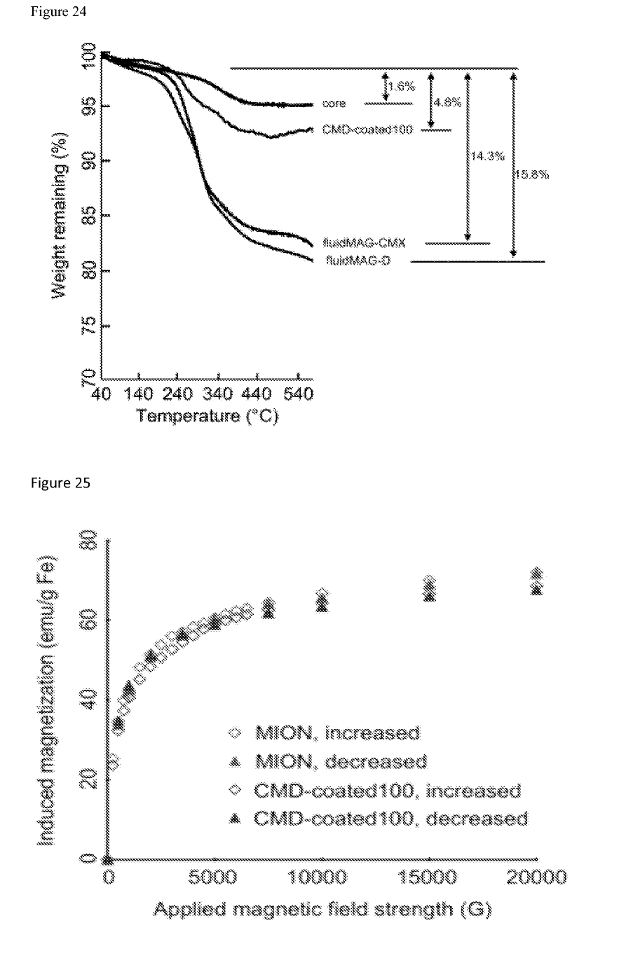

FIG. 24 revealed the TGA results for these magnetic nanoparticles. A trivial weight loss of 1.6%, originating from thermal removal of the citrate and glycine coating, was observed from the MION core formed by self-assembly of nMION and pMION. CMD-coated 100 sustained a weight loss of 4.8%, due to the additional CMD coating the particle. Both fluidMAG-CMX and fluidMAG-D underwent weight losses of 14.3% and 15.8%, respectively. As calculated, CMD-coated 100 consisted of only 30% of the polymer content of fluidMAG-CMX (4.8% versus 14.3%, respectively) after normalization by iron content. Since the shell coating contains almost the same polymer content, the significantly low polymer content in CMD-coated100 further confirmed the applicability of the described method in producing a shell-core type of nanoparticles. The dearth of polymer matrix within the interior part of particles enhances the mobility of the nanoparticles under the influence of a magnetic field.

The SQUID results, shown in FIG. 24, demonstrated that both MION and CMD-coated 100 exhibited superparamagnetic properties, without remnant hysteresis loop observed in the magnetization/demagnetization curves. The saturation magnetism for CMD-coated 100 was estimated to be 69.4 emu/g Fe, slightly lower than the value of 73.3 emu/g Fe obtained for the MION.

FIG. 12 demonstrated that CMD-coated 100 moved significantly faster, under a magnetic force, than either of the two commercial products. The magnetophoretic mobility was quantitatively compared by using the migration half-time (t_{1/2}) as the indicator, which was defined as the time required to reduce the turbidity by 50%. The t_{1/2} of CMD-coated 100 was about 40 min, which was an approximately 3- to 4-fold enhancement of the mobility compared to the values of 125 min and 155 min for fluidMAG-CMX and fluidMAG-D, respectively.

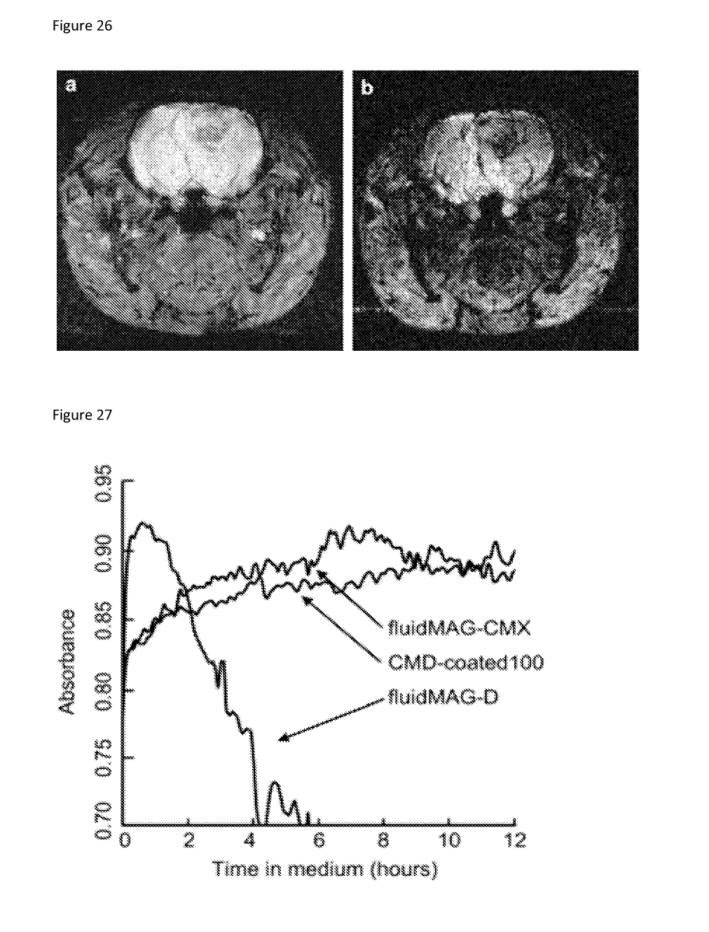

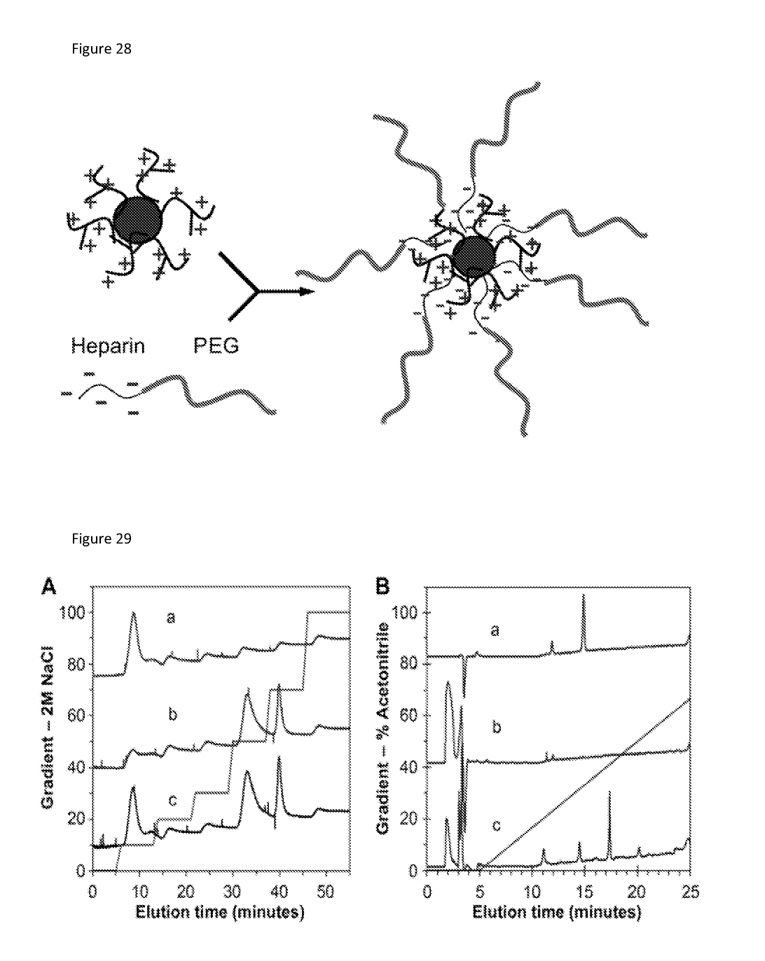

Animal study experiments were conducted as detailed herein to demonstrate the utility of CMD-coated 100 as a MRI contrast enhancer for monitoring brain tumors, using rats harboring brain glioma (Chertok et al. (2008) Biomaterials 29:487-496; herein incorporated by reference in its entirety). As a control, the rat was treated with the same dose of CMD-coated 100 without targeting via the magnetic force. As seen in FIG. 26, the post-injection image of the non-targeted control animal (FIG. 26A) displayed a very slight signal reduction within the glioma lesion. In contrast, GE images of the targeted animal acquired 30 min post-injection (FIG. 26B/FIG. 8b) exhibited a region of pronounced hypointensity, implicating the accumulation of magnetic nanoparticles inside the tumor.

To assess stability, the above three magnetic nanoparticles (CMD-coated 100, fluidMAG-CMX, and fluidMAG-D) were exposed to a simulated body fluid containing 50% (v/v) calf serum in 0.1 m PBS (pH 7.4) and the turbidity monitored at 370 nm as an indication of particle aggregation. As seen in FIG. 27, the turbidity of fluidMAG-D reached a summit at the 40-min mark and then dropped rapidly thereafter. This drop was due to the occurrence of precipitation of the large aggregates. The precipitate could be visually detected after 1.5 h of exposure to the simulated body fluid. The turbidity of both CMD-coated 100 and fluidMAG-CMX increased slowly and then remained at a plateau till the 12-h mark after exposure to the medium. In addition, no precipitate was ever visually observed during this 12-h period of incubation.

Superparamagnetic properties of iron oxide particles can be achieved when the particle size is below a critical domain size (Jeong et al. (2007) Adv. Mater. 19:33-60; Tartaj et al. (2003) J. Phys. D. Appl. Phys. 36:182-197; Frenkel et al. (1930) 126:182-197; Sun et al. (2008) Adv. Drug Deliv. Res. 60:1252-1265; each herein incorporated by reference in its entirety). The magnetophoretic mobility, which benefits the targeting efficiency when the particles are utilized as a vehicle for drug delivery, increases with the size.

TEM, EDS linescan, and TGA have provided proof of the core-shell structure. The TGA results were found to be highly consistent with the EDS spectra. Based on the peak areas of carbon and iron elements in EDS results, the C/Fe ratios were estimated to be 0.050 and 0.158 for CMD-coated100 and fluidMAG-CMX, respectively. In agreement with the TGA findings, comparison of these two numbers yielded a 31% ratio of the carbon content or, in other words, of the polymer content between CMD-coated 100 and fluidMAG-CMX. This is almost identical to the ratio of 30.2% obtained from the TGA results. This agreement between TGA results with X-ray energy dispersive spectroscopy (EDS) demonstrated reliability of both analyses.

Additionally, the polymer content of CMD-coated100 could also be calculated as 4.2 wt % with assumption of a shell-core structure. This value is very close to the experimental weight loss of 4.8% measured by TGA.

Instead of a polymer, small molecules such as citrate or glycine were adopted to create surface charges on MION for production of nMION and pMION (Yu et al. (2010) J Biomed. Mater. Res. A 92:1468-1475; Fauconnier et al. (1996) J. Liq. Chromtogr. R 19:783-797; Laaksonen et al. (2009) Langmuir 25:5185-5192; Yu et al. (2009) Biometerials 30:4716-4722; each herein incorporated by reference in its entirety). These charged nanoparticles remained under the size threshold essential for retaining superparamagnetic behaviors.

The self-assembly technology was optimized. Low concentrations of nMION or pMION were employed, for instance, to lower the probability of the occurrence of precipitation. Ultrasonication is also important to maintaining uniform size as it assists the dispersion and homogenization of freshly added nMION. Addition order of nMION into pMION was specifically taken as well. Since it was desirable to produce a core of net positive charge, if pMION were added to nMION, the net charge in the system would have to cross from negative to positive, or pass the charge balance point, at which point it would precipitate.

The size of both cores and coated particles could be readily regulated by simply varying the ratio of pMION to nMION (FIG. 22B). When adding nMION into pMION, positively-charged pMION self-assemble around nMION via electrostatic interaction, resulting in size increase. As more nMION is added, larger-sized, positively-charged particles assemble around the nMION further and result in sharp size increase. An inflection point was observed in the size variation curve (FIG. 22B). With the further increase in the ratio of nMION to pMION, the net positive charge in the system decreases and is not strong enough to stabilize the large particles. Ultimately, precipitation occurred. Thus this technique provided a convenient route for the synthesis of magnetic nanoparticles of controlled size.

CMD was chosen as the coating material. The CMD coating has abundant carboxylate groups on the polymer that provide functional anchors for subsequent conjugation with drug molecules or biological agents.

The effect of self-assembly and the coating process on magnetic properties was determined. For a superparamagnetic material, the demagnetization curve normally follows the magnetization profile closely with no remnant magnetization being observed (Kim et al. (2007) J Appl. Phys. 101:09 M516.1-09M516.3; herein incorporated by reference in its entirety); an important property in achieving MRI and magnetic-mediated tumor targeting. This observation (FIG. 25) indicated that all the processes involved in the synthesis, including MION surface modifications, self-assembly of the +/− MION, and polymer coating did not alter the superparamagnetic behavior of the produced magnetic nanoparticles.

The magnetophoretic mobility of these nanoparticles was comparatively assessed by an innovative method designed here. The turbidity of the suspension was monitored using a spectrophotometer at the wavelength of 360 nm. The quicker the nanoparticles migrated under the influence of the magnetic field, the faster the turbidity decreased. The three test nanoparticles in FIG. 12 all possessed similar hydrodynamic sizes. Therefore, the superior mobility of CMD-coated 100 was attributed primarily to the core-shell structure. While the present invention is not limited to any particular mechanism, and an understanding of the mechanism is not necessary to practice the present invention, it is contemplated that the specifically designed synthesis strategy in producing core-shell nanoparticles would minimize polymer embedding within the interior part of the particles, and consequently maximize the response of these particles toward the magnetic field.

The CMD-coated 100 exhibited superior stability upon storage. It was noted that CMD-coated100 and fluidMAG-CMX were made by the same coating material, negatively-charged carboxymethyldextran.

### Example 5

### In Vivo Magnetic Brain Tumor Targeting

### A. Materials and Methods

### Synthesis and Purification of LMWH-PEG Conjugate

LMWH-PEG was synthesized using a two-step process. First, heparin (Grade IA, Sigma) was partially depolymerized to form an aldehyde-containing terminal anhydromannose residue. Activated LMWH oligosaccharides were then coupled to amine-bearing polyethylene glycol (mPEG-NH₂) by reductive amination.

### Preparation of Aldehyde-Bearing Lmwh (Lmwh-Cho)

Nitrous acid cleavage of heparin was carried out as previously described (Liang et al., AAPS PharmSci 2000; 2(1):E7) with minor modifications. Briefly, heparin (0.5 g) was dissolved in 150 ml of Milli-Q (Millipore, Billerica, Mass.) deionized water (DW) and the solution cooled on ice. Sodium nitrite (NaNO₃, 20 mg) was added to the solution, and 0.5 M HCl then used to adjust the solution to pH 2.7. The reaction mixture was stirred on ice for 2 h and then the reaction terminated by adjusting the pH to 7.0 with NaOH. Heparin depolymerization products were fractionated using two consecutive ultrafiltration steps with 3 kDa and 5 kDa cut-off membranes to screen for oligosaccharides with the desirable molecular weight of 3-5 kDa. The lyophilized product was analyzed for presence of aldehydes using the dinitrosalicylic acid method [20] and was found to contain 0.25 mmol aldehydes/mg heparin, equivalent to about one aldehyde group per heparin chain.

Coupling of LMWH-CHO and mPEG-NH₂

Coupling of the aldehyde-bearing LMWH-CHO and primary amine-bearing mPEG-NH₂ (10 kDa, Nektar Therapeutics, Huntsville, Ala.) was carried out by reductive amination. Briefly, LMWH-CHO (20 mg, 5 mmol CHO) and mPEG-NH₂ (15 mmol) were dissolved in 10 ml borate buffer (50 mM, pH 9.5). Sodium cyanoborohydride (NaCNBH₃, 250 mmol) was then added to the reaction mixture for in situ reduction of the imine bond (Borch et al., J Am Chem Soc 1971; 93:2897-905). After 24 h of incubation at 37° C., additional 250 mmol of NaCNBH₃ was added and the solution was stirred for another 24 h.

### Purification of LMWH-PEG Conjugate

The conjugate was purified in three steps. First, the reaction mixture was subjected to ultrafiltration with 5 kDa cut-off membrane (Millipore) to remove unreacted LMWH. The concentrated retentate was then further purified from unreacted PEG by anion-exchange with a High-Q column (BioRad Laboratories, Hercules, Calif.). The elution was performed with 0-2 M NaCl step-gradient at a flow rate of 1 ml/min and followed by UV detection at 214 nm. The fraction eluted with 1 M NaCl (the salt concentration required for elution of LMWH) was collected for further purification. This fraction was loaded on an Altima C8 (250×4.6 mm, 5 μm) reverse phase column equilibrated with 0.1% trifluoroacetic acid in DW. The remnants of the unreacted LMWH were removed with the flow through at a flow rate of 1 ml/min and the column-adsorbed species were eluted with acetonitrile gradient (0-70% acetonitrile in DW). The fraction desorbed from the column with 40% acetonitrile in DW was collected. After solvent evaporation, the residue was redissolved in DW and analyzed for the presence of Heparin and PEG using Azur A (Copley et al., J Lab Clin Med 1943; 28:762-70) and Barium-Iodine (Sims et al., Anal Biochem 1980; 107(1):60-3) spectrophotometric assays, respectively.

### GPEI/LMWH-PEG Complexation Studies

Cationic magnetic nanoparticles were prepared by attaching polyethyleneimine (PEI) chains to pendant carboxylic groups of fluid MAG-ARA magnetic nanoparticles (Chemicell, Germany) using the well-established EDC-coupling method (Futami et al., J Biosci Bioeng 2005; 99(2):95-103). The modified particles were termed GPEI. Complexes were formulated by mixing GPEI solutions (3 mg Fe/ml, 50 mL) with LMWH-PEG conjugate or free LMWH at 0-6.4% w/w (LMWH/GPEI). Resulting solutions were diluted to 200 mL with deionized water (DW) and stirred at RT for 15 min. GPEI/LMWH-PEG complexes were removed from solution with a magnetic separator. Isolated complexes were resuspended in DW and analyzed for particle size distribution and zeta potential using Nicomp 380 particle sizer (Nicomp, Santa Barbara, Calif.). Residual amounts of free conjugates in the supernatant were determined with the Azur A assay (Copley et al., supra).

### Evaluation of Nanoparticle Stability In Vitro

Size stability of conjugate-protected and free GPEI nanoparticles was studied as a function of time in protein-rich medium using dynamic light scattering (Nicomp, Santa Barbara, CA). GPEI complexes with LMWH or LMWH-PEG, formulated as described above at 0-6.4% w/w LMWH/GPEI, or free GPEI, were mixed with reduced serum medium (2 ml, Opti-Mem I, Invitrogen). Samples were incubated in a cuvette at ambient temperature and particle size distribution was analyzed at 5 min intervals for 30 min.

### Pharmacokinetic Analysis

All animal experiments were conducted according to protocols approved by the University of Michigan Committee on Use and Care of Animals (UCUCA). The pharmacokinetics of GPEI and GPEI/LMWH-PEG complexes were studied in male Fisher 344 rats weighting 200-250 g (n¼4 for each groups). The nanoparticles were administered intravenously via tail vein and blood samples taken through the cannulated carotid artery. All animals were initially anesthetized by intraperitoneal injection of ketamin/xylazine mixture (87/13 mg/kg body weight). The left carotid artery of the animals was exposed by blunt dissection and ligated rostrally to occlude the flow. Polyethylene tubing (PE-10, BD Corp., Franklin Lakes, N.J.) was inserted caudally via a small incision in the arterial wall and secured in place by ligation. The intracarotid catheter was flushed with Heparin flush solution (Hepflush-10, 10 USP Units/ml, Abraxis Pharmaceutical products, IL) and clamped. Tail veins of the animals were cannulated with a 26-gauge angiocatheter (Angiocath, BD Corp., Franklin Lakes, N.J.). The nanoparticle suspension in PBS was administered to rats via the tail vein catheter at a dose of 12 mg Fe/kg body weight. Blood samples (100 mL) were collected from the cannulated carotid artery in Eppendorf tubes (0.5 ml) spiked with Heparin solution (10 mL, 5000 USP Units/ml). Samples were acquired before and serially after nanoparticle administration at preset time intervals for 30 min. Plasma fractions were immediately separated by centrifugation (3 min at 7000 g) and stored at −80° C.

### Tumor Implantation

Intracerebral 9L tumors were induced in male Fisher 344 rats weighting 125-150 g according to a previously described procedure (Ross et al., Proc Natl Acad Sci USA 1998; 95:7012-7). Briefly, rat 9L-glioma cells (Brain Tumor Research Center, University of California, San Francisco) were culturedin Dulbecco's modified Eagle's medium (DMEM) supplemented with 10% heatinactivated fetal bovine serum, 100 IU/mL penicillin, 100 mg/mL streptomycin and 0.29 mg of L-glutamine at 37° C. in a humidified atmosphere of 5% CO₂. Prior to implantation, cells were grown to confluency in 100 mm culture dishes, harvested and resuspended in serum free DMEM at a concentration of w105 cells/mL. A 1 mm hole was drilled in the right skull of the animals, 1 mm anterior to the bregma and 5 mm lateral to the midline. The cell suspension (10 mL) was then implanted at a depth of 3 mminto the right forebrain through the burr hole. The surgical field was cleaned with 70% ethanol and the burr hole was filled with bone wax (Ethicon Inc., Summerfield, N.J.) to prevent extracerebral extension of the tumor. The tumor volume of the animals was monitored with MRI beginning on day 10 after cell implantation to select tumors between 70 and 90 mL for magnetic targeting experiments.

### Magnetic Targeting

Magnetic targeting was carried out according to a previously reported procedure (Chertok et al., Biomaterials 2008; 29(4):487-96). Briefly, anaesthetized animals were positioned on a platform with their head subjected to 0.4 T magnetic field. GPEI or GPEI/LMWH-PEG complexes were administered intravenously at a dose of 12 mg Fe/kg via the cannulated tail vein (n¼4 for each group). Animals were retained in the magnetic field for 30 min after nanoparticle administration.

### Magnetic Resonance Imaging (MRI)

MRI experiments were performed on an 18 cm horizontal-bore, 7 Tesla Varian Unity Inova imaging system (Varian, Palo Alto, Calif.). Animals were anesthetized with 1.5% isoflurane/air mixture and imaged using a 35 mmdiameter quadrature RF head coil (USA Instruments Inc, OH). Animals were maintained at 37° C. inside the magnet using a thermostated circulating water bath. To monitor nanoparticle accumulation in brain tumors, transverse relaxation rate maps (R2) were constructed according to a previously reported procedure (Chertok et al., supra). Briefly, T2-weighted MRI scans of rat brain were acquired using a multi-slice fast spin echo sequence with the following parameters: repetition time (TR)=4 s, field of view=30×30 over 128×128 matrix, slice thickness=1 mm, slice separation=2 mm, number of slices=13, four signal averages per phase encoding step. Two consecutive sets of T2-weighted images with effective echo time (TE) of either 30 or 60 ms were collected before administration of nanoparticles (pre-scans) and immediately after magnetic targeting. R2 relaxation rate maps were calculated from resulting signal intensities using the following equation:

$${R}_{2} = \frac{\text{?}}{\text{?}} = \frac{ln [ {S}_{1} ( \text{?} ) / {S}_{2} ( \text{?} ) ]}{\text{?} - \text{?}}$$

$$\text{?} \text{indicates text missing or illegible when filed}$$

where S₁(TE₁) and S₂(TE₂) are the signal intensities acquired with effective echo times TE₁ and TE₂, respectively. Image analysis was performed with Matlab 7.1 software (The MathWorks, MA). To infer the nanoparticle accumulation within tumor lesions, tumor-circumscribing regions of interest (ROIs) were manually drawn on the R₂ maps. Mean signal intensities of the ROIs were calculated to compare transverse relaxation rates before nanoparticle administration and after magnetic targeting. The change in R₂ relaxation rate caused by the presence of nanoparticles within the outlined ROIs was expressed as a percent change of the baseline R₂ value.

### EPR Analysis

Nanoparticle concentrations were determined by EPR spectroscopy as previously reported (Chertok et al., supra). Briefly, ESR spectra of the samples were acquired using an EMX ESR spectrometer (Bruker Instruments Inc., Billerica, Mass.) equipped with a liquid nitrogen cryostat. The acquisition parameters were: resonant frequency: w9.2 GHz, microwave power: 20 mW, temperature: 145 K, modulation amplitude: 5 G and receiver gain of 5×10⁴ and 5×10³ for tissue and plasma samples, respectively. The double integral of the ESR spectra of tissue/plasma samples was calculated to quantify the nanoparticles. Calibration curves were constructed with nanoparticle solutions of known iron concentrations. The data were corrected for the background tissue absorption using control tissue samples from the animals not exposed to the nanoparticles or plasma samples collected prior to the nanoparticle injection. The area under the plasma concentration versus time profiles (AUC) was estimated numerically by a linear trapezoidal integration method.

### Statistical Analysis

Data are presented as mean±SD, unless indicated otherwise. Nanoparticle concentrations in tumor and contra-lateral brain tissues of complex-protected and non-protected GPEI were compared using unpaired student t-test. A p-value of <0.05 was considered statistically significant.

### B. Results

### Preparation and Characterization of LMWH-PEG Conjugate

LMWH-PEG conjugates were synthesized by end-to-end, site specific attachment of LMWH to PEG. This procedure was specifically selected in order to avoid the loss of carboxyl and sulfate moieties along the heparin backbone. To generate a terminal attachment site on the heparin molecules, unfractionated heparin was first partially depolymerized with nitrous acid to form LMWH with a 2,5-anhydromannose terminal residue. This residue possesses a reactive aldehyde group, which was used to couple LMWH to the primary amine of mPEG-NH₂ by reductive amination. The resulting conjugate was purified using several sequential steps. Purification chromatograms are shown in FIG. 29. As evident from FIG. 29A, free PEG (FIG. 29A: a) exhibited only weak association with the anion-exchange (═N⁺(CH₃)₃) matrix and could be eluted with 0.2 M NaCl. Free LMWH (FIG. 29A: b) was found to bind strongly to the cationic matrix and its elution required 1 M NaCl. Thus, anion-exchange step was used to remove the free PEG from the mixture of LMWH-PEG and remnants of free LMWH (FIG. 29A: c). Subsequent C8 reverse phase separation (FIG. 29B) was used to ensure removal of free LMWH remnants from the conjugate solution. Hydrophilic LMWH did not bind to the C8 matrix (FIG. 29B: b) and thus eluted with the flow through. The PEG-containing conjugate, purified of free PEG, exhibited affinity to the C8 matrix (FIG. 29B: c). Overall, the chromatograms of FIG. 29 qualitatively reveal formation of the LMWHPEG species. Furthermore, the molar ratio of LMWH to PEG in the purified conjugate was found to be approximately 1:1, further confirming successful end-to-end coupling.

### In Vitro GPEI/LMWH-PEG Complexation and Stability Analysis

GPEI iron oxide nanoparticles, surface-modified with short polyethyleneimine (PEI-MW˜1200 Da) chains, were chosen as the model to represent cationic magnetic nanocarriers. GPEI exhibited a zeta potential of b25.2 mV and hydrodynamic diameter of 228 nm (polydispersity, PDI=0.2), as assessed by dynamic light scattering.

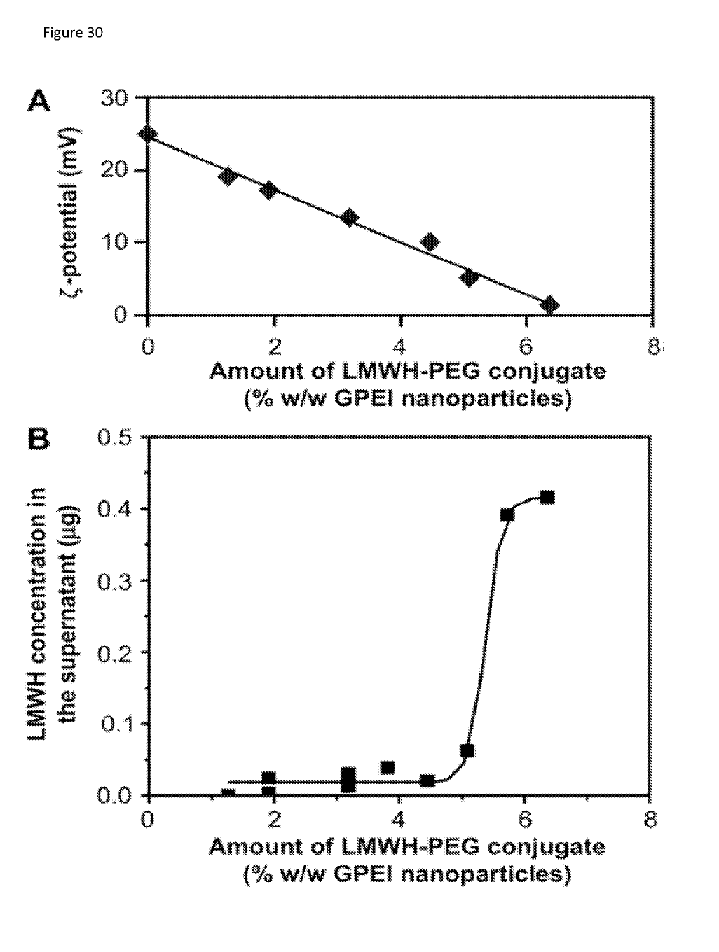

Complexation of GPEI nanoparticles with the LMWH-PEG conjugate was examined at different weight ratios of these two components. Properties of the nanoparticle surface were assessed by utilizing two techniques: 1) determination of ζ-potential, and 2) analysis of the residual amount of free conjugate in the supernatant following removal of GPEI/LMWH-PEG complexes from solution with a magnetic separator. As seen in FIG. 30, zeta potential of the nanoparticles exhibited an inverse linear dependence on the amount of added conjugate (R2=0.98, FIG. 30A), indicating electrostatic adsorption of the anionic LMWH-PEG to the cationic GPEI nanoparticle surface. For example, addition of the conjugate at 3.2% w/w (LMWH/GPEI) resulted in two-fold reduction in surface charge (ζ=+13.4 mV) compared with free GPEI (ζ=+25.2 mV). In agreement with the ζ-potential data, amounts of residual conjugate in the supernatant were found to increase with increasing amount of added conjugate (FIG. 30B). Steadily low amounts of surplus conjugate were detected in the supernatant with addition of conjugate to GPEI up to a ratio of about 5% w/w. Further addition up to about 6% w/w ratio resulted in an abrupt increase of conjugate level in the supernatant, corresponding well with charge neutralization of the GPEI surface (ζ=+1.3 mV) and indicating saturation of the GPEI surface with the charge masking LMWH-PEG. Complexation did not significantly alter the hydrodynamic diameter of conjugate-masked GPEI in deionized water (243 nm, PDI=0.1) from that of free GPEI (228 nm, PDI=0.2).

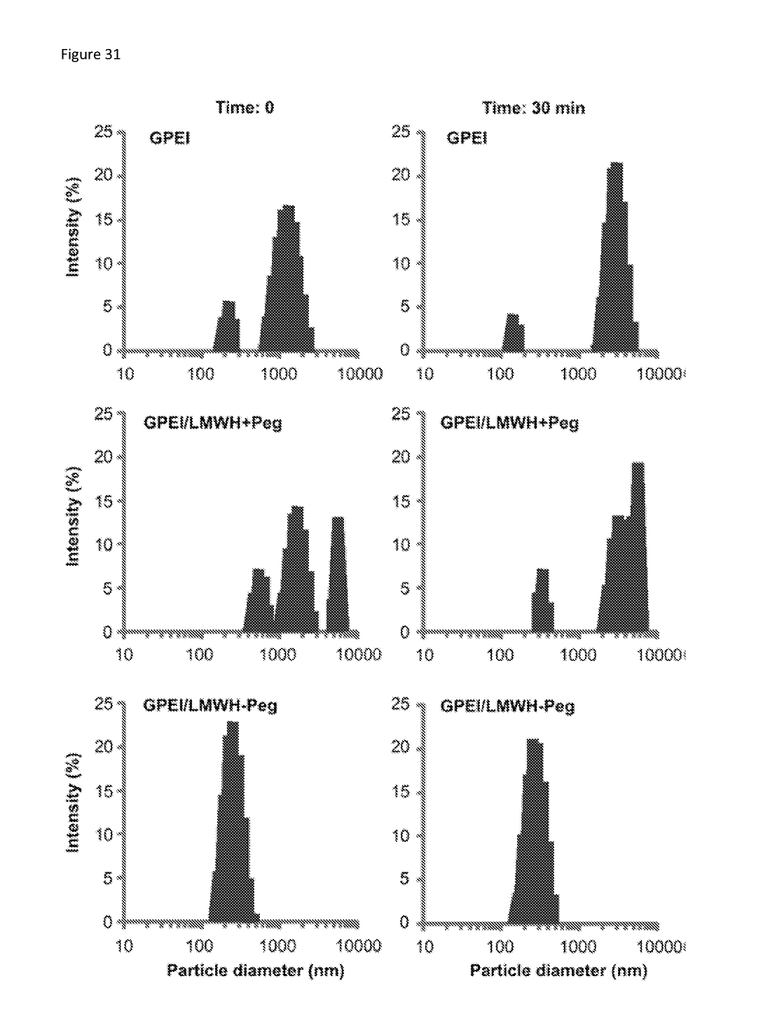

To examine whether complexation with the LMWH-PEG conjugate would yield protection to the positively charged GPEI nanoparticles in a protein-rich environment, in vitro stability of the complexes, and free nanoparticles, were evaluated in medium containing reduced serum (Opti-Mem I, Invitrogen). Complexes of varying GPEI/LMWH-PEG ratios were incubated with the medium and changes in particle size were monitored over time using dynamic light scattering. As displayed in FIG. 31, native, non-protected GPEI particles were prone to aggregation and, after only 5 min of incubation with the medium, particle size shifted from about 228 nm (GPEI diameter in water) to the micron range. Complexation of the GPEI with free LMWH and PEG, at individual concentrations equivalent to that in the 3.2% w/w LMWH/GPEI complex, did not reverse the aggregation pattern observed for the unprotected GPEI (middle lane of FIG. 31). Nanoparticles complexed with the LMWH-PEG conjugate at concentrations of 3.2% w/w (LMWH/GPEI) and higher maintained their original size distribution in the protein-rich medium for at least 30 min. Hydrodynamic diameters determined for 3.2% w/w (LMWH/GPEI) complexes were 243 nm (PDI=0.13) and 265 nm (PDI=0.15) after 5 and 30 min of incubation in the medium, respectively.

### In Vivo Pharmacokinetics and Tumor Accumulation of Complex-masked GPEI

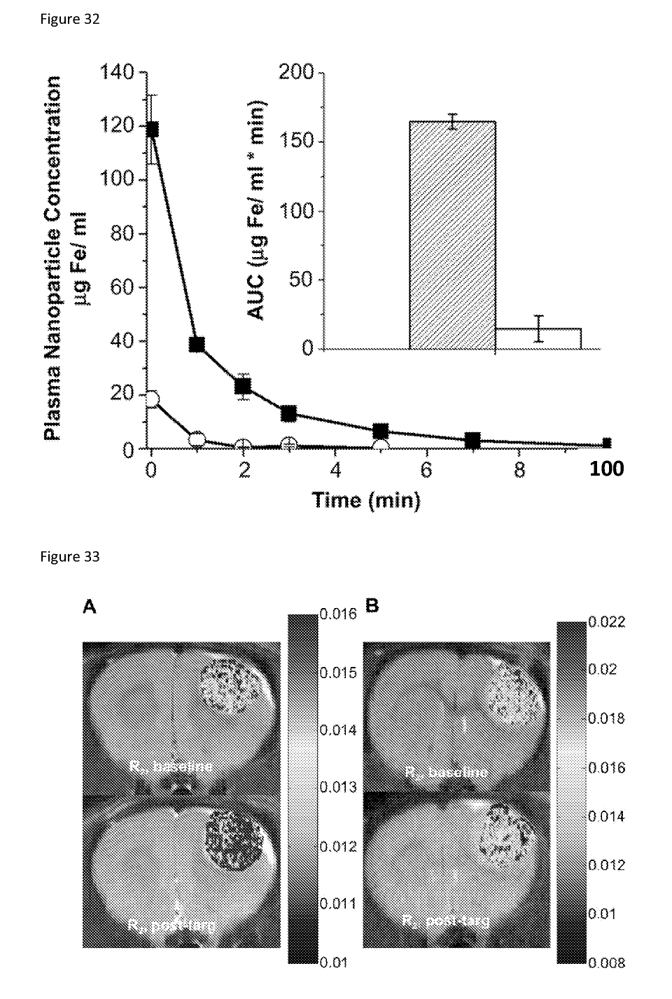

The pharmacokinetic behavior of the complexes was examined in vivo. The 3.2% weight ratio of LMWH-PEG and GPEI was used for complex formulation in pharmacokinetic studies because of the previously observed in vitro stabilization effect. Analysis of nanoparticle concentration in plasma samples following intravenous administration revealed that complexation with LMWH-PEG significantly improved the pharmacokinetic profile of GPEI (FIG. 32), as the plasma AUC (FIG. 32; inset) for GPEI/LMWH-PEG complexes (164.8±5.5 mg Fe/mlmin) was found to increase by 11-fold (p<0.001) over that of the native, nonprotected GPEI particles (14.8±9.3 mg Fe/ml min).

It was next assessed whether improvement in AUC could enhance magnetic entrapment of the conjugate-protected GPEI in tumor lesion of glioma-bearing rats. Accumulation of magnetic nanoparticles in tumor lesions was monitored with magnetic resonance imaging (MRI). Iron oxide nanoparticles are known to enhance transverse relaxation rate of protons (R₂), and thus manifest their presence at a given spatial location by a local increase in R₂. R₂ maps were calculated from sets of T₂-weightedMRl images, acquired before nanoparticle administration and after magnetic targeting. FIG. 33 presents representative R₂ maps of glioma regions of interest (ROIg) overlayed on anatomical T₂-weighted brain images for rats administered with GPEI and GPEI/LMWH-PEG complexes. In the group of complex-protectedGPEI (FIG. 33A), a 15.2±2.5% increase in R₂ mean pixel intensity above the baseline value was observed within the ROIg after magnetic targeting reflecting nanoparticle accumulation in the tumor lesion. In contrast, a mean R₂ change of only 1.3±0.7% was seen in tumors of rats administered with the native, non-protected GPEI (FIG. 33B). No significant difference (p=0.121) in the baseline mean R₂ values of the ROIg was observed between the groups administered with the complex-protected (14.3±0.8 s⁻¹) and the non-protected GPEI (15.3±0.9 s⁻¹).

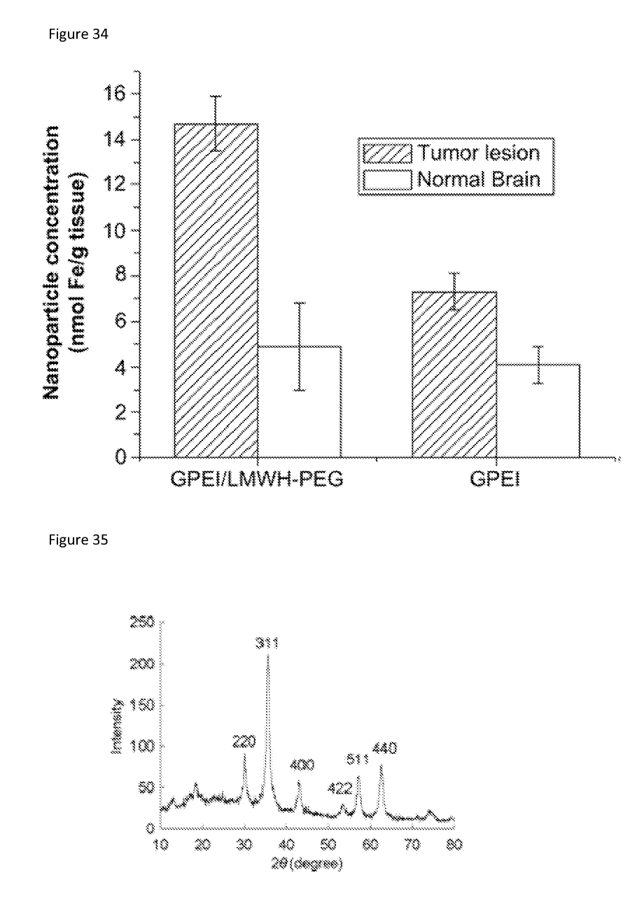

To quantify the extent of nanoparticle accumulation in the brain of GPEI and GPEI/LMWH-PEG injected rats, excised tissue samples of tumor and normal brain were assayed for nanoparticle concentration. As evident from FIG. 34, a 2-fold higher nanoparticle concentration (p<0.01) was detected in tumor lesions of rats administered with GPEI/LMWH-PEG complexes (14.7±1.2 nmol Fe/g tissue) compared to those injected with the non-protected GPEI (7.3±0.8 nmol Fe/g tissue). This increase in surface-protected nanoparticle accumulation in the tumor of GPEI/LMWH-PEG administered rats was not accompanied by any detectable increase in the normal brain tissue, as accumulation in the contra-lateral brain was not significantly different between the surface-protected and the non-protected GPEI groups (p=0.617). Surface protection also resulted in an improved targeting selectivity of nanoparticles towards the tumor lesion.

### Example 6

### Gum Arabic-Coated Magnetic Nanoparticles for Simultaneous Magnetic Targeting and Tumor Imaging

### Materials and Methods

### Materials

Unless otherwise stated, FeCl₂.4H₂O (>99%), FeCl₃.6H₂O (>99%), NH₃.H₂O(NH₃ content, 28˜30%), GA, 2-[N-morpholino]ethane sulfonic acid (MES), 1-ethyl-3-(3-dimethylaminopropyl) carbodiimide hydrochloride (EDC), and all the other chemicals and solvents were purchased from Sigma-Aldrich (St. Louis, Mo.). Reagents for cell culture were purchased from Gibco Invitrogen (Carlsbad, Calif.). Dialysis bags were obtained from Spectrum Laboratories, Inc. (Rancho Dominguez, CA). Water was deionized (dH₂O) on a Milli-Q water purification system (Millipore, Billerica, Mass.). Commercial MNP products used for in vitro stability comparisons, including fluidMAG-D (starch coating), fluid-MAG-CMX (carboxymethyl dextran coating), fluidMAGHeparin (heparin coating), and fluidMAG-DEAE (dextran diethylaminoethyl coating) were provided by Chemicell® (Berlin, Germany). These MNP products all possess a hydrodynamic diameter of approximately 100 nm.

### Preparation of Ga-MNP

Magnetite (Fe₃O₄) MNP were synthesized using a previously reported co-precipitation method (Kim et al., J Magn Magn Mater. 2001; 225:30-6). In brief, a solution containing ferric chloride and ferrous chloride was added dropwise to a 1.5 M NaOH solution under vigorous mechanical stirring and nitrogen gas protection at room temperature. The reaction temperature was gradually increased to 75° C. and held for 1 h under stirring and nitrogen gas protection. The synthesized MNP product were separated using a magnetic separator and washed five times with dH₂O. GA-MNP were prepared by adding 1 ml of the above MNP (10 mg Fe/ml) to 5 ml of 10% GA solution. The GA/MNP mixture was vortexed for 1 min and sonicated for 10 min to form a transparent colloidal solution. GA-MNP were then purified using a magnetic separator and washed five times with dH2O.

### Characterization of GA-MNP

Phase composition of lyophilized magnetic nanoparticles powder was analyzed with a Rigaku Rotoflex 200B 12 KW rotating anode X-ray diffractometer (RIGAKU, Inc., Tokyo, Japan) equipped with Cu-Kα radiation (λ=1.54056 Å). Morphology of GA-MNP was obtained on a JEOL 3010 high resolution transmission electron microscope (HR-TEM, JEOL, Ltd., Tokyo, Japan). Samples were prepared by dropping diluted particle suspensions on copper grids coated with formvar film (Ted Pella, Inc., Redding, Calif.) followed by drying at room temperature. Hydrodynamic particle size was measured by photon correlation spectroscopy using a PSS Nicomp 380 ZLS particle sizing system (Nicomp, Inc., Santa Barbara, Calif.). Iron concentration of all MNP samples was determined by inductively coupled plasma optical emission spectroscopy using an Optima 2000 DV spectrometer (Perkin-Elmer, Inc., Boston, Mass.). Magnetization measurements were performed using an MPMS-XL superconducting quantum interference device (SQUID) magnetometer (Quantum Design Inc., San Diego, Calif.). To measure relative GA content, GA-MNP were lyophilized and analyzed by thermogravimetric analysis (TGA) using a TGA-7 instrument (Perkin-Elmer, Inc.).

GA-MNP Stability and Comparison with Other Commercial MNP Products

In vitro stability of GA-MNP and other MNP products was assessed in a physiological simulated buffer of Dulbecco's phosphate-buffered saline (PBS, with 0.901 mM Ca₂⁺ and 0.493 mM Mg₂⁺) containing 10% fetal bovine serum (FBS). Briefly, MNP samples were added to the above solution and shaken to homogeneity (final iron concentration, 50 μg/ml). Immediately afterwards, aliquots containing 0.2 ml of the sample were loaded onto a 96-well plate, and the turbidity in the wells was measured at 400 nm using a micro-plate reader (Powerwave X340, BioTek Instruments, Inc., Winooski, Vt.) at 5-min intervals for 1 h (13). In addition, 3 ml of each test sample was added to a 20-ml glass scintillation vial for visual observation up through 24 h.

### Cell Viability Evaluation

Rat 9L glioma cells (Brain Tumor Research Center, University of California, San Francisco) were cultured at 37° C. under a humidified atmosphere of 5% CO₂ in Dulbecco's modified Eagle's medium (DMEM) supplemented with 10% FBS, 100 IU/ml penicillin, 100 μg/ml streptomycin, and 0.29 mg of L-glutamine. The cells were seeded on a 96-well plate at ˜10⁴ cells/well and incubated with fresh media for 24 h. Following incubation, the culture media were replaced with fresh media containing GA-MNP at various concentrations (0.01-20 mg/ml), and cells were further incubated at 37° C. for 4 h. The GA-MNP solution was then replaced with fresh media, and cell viability was measured after 24 h using a standard (3-(4,5-dimethylthiazol-2-yl)-2,5-diphenyltetrazolium bromide (MTT) assay (Fan et al., Nanotechnology. 2007; 18:195103).

### Cellular Uptake of GA-MNP

RhB-GA was prepared by conjugating rhodamine B (RhB) with GA through the formation of an amide bond as previously described (Fan et al., supra). Briefly, the GA solution was prepared by dissolving 2 g GA in 20 ml 0.1 M MES, whereas a RhB solution was prepared by dissolving 40 mg rhodamine B in 1 ml 0.1 M MES. The two solutions were then mixed, followed by the addition of 25 mg/ml EDC solution. The mixture was stirred for 2 h at room temperature. The RhBGA conjugates were then purified by dialysis using dH₂O for 3 days. The RhB-GA-MNP product were then prepared analogously to GA-MNP as described above.

To carry out cell uptake studies, rat 9L glioma cells were cultured in a 12-well plate and incubated with 80 μl of either GA-MNP or the RhB-GA-MNP (5 mg Fe per milliliter) in serum-free DMEM or DMEM with 10% FBS (120 μl) at 37° C. for 2 h. After incubation, the cells were washed five times with PBS and subjected to fluorescence and in vitro MR imaging.

To quantitatively compare the degree of cellular uptake between GA-MNP and a commercial starch-coated MNP (fluidMAG-D), rat 9L glioma cells were cultured in a 12-well plate and incubated with the two different MNP samples (at the same iron concentration ranging from 0.02 to 2.0 mg Fe per milliliter) in 1 ml DMEM containing 10% FBS at 37° C. for 2 h. Following incubation, the cells were washed five times with PBS, and cellular iron was measured by ESR spectroscopy according to a previously established protocol (Chertok et al., Biomaterials. 2008; 29:487-96). ESR spectra of samples were acquired using an EMX ESR spectrometer (Bruker Instruments Inc., Billerica, Mass.). Cells cultured without MNP were used as a control.

### In Vivo Study

All animal experiments were conducted according to protocols approved by the University of Michigan Committee on Use and Care of Animals.

9L glioma cells were grown to confluency, harvested, and resuspended in serum-free DMEM at a concentration of ˜104 cells per microliter. Ten microliters of cell suspension was implanted in the right forebrains of Fisher 344 rats (body weight, 200 g) at a depth of 3-4 mm beneath the skull through a 1-mm diameter burr hole. The surgical field was cleaned with 70% ethanol, and the burr hole was sealed with bone wax (Ethicon Inc., Summerfield, N.J.) to prevent extracerebral extension of the tumor (Huang et al., J. Mater Sci Mater Med. 2008; 19:607-14; Ross et al., Proc Natl Acad Sci USA. 1998; 95:7012-7).

MRI experiments were performed using a 12-cm horizontal bore, 7 Tesla Varian Unity Inova imaging system (Varian, PaloAlto, Calif.) according to a previously established procedure (Chertok et al., supra). Animals were anesthetized with 1.5% isoflurane/air mixture and imaged using a 35-mm diameter quadrature RF head coil (USA Instruments Inc., OH). MRI of the rat brain was initiated 10 days after tumor cell implantation. Axial sections of the rat brain were acquired with a T₂-weighted fast spin echo sequence using the following parameters: repetition time (TR)=4 s, echo time (TE)=60 ms, field of view=30×30 over 128×128 matrix, slice thickness=1 mm, slice separation=1.5 mm, four signal averages per phase encoding step. T₂-weighted images were inspected to determine which slice corresponded to the best cross-sectional visualization of the tumor. A single gradient echo (GE) scan was acquired at this optimized position to provide qualitative information on GA-MNP accumulation at the tumor site. GE images were acquired with the following parameters: TR=275.13 ms, TE=15 ms, field of view=30×30 over 128×128 matrix, slice thickness=1 mm.

For magnetic targeting, animals were anesthetized through inhalation of a 1.5% isoflurane/air mixture. Rats were then placed ventrally on a platform with the head positioned between the poles of an electromagnet. GA-MNP (in PBS) were injected at a dose of 12 mg Fe per kilogram through the tail vein and retained in the magnetic field for 30 min. Animals dosed with GA-MNP, but not subject to magnetic targeting, were used as controls. Animals were imaged with MRI before the administration of GA-MNP and after magnetic targeting (30 min after GA-MNP administration). To quantitatively examine the accumulation of GAMNP in the tumor and normal brain tissue, animals were killed immediately following targeting. Brain tumors were carefully dissected from the right hemisphere. Tumor and left hemisphere tissues were analyzed by ESR as previously described (Chertok et al., supra).

### Results

### GA-MNP Characterization

FIG. 35 showed the X-ray diffraction (XRD) pattern of GA-MNP with characteristic peaks of 2θ at 30.1°, 35.4°, 43.0°, 53.5°, 56.9°, and 62.6°, corresponding to the indices (220), (311), (400), (422), (511), and (440), respectively, of the magnetite crystal. In agreement with the results reported in the literature, XRD analysis confirmed that GA-MNP were composed of pure magnetite with spinal crystal structure (Hong et al., Nanotechnology. 2007; 18:13560).

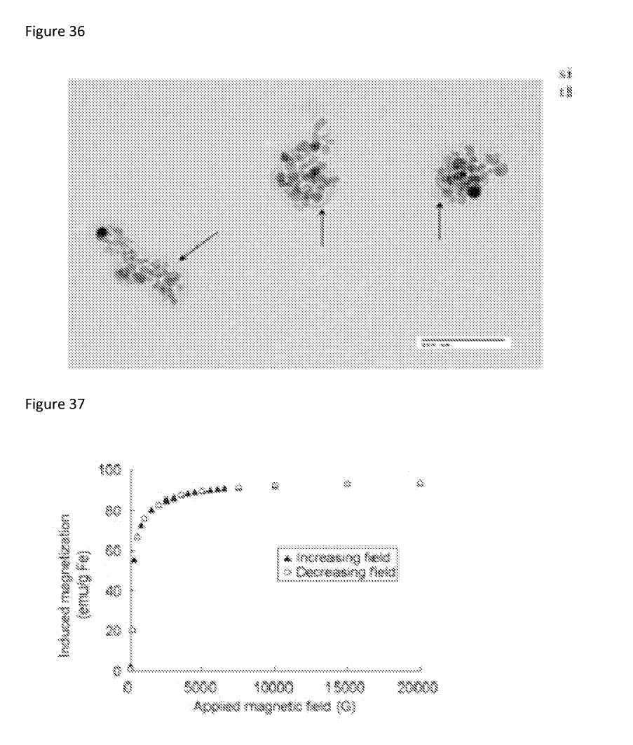

The morphology of GA-MNP was examined using transmission electron microscopy (TEM). TEM image in FIG. 36 visually showed the core-shell structure of GA-MNP. The cores consisted of a number of spherical magnetite nanocrystals with an average size of 14±3.8 nm coated with a shell of GA polymer. The multi-nanocrystal cores were formed because the GA molecules, much larger than the nanocrystals, could adsorb onto several nanocrystals through their carboxyl groups and hold them together. The relative GA content of GA-MNP by mass was found to be 15.6% by TGA.

For in vivo applications of magnetic nanoparticles, such as magnetic targeting and MRI, superparamagnetic behavior relevant. Superparamagnetic particles do not remain magnetized in the absence of an external magnetic field. Thus, they do not agglomerate without exposure to a field. MNP only exhibit superparamagentic behavior below a certain size threshold, namely the size of a single magnetic domain. The domain size for GA-MNP has been determined to be below the apparent limit of 25 nm (Sato et al., J Magn Magn Mater. 1987; 65:252-6). Negligible hysteresis was observed in magnetization experiments (FIG. 37), confirming that GA-MNP possessed superparamagnetic behavior. Overall, the synthesized GAMNP showed negligible coercivity (Hc) and remnant magnetization (Mr), as well as a saturation magnetization value of 93.1 emu/g Fe. These findings were all comparable to those of the commercial products obtained from Chemicell® (94 emu/g Fe) (Chertok et al., J Control Release. 2007; 122:315-23). Saturation magnetization was slightly lower than that of the bulk magnetite (127 emu/g Fe), probably due to the fact that the surface nonmagnetic layer (also known as “dead layer”) of GAMNP accounted for a higher composition fraction when the magnetite crystal size was reduced to nanosize (Willard et al., Int Mater Rev. 2004; 49:125-70). The saturation magnetization of GA-MNP was greater than that of the FDA-approved contrast agent Feridex® (70 emu/g Fe). It has been demonstrated that larger magnetite crystals yield stronger saturation magnetization (Jun et al., J Am Chem. Soc. 2005; 127:5732-3).

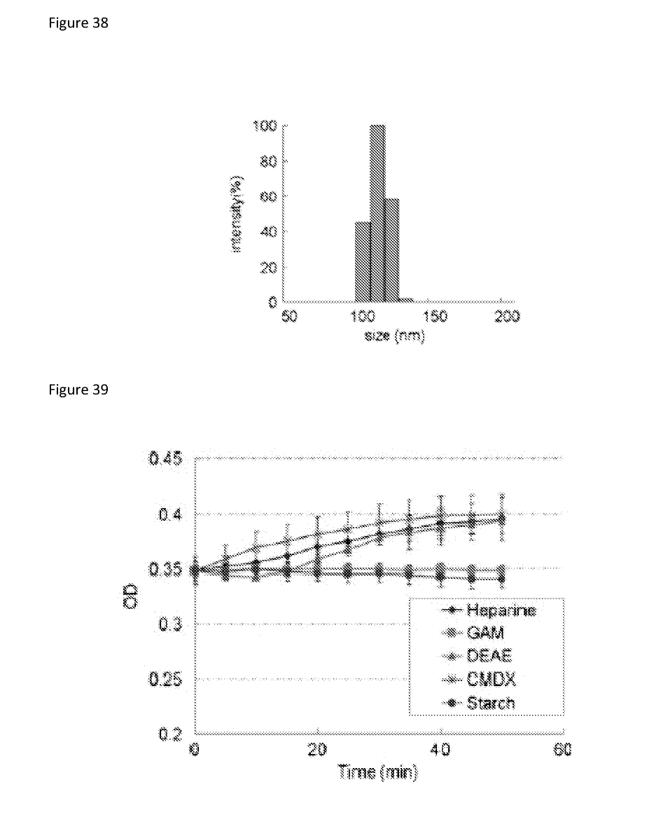

Particle size measurements (FIG. 38) showed that the synthesized GA-MNP possessed a mean hydrodynamic diameter of 118±12 nm in PBS buffer. It has been reported that attractive magnetic forces on MNP smaller than 50 nm are not sufficient to overcome forces from Brownian motion, resulting in poor MNP accumulation at the target site when subject to magnetic targeting (Yavuz et al., Science. 2006; 314:964-7). Thus, from a targeting standpoint, MNP with large core structures and hydrodynamic sizes are preferred. MNP over a certain size (e.g., >300 nm) are rapidly cleared from the body via the RES, resulting in a significantly shortened circulation half-life (Corot et al., Adv Drug Deliver Rev. 2006; 58:1471-504). The size cutoff of tumor vasculature permeation is on the order of several hundred nanometers (Arruebo et al., Nano Today. 2007; 2:22-32). Larger MNP and aggregates may not be able to extravasate into the interstitial space of the tumor, resulting in minimal tumor accumulation. It has been demonstrated that starch-coated MNP with a mean size of 100 nm were capable of extravasation and accumulation in tumor with the aid of magnetic targeting (Chertok et al., supra).

### Stability Evaluation and Comparison

MNP are typically administrated intravenously. Turbidity was used to monitor the stability of the suspensions (Petri-Fink et al., Eur J Pharm Biopharm. 2008; 68:129-3). All MNP samples investigated, including the GA-MNP product, remained stable in water for a period of several months. When mixed with physiologically simulated buffer, however, fluidMAGCMX, fluidMAG-Heparin, and fluidMAG-DEAE samples became turbid immediately after suspension, indicating rapid agglomeration (FIG. 39). The turbidity of GA-MNP and fluidMAG-D samples remained unchanged over the course of 1 h. GA-MNP suspension was clear even after 24 h of storage.

### Cellular Uptake of GA-MNP

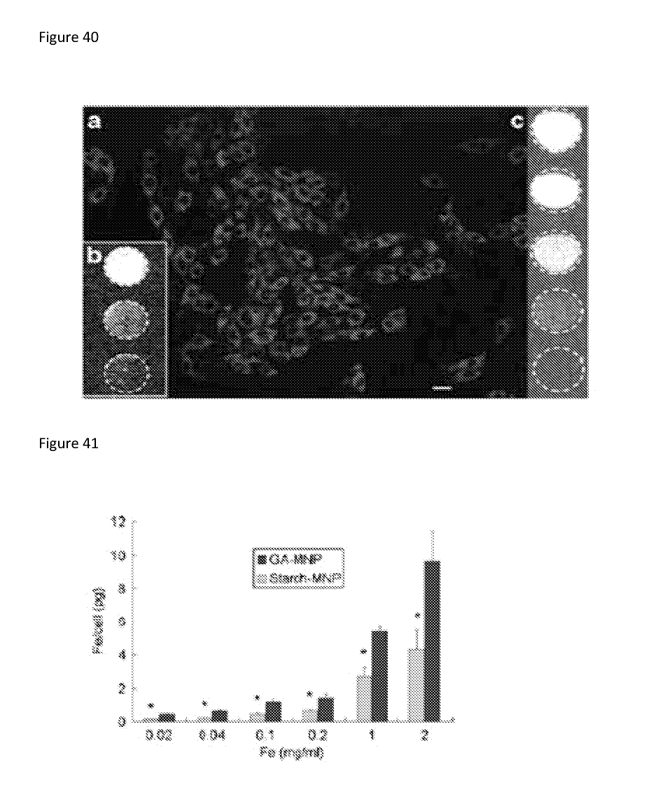

MNP and GA have been shown to be nontoxic and biocompatible (Kattumuri et al., Small. 2007; 3:333-41; Weissleder et al., Am J. Roentgenol. 1989; 152:167-73). The MTT assay results indicated that GA-MNP were not cytotoxic to 9L glioma cells in DMEM with and without FBS even at an iron concentration as high as 20 mg Fe per milliliter. Fluorescence microscopy demonstrated internalization of RhB-GA-MNP into 9L glioma cells 2 h after incubation (FIG. 40a). No fluorescence signal was found inside the nucleus, indicating that RhB-GA-MNP remains in the cytosol or endosome.

Consistent with results reported in the literature, in vitro GE MR images further confirmed cellular uptake of GAMNP (Lee et al., Nanotechnology. 2008; 19:165101.1-6). Pronounced hypointensity was observed in cells treated with GA-MNP when compared to the control, indicating the presence of GA-MNP inside the cells (see FIG. 40b). Furthermore, FIG. 40c revealed an iron concentration dependent drop in signal intensity on GE images for GAMNP, indicating higher cellular uptake of GA-MNP at higher concentrations.

Quantitative measurements of cellular uptake showed that both GA-MNP and starch-MNP exhibit a concentration dependent uptake of nanoparticles by 9L glioma cells (FIG. 41). The GA-MNP product displayed a two to threefold higher degree of uptake than starch-MNP when compared at the same iron concentrations. This result indicated that a GA-coated surface possesses a higher affinity toward tumor cells than that of starch.

### Brain Tumor Targeting and Imaging

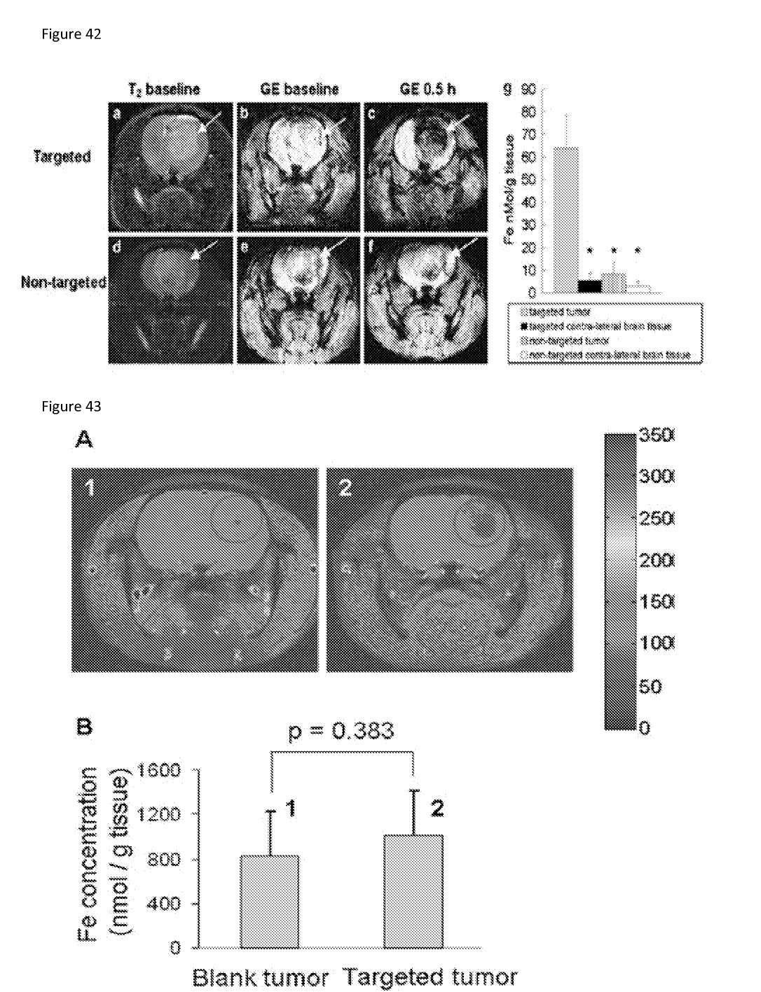

GE MR images qualitatively revealed accumulation GAMNP in the brains of animals subjected to magnetic targeting As shown in FIG. 42a, tumor was clearly visible on a T₂-weighted image. When compared with the GE baseline image shown in FIG. 42b, the pronounced hypointensity of the tumor observed in FIG. 42c demonstrated the accumulation of GA-MNP at the tumor site. No detectable hypointensity was found in other parts of the brain. Similar MRI studies were also conducted on brain tumor-harboring animals not subjected to magnetic targeting, as shown in FIG. 42d-f; no obvious signal change was observed in the GE image (FIG. 42f). The MR images qualitatively demonstrated that magnetic targeting improved brain tumor accumulation.

Quantitative tissue analysis by ESR further confirmed the selective accumulation of GA-MNP in tumor after magnetic targeting. For targeted animals (FIG. 42g), GA-MNP accumulation in the tumor tissue (63.8±14.6 nmol Fe per gram tissue, n=4) was 12-fold higher than that found in the contralateral brain tissue (5.3±3.5 nmol Fe per gram tissue, n=4). For animals not targeted, GA-MNP accumulation in tumors (8.4±5 nmol Fe per gram tissue, n=3) was threefold higher than that found in the contralateral brain (2.9±2.1 nmol Fe per gram tissue, n=3). Results from the control group indicated that the enhanced permeability of the tumor vasculature alone resulted in nanoparticle accumulation in tumors. With the application of the external magnetic field, though, tumor levels of MNP increased eightfold, indicating that magnetic targeting further enhanced nanoparticle accumulation.

### Example 7

### Comparison of Electron Spin Resonance Spectroscopy and Inductively-Coupled Plasma Optical Emission Spectroscopy for Biodistribution Analysis of Iron-Oxide Nanoparticles

### Materials and Methods

### Iron Oxide Nanoparticles

Iron oxide nanoparticles coated with starch (fluidMAG-D) were from Chemicell (Berlin, Germany). The physical properties of fluidMAG-D were previously analyzed (Chertok et al., supra).

### Animal Preparation

Cell Culture. Rat 9L-glioma cells were cultured and prepared for tumor induction as previously described (Chertok et al., supra). Briefly, 9L-glioma cells (Brain Tumor Research Center, University of California, San Francisco) were cultured in Dulbecco's modified Eagle's medium (DMEM) supplemented with 10% heat-inactivated fetal bovine serum, 100 IU/mL penicillin, 100 μg/mL streptomycin, and 0.29 mg of L-glutamine at 37° C. in a humidified atmosphere of 5% CO₂. Prior to implantation, cells were grown to confluency in 100 mm culture dishes and harvested using 0.25% trypsin/0.1% ethylene-diaminetetraacetic acid (EDTA) solution. Cells were pelleted by centrifugation at 1000 g for 5 min, resuspended in serum free DMEM at a concentration of ˜10⁵ cells/μL, and kept on ice until use.

Induction of Brain Tumors in Rat Model. Intracerebral 9L tumor induction was carried out as previously reported (Ross et al., Natl. Acad. Sci. U.S.A. 1998, 95 (12), 7012-7). Male Fisher 344 rats (125-150 g, Harlan Sprague-Dawley Inc., Indianapolis, Ind.) were anesthetized by intraperitoneal injection of ketamine/xylazine mixture (87/13 mg/kg body weight). A small skin incision was made over the right hemisphere, and the tissue was carefully removed until the bregma was identified. A 1-mm-diameter burr hole was drilled through the skull 1 mm anterior to the bregma and approximately 5 mm lateral from the midline. Ten microliters of 9L cell suspension was injected through the burr hole at a depth of 3 mm beneath the skull. The surgical field was cleaned with 70% ethanol, and the burr hole was filled with bone wax (Ethicon Inc., Summerfield, N.J.) to prevent extracerebral extension of the tumor, and the skin incision was closed with tissue adhesive (3M Vetbond, Animal Care products, St. Paul, Minn.). Animals were imaged using MRI (described below) beginning at 10 days postcell implantation to select tumors between 50 and 70 μL in volume as previously described (Ross et al., supra).

### Magnetic Targeting of Starch Coated Iron-Oxide Nanoparticles and Tissue Excision.

Starch-coated, iron oxide (magnetite) nanoparticles (fluidMAG-D) were magnetically targeted to brain tumors as described (Chertok et al., supra). Briefly, nanoparticle suspensions were diluted with PBS and filtered through a 0.2 μm disposable syringe filter to obtain a preparation of about 6 mg Fe/mL, as determined by ICP-OES described below. Tumor-bearing animals were anesthetized with an inhaled mixture of 1.5% isoflurane/air and tail veins cannulated using a 26-gauge angiocatheter (Angiocath™, Becton Dickinson, Sandy, Utah). The animals were then placed ventrally on a platform with their head positioned between the poles of an electromagnet. The magnetic field density within the air gap between the poles was adjusted to 0.4 T. Animals were then injected with nanoparticle suspension (12-25 mg Fe/kg) through the catheter placed in the tail vein and retained in the magnetic field for 30 min. Fifty minutes after nanoparticle administration, animals were sacrificed, and organs (brain, liver, spleen, lung, and kidney) were immediately excised for ex ViVo analysis. Tumors were carefully dissected from the right brain hemisphere. Animals not exposed to nanoparticles or magnetic targeting served as controls. All tissues were stored at −80° C. prior to analysis.

### Magnetic Resonance Imaging (MRI)

MRI experiments were performed on an 18-cm horizontal-bore, 7 T Varian Unity Inova imaging system (Varian, Palo Alto, Calif.). Animals were anesthetized with 1.5% isoflurane/air mixture and imaged using a 35-mm-diameter quadrature RF head coil (USA Instruments Inc., OH). To visualize the tumor localization within the rat brain, 13 axial sections of the brain were acquired with a T₂-weighted fast spin echo sequence using the following parameters: repetition time (TR)=4 s, echo time (TE)=60 ms, field of view=30×30 over 128×128 matrix, slice thickness=1 mm, slice separation=2 mm, and four signal averages per phase encoding step. To determine nanoparticle distribution in the brain, 13 gradient echo (GE) axial slices of the brain were collected before the nanoparticle administration (baseline scans) and immediately following magnetic targeting. GE images were acquired with the following parameters: TR=20 ms, TE=5 ms, field of view=30×30 over 128×128 matrix, and slice thickness=1 mm.

### Ex Vivo ESR Analysis of Tissues

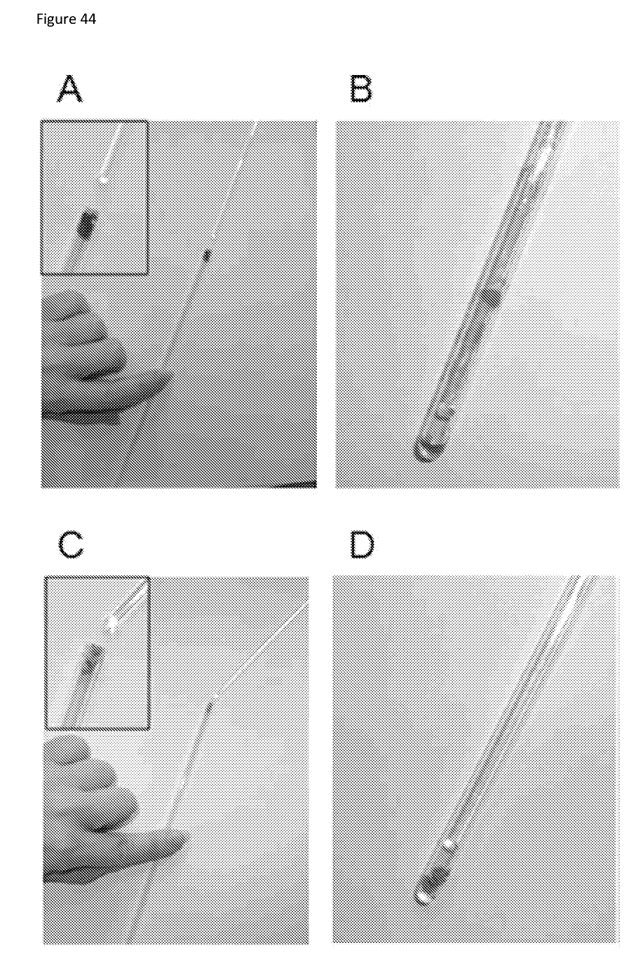

Excised tissue samples (n=7-8) were first subjected to ESR spectroscopy to determine MNP content. Samples from animals (n=5) not exposed to nanoparticles were also analyzed to assess background. Frozen tissue samples (−30 mg) were sectioned into 2 mm×2 mm cubes using a razor blade. Sections were applied to the top of an ESR tube, precooled on dry ice, and quickly pushed to the tube bottom using a thin glass rod (see FIG. 44). Tubes were weighed before and after packing to determine tissue sample mass. For each excised organ, triplicate tissue samples were packed for analysis. Magnetic susceptibility of MNP and consequently the magnitude of the ESR signal (per unit iron), varies across different MNP preparations due to differences in structural attributes (e.g., core morphology, size distribution, and composition of surface coating).The ESR signal was calibrated with the investigated MNP species. Therefore, calibration standards were prepared, in triplicate, from the same MNP stock used for in ViVo studies. Iron concentration of the stock was determined by ICP-OES. Stock dilutions (100 μL) were carefully applied to the bottom of ESR tubes (to prevent sample loss on tube walls) with a microsyringe coupled to a 12 in Teflon needle (Wilmad Labglass, Buena, N.J.). Tissues samples and calibration standards were analyzed on an EMX ESR spectrometer (Bruker Instruments, Billerica, Mass.) equipped with a liquid nitrogen cryostat. Spectra were acquired using the following parameters: resonant frequency=˜9.3 GHz; microwave power=20 mW; and temperature=−128° C. (Mykhaylyk et al., J. Magn. Magn. Mater. 2001, 225 (1-2), 241-247). Receiver gain and modulation amplitude varied depending on the tissue. Spectra were obtained as the first derivative (dP/dB) of absorbed microwave power (P) with respect to applied magnetic field (B). The double integral of the first derivative signal (∫∫((dP)/(dB))dBdB) is known to be proportional to the number of resonating electronic spins in a measured sample. Double integrals of acquired spectra (DI) were calculated using the MathCAD software package (PTC Corp, Needham, Mass.). DI values of MNP-containing tissues were normalized by sample weight and background corrected by subtracting weight-normalized DI of corresponding blank tissues (obtained from animals not exposed to MNP). Tissue MNP concentrations (nmol Fe/g tissue) were recalculated from weight-normalized background-corrected DI values using calibration curves constructed with MNP standards.

### Ex Vivo ICP-OES Analysis of Tissues

Tissue samples, initially analyzed by ESR, were then studied by ICP-OES, to minimize any variability that may arise from analyzing different tissue cuts. ESR tubes were scored with a diamond scribe and cracked open just above the tissue crown. Tube pieces containing tissue were placed on dry ice for 1 min to refreeze thawed tissue. While still frozen, tissues were carefully extracted using forceps and weighed in 2 mL vials containing 1.0 mm high-density Zirconia beads (500 μL, Biospec Products, Bartlesville, Okla.). Tween 80 (0.1% in Milli-Q water, 500 μL) was added, and the mixture was homogenized with a Mini-BeadBeater-8 homogenizer (Biospec Products, Bartlesville, Okla.). Concentrated hydrochloric acid (12M, 2 mL) was added to the homogenate (350 μL), and the mixture was digested for 2 h at 70° C. Milli-Q water and yttrium internal standard (GFS Chemicals, Columbus, Ohio) were then added for a total volume of 4 mL containing 1 mg/L yttrium. Digestion mixtures were then vortexed and filtered through a 0.45 μm syringe filter (Titan2 reversed cellulose, SunSri, Rockwood, Tenn.) to remove solid content. Analysis of a high MNP content liver homogenate revealed no statistically significant difference in measured iron between filtered (11.9±1.9 nmol Fe/mg tissue) and nonfiltered (11.6 nmol±1.8 Fe/mg tissue) preparations. Iron content was determined from emission readings at Fe 238.204 nm emission line using an ICP-OES spectrometer (Optima DV 2000, PerkinElmer, Waltham, Mass.). The instrument was calibrated with dilutions of an iron standard (GFS Chemicals, Columbus, Ohio) also spiked with yttrium internal standard (1 mg/L). Raw ICP results were calculated with WinLab32 for ICP software (Perkin-Elmer, Waltham, Mass.) and used to backcalculate the amount of iron in the original homogenate. The mass of digested tissue was calculated based on the ratio of digested (350 μL) to total (500 μL) homogenate volume and the original mass of tissue. As with the ESR analysis, tissue iron amounts were normalized by weight and averaged to obtain a total iron concentration (nmol Fe/g tissue) for each organ. Data were then corrected for endogenous iron by subtracting a normalized average iron concentration determined for blank tissues (n=5) not exposed to MNP.

### Statistical Analyses

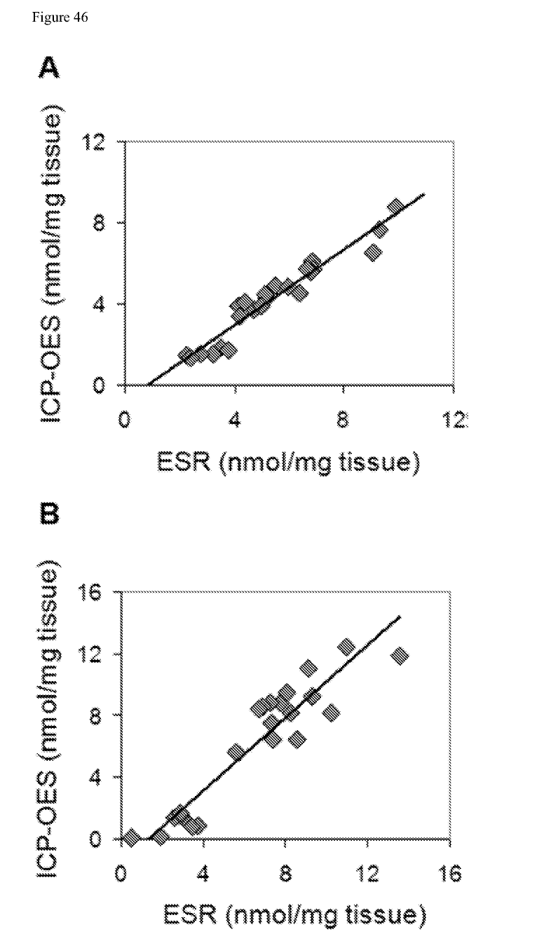

All data are presented as mean±SD unless otherwise noted. Statistical comparison of ICPOES measured iron content in blank and targeted tumors (FIG. 43B) was made using the Student's t test with a significance of p<0.05. Correlations of liver and spleen data from ESR and ICP analyses (FIGS. 46A,B) were determined as Pearson correlation coefficients (r) using SPSS17.0 Software (SPSS, Chicago, Ill.).

### Results

### Initial ICP-OES Analysis of Excised Brain Tumors

ICP-OES is widely utilized to study the biodistribution of iron-oxide nanoparticles. Animals were imaged with MRI after magnetic targeting to visually confirm the presence of MNP in tumors prior to tissue excision. FIG. 43A shows representative gradient echo (GE) MRI head scans of animals not exposed to MNP (panel 1) and those magnetically targeted (panel 2). Tumor lesions were identified with T₂ weighted MRI and are highlighted with circles. Magnetic nanoparticles are strong enhancers of T₂/T₂* proton relaxation and thus manifest themselves by pronounced hypointensity (negative contrast) on GE MRI scans. As seen in FIG. 43, in contrast to blank tumors which show very little signal reduction, pronounced hypointensity can be observed in targeted tumors indicating nanoparticle accumulation.

Both blank and targeted tumors showed similar levels of iron according to quantitative ICP-OES analysis (FIG. 43B). No statistically significant difference (p=0.38) in iron content could be detected between the two groups. The level of iron measured in blank tumors was more than 25 times greater than the amount attributed to targeted MNP (Chertok et al., supra).

### Cryogenic Methodology for Tissue Packing of ESR Tubes

ESR is a useful methodology for assaying iron-oxide in tissues due to its sensitivity for paramagnetic species. Before comparing the ESR and ICP-OES tissue needs to be introduced into the bottom of a long, narrow ESR tube. Small tissue cuts were first loaded at ambient temperature into the tubes. Cuts were easily applied to the tops of ESR tubes as shown in FIG. 44A. Upon pushing cuts further into the tube (FIG. 44B); however, tissue smeared along the tube walls. Only a small fraction of the tissue shown in FIG. 44A could be delivered to the bottom of the tube. To overcome this problem, a cryogenic method for tissue handling was developed. Exhibited in FIG. 44C, frozen tissue cuts were loaded into the tops of ESR tubes. When quickly pushed with the glass rod, nearly all tissue shown in FIG. 44C could be delivered to the bottom of the tube (FIG. 44D). No visible smearing was observed. The methodology was also, nondestructive rendering samples available for additional analyses (e.g., ICP-OES) upon extraction from tubes.

### ESR Analysis of Tissues for MNP Content

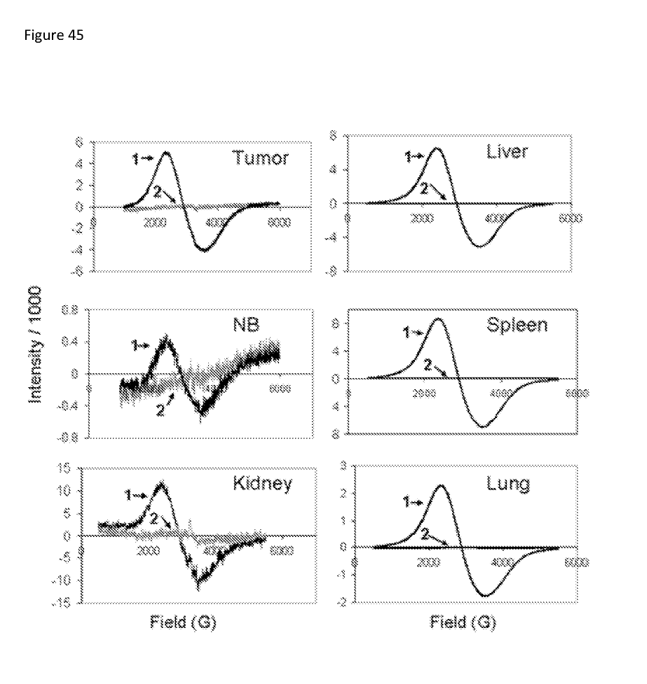

ESR analysis of various tissues was conducted using the cryogenic sample handling procedure described above. Liver, spleen, lung, brain, tumor, and kidney were chosen for analysis. Representative spectra for each organ are shown in FIG. 45. The spectra of organs from MNP injected animals exhibit a broad resonance signal centered at about g=2.2 with a line width of about 1100 Gauss. The shape and field location of this signal are identical to those of the standard MNP suspensions (Chertok et al., supra) and are consistent with ESR spectra of superparamagnetic iron oxide nanoparticles reported in the literature (Koseoglu et al., Physica Status Solidi C 2004, 1 (12), 3516-3520). The signal intensity contributions from all background sources is negligible compared to that of MNP containing tissues. The only visible signal, seen in the kidney control, is a low-intensity, narrow peak (g=2.0, line widthₚ₋ₚ=240 Gauss), which can be attributed to ferritin (Slawska-Waniewska et al., J. Magn. Magn. Mater. 2004, 272-276 (Part 3), 2417-2419). The test spectra were distinct from blanks not only for relatively high but also for relatively low accumulating tissues, revealing high ESR sensitivity to MNP in tissue samples.

### Comparison of ESR and ICP-OES Methodologies

The ability of ESR and ICP-OES methods to quantify nanoparticle accumulation was next compared for two groups of organs with high or low MNP accumulation. Animals were dosed with varied amounts of MNP to produce a range of tissue nanoparticle concentrations in each of the different organs tested. In addition, single organ ICP-OES and ESR analyses were performed on the same tissue sample to minimize the effect of accumulation heterogeneity across an organ. Liver and spleen samples were first compared. As seen in FIG. 46A, the ICP-OES and ESR data obtained from liver samples followed a strong, positive linear correlation (r=0.97, p<0.01) with a negative intercept. Data obtained from spleen tissue (FIG. 46B) were similarly correlated (r=0.94, p<0.01), also with negative intercept. The high correlations shown in FIG. 46 indicated that the two methods were equivalent in evaluating tissues that accumulated high levels of MNP. The negative intercepts shown in FIG. 46 indicated that ICP-OES was insensitive to MNP when tissue accumulation became relatively low.

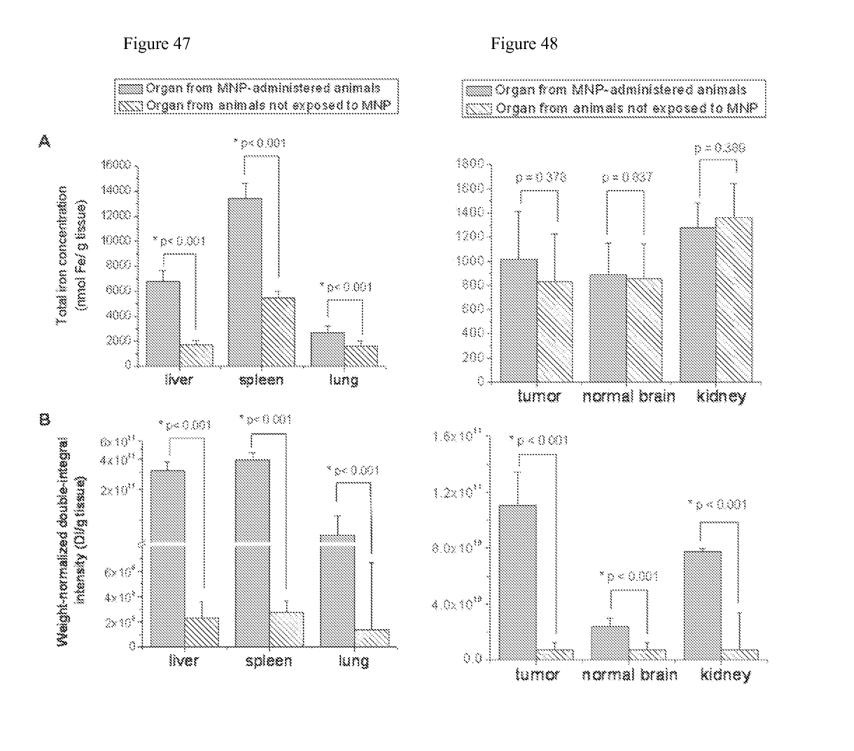

The differences between the experimental (with administration of MNP) and background (without administration of MNP) signal for both ESR and ICP-OES were compared. FIG. 47 demonstrates that in high-MNP accumulating tissues the signal generated by MNP-exposed organs was significantly higher (p<0.001) than the background from the corresponding blank organs (not exposed to MNP) with both the ICPOES (FIG. 47A) and the ESR (FIG. 47B) methodologies. Thus, the experimental tissue signal could be corrected for background to calculate the fraction of the signal corresponding to exogenous MNP. In the case of high-accumulating tissues, this procedure can be reliably performed with either methodology, explaining the strong correlation between ESR and ICP-OES in MNP determination for liver and spleen (FIG. 46).

The analysis outcomes with ESR and ICP-OES for low accumulating tissues did not show the same agreement (FIG. 48). Total iron concentrations determined for MNP-exposed tumor, normal brain and kidney tissues with ICP-OES did not significantly differ (p>0.05) from the background total iron in the corresponding blank organs (FIG. 48A). High levels of background iron masked exogenous content, thus compromising MNP detection. ESR analysis of low accumulating tissues revealed a different pattern. Weight-normalized double integrated intensities of the ESR signal from MNP-exposed tissues were found to be significantly higher (p<0.001) than the corresponding background values (FIG. 48B). The discrepancy in ESR and ICP-OES analyses of low accumulating tissues demonstrates that contribution of endogenous iron species to the ESR signal does not scale with their elemental iron content. Thus, ESR successfully resolved MNP accumulation in the brain, tumor, and kidney tissues of targeted animals.

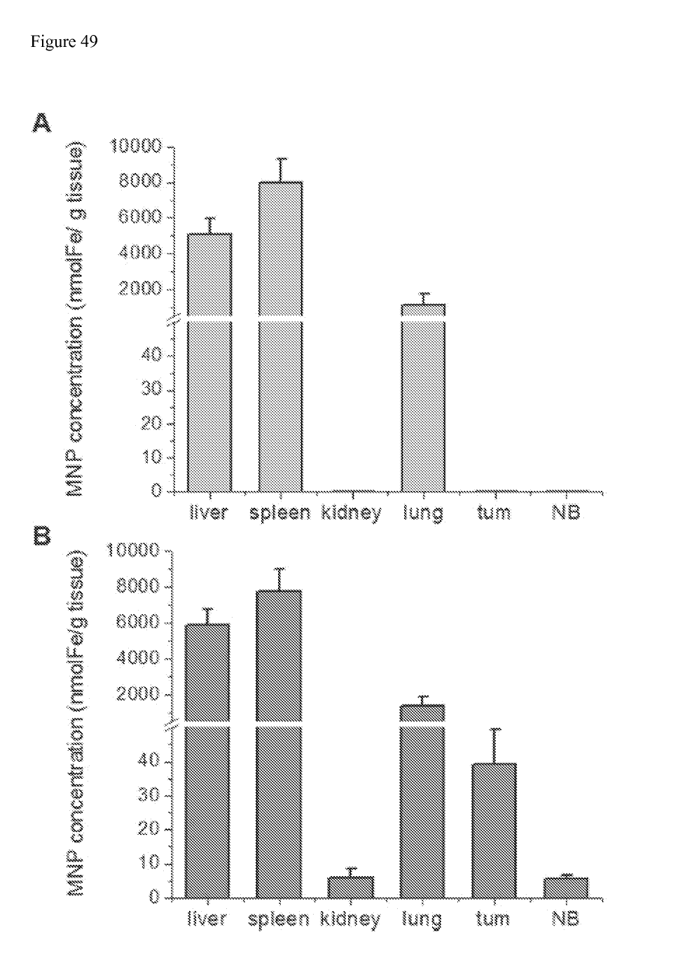

The differences between the ESR and ICP-OES methods on biodistribution analysis is shown in FIG. 49. MNP were successfully detected in high accumulating organs (liver, spleen, and lung) by both methodologies; ICP-OES was insensitive to MNP in the tumor, brain, and kidney (FIG. 49A), yielding an incomplete profile of biodistribution. When the analysis was performed with ESR (FIG. 49B, the MNP content of the tumor (39.2±10.3 nmol Fe/g tissue) and also of the kidney (6.1±2.4 nmol Fe/g tissue) and normal brain (5.6±0.8 nmol Fe/g tissue) could be successfully determined with ESR.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, genetics, or related fields are intended to be within the scope of the following claims.

What is claimed is:

1. A system for targeting brain tumors, comprising
a) magnetic iron oxide nanoparticles (MIONs) coated with a coating molecule, wherein said coating molecule is non-covalently associated with a brain targeting molecule comprising anti-tumor agent linked to a cell-penetrating peptide; and
b) an external magnetic field configured to orient said MIONs at the site of said brain tumor.

2. The system of claim 1, wherein said cell-penetrating peptide comprises a protein transduction domain peptide.

3. The system of claim 2, wherein said protein transduction domain peptide is selected from the group consisting of TAT, low molecular weight protamine, and arginine-rich peptides.

4. The system of claim 1, wherein said brain targeting molecule comprises polyethyleneime polymer.

5. The system of claim 1, wherein said protein transduction domain is low molecular weight protamine.

6. The system of claim 1, wherein said system further comprises an agent that disrupts the association between said MION and said brain targeting molecule.

7. The system of claim 6, wherein said agent is protamine.

8. The system of claim 1, wherein said coating molecule is a sulfated glycosaminoglycan.

9. The system of claim 8, wherein said sulfated glycosaminoglycan is selected from the group consisting of heparin, heparin sulfate, dextran sulfate and a chondroitin sulfated hyaluronic acids.

10. The system of claim 9, wherein said sulfated glycosaminoglycan is heparin.

11. The system of claim 1, wherein said system further comprises a permanent magnet mounted to a tapered pole of a dipole electromagnet.

12. The system of claim 11, wherein said permanent magnet diverts the magnetic flux lines emanating from the electromagnet poles to generate a local maximum of the magnetic field on the exposed pole magnetic flux density.

13. A method of targeting brain tumors, comprising:
a) administering magnetic iron oxide nanoparticles (MIONs) coated with a coating molecule, wherein said coating molecule is non-covalently associated with an brain targeting molecule comprising anti-tumor agent linked to a cell-penetrating peptide to a subject diagnosed with a brain tumor; and
b) orienting said MIONs at the site of said tumor with an external magnetic field.

14. The method of claim 13, wherein said coating molecule is a sulfated glycosaminoglycan.

15. The method of claim 14, wherein said sulfated glycosaminoglycan is selected from the group consisting of heparin, heparin sulfate, dextran sulfate and a chondroitin sulfated hyaluronic acids.

16. The method of claim 15, wherein said sulfated glycosaminoglycan is heparin.

17. The method of claim 13, further comprising the step of administering an agent that disrupts the association between said Mion and said brain targeting molecule.

18. The method of claim 13, wherein said administering is intra-arterial administration.

19. The method of claim 18, wherein said intra-arterial administration comprises inserting a capillary tube into the artery under conditions such that blood flow through said artery is not impeded.

20. The method of claim 18, further comprising the step of utilizing a permanent magnet mounted to a tapered pole of a dipole electromagnet to divert the magnetic flux lines emanating from the electromagnet poles to generate a local maximum of the magnetic field on the exposed pole magnetic flux density.

21. The method of claim 20, wherein said method prevents the formation of vascular embolisms at the site of said intra-arterial administration.
